(19) United States
(12) Patent Application Publication
(10) Pub. No.: US 2019/0381165 A1
(43) Pub. Date: Dec. 19, 2019

(54) Vaccines and Diagnostics for Novel Porcine Orthoreoviruses

(71) Applicants: Xiang-Jin Meng, Blacksburg, VA (US); Dianjun Cao, Blacksburg, VA (US); Athmaran Narayanappa, Blacksburg, VA (US)

(72) Inventors: Xiang-Jin Meng, Blacksburg, VA (US); Dianjun Cao, Blacksburg, VA (US); Athmaran Narayanappa, Blacksburg, VA (US); Elankumaran Subbiah, Blacksburg, VA (US)

(73) Assignee: Virginai Tech Intellectual Properties, Inc., Blacksburg, VA (US)

(21) Appl. No.: 16/557,210

(22) Filed: Aug. 30, 2019

Related U.S. Application Data
(63) Continuation-in-part of application No. 15/527,670, filed on May 17, 2017, now abandoned.
(60) Provisional application No. 62/080,462, filed on Nov. 17, 2014.

(51) Int. Cl. A61K 39/15; C07K 14/005; C12N 7/00

(52) U.S. Cl. CPC A61K 39/15; A61K 2039/5252; C12N 7/00; C07K 14/005

(57) ABSTRACT

Provided herein are diagnostics and vaccines to identify control and prevent novel porcine orthoreovirus type 3 (POV3) isolated from diarrheic feces of piglets from outbreaks in three states and ring-dried swine blood meal from multiple sources.

### CROSS REFERENCE TO RELATED APPLICATIONS

This application claims priority as a continuation-in-part application to U.S. application Ser. No. 15/527,670, filed May 17, 2017, which was a 35 U.S.C. § 371 application based on PCT/US2015/061034, filed Nov. 17, 2015, which in turn claims priority based on U.S. Provisional Application Ser. No. 62/080,462 filed Nov. 17, 2014, each of which are incorporated by reference in their entireties.

### REFERENCE TO SEQUENCE LISTING SUBMITTED ELECTRONICALLY

The Sequence Listing associated with the application is provided in text format in lieu of a paper copy, and is hereby incorporated by reference into the specification. The name of the text file containing the Sequence Listing is SequenceListing.txt. The text file (ASCII) is 209 kilobytes, was created on Aug. 30, 2019 and is being submitted electronically via EFS-Web.

### FIELD OF THE INVENTION

This invention relates generally to compositions and methods for diagnosis and prophylactic vaccines for newly emerging mammalian orthoreoviruses that cause considerable mortality and morbidity in swine farms.

### BACKGROUND OF THE INVENTION

Without limiting the scope of the invention, its background is described in connection with recent outbreaks of epidemic diarrhea in swine populations. In May 2013, a devastating outbreak of epidemic diarrhea in young piglets commenced in swine farms of the United States, causing immense economic concerns. The mortality can reach up to 100% in piglets less than 10 days of age, with a recorded loss of at least 8 million neonatal pigs since 2013. Enteric coronaviruses, such as swine enteric coronaviruses (SECoVs), porcine epidemic diarrhea virus (PEDV), and porcine deltacoronavirus (PDCoV), were isolated from these outbreaks and characterized. However, despite intensive biosecurity measures adopted to prevent the spread of SECoV in many farms and the use of two U.S. Department of Agriculture (USDA) conditionally licensed vaccines against PEDV, the outbreaks have continued and have now spread to many other countries, including Mexico, Peru, Dominican Republic, Canada, Columbia, and Ecuador in the Americas and Ukraine. Repeated outbreaks have also been reported on the same farms that were previously infected with PEDV. In June 2014, the USDA issued a federal order to report, monitor, and control swine enteric coronavirus disease (SECD).

Porcine orthoreoviruses are also known to cause diarrhea in swine populations and outbreaks have been reported in China and Korea but not in the United States. The family Reoviridae comprises 15 genera of double-stranded RNA (dsRNA) viruses. Orthoreoviruses are a genus within the Reovirus family in the subfamily Spinareovirinae. There are five species within the Orthoreovirus genus with Mammalian ortheoreovirus (MRV) being the type species. There are three serotypes of MRV: MRV1, MRV2, and MRV3. This virus species is characterized by a segmented double stranded RNA genome within a non-enveloped, icosahedral virion with a double capsid structure.

The segmented MRV genome has 10 discrete RNA segments which is divided into three size classes: three large segments (L1, L2 and L3), three medium segments (M1, M2 and M3), and four small segments (51, S2, S3 and S4), encoding three λ, three μ, and four σ proteins, respectively. MRV have been isolated from a wide variety of animal species, including bats, civet cats, birds, reptiles, pigs, and humans. Most orthoreoviruses are recognized to cause respiratory infections, gastroenteritis, hepatitis, myocarditis, and central nervous system disease in humans, animals, and birds. Orthoreovirus genomes are prone to genetic reassortment and intragenic rearrangement. The exchange of RNA segments between viruses can lead to molecular diversity and evolution of viruses with increased virulence and host range. MRV serotypes 1 to 3 were associated with enteritis, pneumonia, or encephalitis in swine around the world, including China and South Korea. The zoonotic potential of MRV3 has been reported recently. However, porcine orthoreovirus infection of pigs was unknown previously in the United States.

From the foregoing, it appeared to the present inventors that a new infectious agent might be involved in the outbreaks. Provided herein is the discovery of novel infectious agents causing epidemic diarrhea in swine as well as assays for detection and preventive vaccines.

### BRIEF SUMMARY OF THE INVENTION

The present invention is directed to assays for diagnosis and prevention of a novel porcine orthoreovirus type 3 (POV3-VT) that the present inventors determined to be a causative agent in diarrheic piglet outbreaks in three states. The agent was identified in ring-dried swine blood meal from multiple sources. In order to combat this new agent, the present inventors have developed methods for detection of the virus in multiple samples, antibodies to the virus in pig populations and vaccines for prevention of the disease.

In certain embodiments a vaccine that confers immunity to POV3-VT is provided that includes an immunogenic amount of one or more type specific POV3-VT proteins or immunogenic portions thereof. In certain embodiment the type specific POV3-VT proteins are selected from the group consisting of σ1, σ1s, μ1 and μ2 proteins and immunogenic portions thereof. The immunogenic proteins may be presented in a number of different ways including via a live attenuated virus vaccine, a killed virus vaccine and a subunit vaccine. Preferable subunit vaccines are generated by in vitro production of the immunogenic proteins in bacterial or baculovirus cells.

Also provided are vaccines that confers immunity to POV3-VT including an immunogenic MRV3 σ1 protein, or an immunogenic polypeptide portion thereof, wherein the MRV3 σ1 protein has at least 92% identity with amino acid residues 1 to 455 of SEQ ID NO: 20.

Attenuated live virus vaccines are provided wherein the vaccine is developed by passage of a POV3-VT virus in a non-porcine host until the passaged virus is capable of conferring immunity when inoculated into pigs but incapable of causing epidemic diarrhea.

In certain embodiments, a method of detecting an infection of an animal by a POV3-VT virus is provided including providing a sample from the animal, and detecting the presence or absence in the sample of an antibody that specifically binds to a polypeptide comprising a POV3-VTt σ1 protein, or an immunogenic polypeptide portion thereof (SEQ ID NO: 20), wherein the detecting of the presence or absence in the sample of an antibody that specifically binds to the polypeptide comprises use of an antibody-based technique capable of detecting the specific binding of an antibody to a protein, and the detecting of the specific binding of an antibody in the sample to the polypeptide detects infection of the animal by the POV3-VT virus. The method may be an immunohistochemistry assay, a radioimmunoassay, an ELISA (enzyme linked immunosorbant assay), a sandwich immunoassay, an immuno-radiometric assay, a gel diffusion precipitation reaction, a immunodiffusion assay, an in situ immunoassay, a Western blot, a precipitation reaction, an agglutination assay, a complement fixation assays, a immunofluorescence assay, a protein A assay, and an immunoelectrophoresis assay.

In other embodiments a process of detecting POV3-VT in a biological sample is provided including producing an amplification product by amplifying a POV3-VT S1 segment nucleotide sequence using forward and reverse primers homologous to regions within the S1 segment of POV3-VT under conditions suitable for a polymerase chain reaction and measuring said amplification product to detect POV3-VT in said biological sample. In other embodiments the method of detection of POV3-VT in a biological sample includes producing an amplification product by amplifying a plurality of targets including a POV3-VT S1 segment and at least one additional POV3-VT segment selected from the group consisting of S2, S3, S4, L1, L2, L3, M1, M2 and M3 segments, each amplification using forward and reverse primers homologous to regions within each respective segment of POV3-VT under conditions suitable for a polymerase chain reaction; and detecting the amplification products to detect POV3-VT in said biological sample.

In certain embodiments, the POV3-VT is detected in feed supplements and by detecting the presence of the virus in feed supplements, contamination with live virus can be avoid either by refusing use of the contaminated supplements or by further testing the supplements to determine whether live virus is present. Combinations of sensitive testing for the presence of viral DNA/RNA coupled with further selective testing for live virus not only allows avoidance of contaminated feed but also allows the development of techniques able to fully inactivate potentially contaminated feed supplements.

Also provided herein is a probe for the detection of a POV3-VT virus nucleic acid that comprises a nucleotide sequence having at least 98% sequence homology with the unique S1 segment (SEQ ID NO: 19) of POV3-VT together with a label. The probe may thus be radiolabeled, fluorescently-labeled, biotin-labeled, enzymatically-labeled, or chemically-labeled. The POV3-VT virus nucleic acid may be amplified for detection by polymerase chain reaction (PCR), real-time PCR, reverse transcriptase-polymerase chain reaction (RT-PCR), real-time reverse transcriptase-polymerase chain reaction (rt RT-PCR), ligase chain reaction, or transcription-mediated amplification (TMA).

### BRIEF DESCRIPTION OF THE DRAWINGS

For a more complete understanding of the present invention, including features and advantages, reference is now made to the detailed description of the invention along with the accompanying figures:

FIG. 1A shows the RNA profile of the novel FS03 and BM100 U.S. porcine MRV3 (“POV3”) genome segments on a 7.5% SDS-PAGE gel. FIG. 1B depicts the protein profile of FS03 purified virus on 7.5% SDS-PAGE gel. FIG. 1C shows the temperature sensitivity of POV3 isolates F503 and BM100. The TCID₅₀ virus titers (mean values±standard deviation) after treatment at different temperatures (34, 37, 56, 80, and 90° C.) are plotted along with that of the untreated virus control (VC). Differences in the titers were evaluated by two-tailed t test, and statistically significant (P<0.05) titers of F503 ($) and BM100 (*) are indicated.

FIG. 2A shows that the POV3 disclosed herein induce syncytia in BHK-21 cells. FIG. 2A shows mock-infected BHK-21 cells, while FIG. 2B shows BHK-21 cells infected with T3/Swine/FS03/USA/2014 (FS03) virus showing syncytia (arrows) at 48 hpi. FIG. 2C shows transmission electron microscopy (TEM) analysis infected Vero cells wherein the presence of paracrystalline arrays of virus particles free of organelles and viral factories in the cytoplasm was evident. Negatively stained virions revealed icosahedral, nonenveloped, double-layered uniform sized particles reminiscent of members of the family Reoviridae. The mean diameter of the virus particles was 82 nm (FIG. 2C inset), with particle sizes ranging from 80 to 85 nm.

FIG. 3 provides an alignment of the S1 segment encoded σ1 protein amino acid sequences of F503 and BM100 POV3 in comparison to T3Dearing, T3/Bat/Germany, T1L (Lang), and T2J (Jones) isolates. The novel F503 and BM100 POV3 viruses possessed 31 and 11 unique amino acid substitutions in the σ1 and σ1s proteins in comparison to T3/Bat/Germany and other MRV prototypes. Deduced amino acid sequence analysis of σ1 protein revealed that the sialic acid binding domain (NLAIRLP), and protease resistance (249I) and neurotropism (340 D and 419E) residues were conserved in the U.S. porcine orthoreovirus (POV3) strains.

FIG. 4 provides an alignment of the M2 segment encoded μ1 protein amino acid sequences of FS03 and BM100 POV3 in comparison to T3Dearing,/Bat/Germany, T1L (Lang), and T2 (Jones). The sequence alignment of the μ1 protein indicated 6 amino acid substitutions that were unique to these isolates in comparison to the T3/Bat/Germany, T3D, T1L, and T2J isolates).

FIG. 5 provides an alignment of the M1 segment encoded μ2 protein amino acid sequences of FS03 and BM100 POV3 in comparison to T3Dearing, T3/Bat/Germany, T1L, and T2J. The μ2 protein alignment revealed 15 unique amino acid substitutions compared to the T3/Bat/Germany, T3D, T1L, and T2J sequences and possessed the S208P mutation compared to T3/Dearing.

FIG. 6A shows POV3 inactivation over time using 1 mM BEI. FIG. 6B shows POV3 inactivation over time using 2.5 mM BEI.

FIG. 7A shows the HI titers of 450 samples plotted in 2 Log scale.

FIG. 7B depicts ELISA results obtained for randomly selected 59 unknown pig sera samples from the 2014 outbreak in Ohio, 31 known negative pig sera samples from the year 2008 are represented in the figure.

FIG. 8 show PT_PCR results with POV3 specific primers. The amplified length was 424 bp and 537 bp for S1 and L1 gene fragments respectively. M: 1 Kb+ ladder, Lane 1-2: POV3—Fecal sample (S1 target), Lane 3: POV3—Blood meal (S1 target), Lane 4: No template negative control, Lane 5: POV3—Fecal sample (L1 target), Lane 6: POV3—Blood meal (L1 target).

FIG. 9A and FIG. 9B show agarose gel electrophoresis of RT-PCR amplified products from tissue homogenates targeting POV3 S1 genes. FIG. 9A: S1 segment based RT-PCR on brain tissue homogenates of experimentally infected piglets: Lane M: 1 Kb+ ladder, Lane 1-9: RT-PCR on brain homogenates of experimentally infected piglets, Lane 10—RT-PCR on mock infected brain homogenate, Lane 11: POV3 virus positive control. FIG. 9B: S1 segment based RT-PCR on lung tissue homogenates of experimentally infected piglets: Lane M: 1 Kb+ ladder, Lane 1-9: RT-PCR on brain homogenates of experimentally infected piglets, Lane 10—RT-PCR on mock infected brain homogenate.

FIG. 10A-FIG. 10D depict RT-PCR amplification of S1 segments from POV3 cDNA. FIG. 10A: Amplification plots of cDNA dilutions (10⁻¹ to 10⁻⁶) of the cell culture derived POV3; FIG. 10B: Melt curve analysis of S1 amplified PCR products showing melt peak at 82.5° C.; FIG. 10C: Dissociation curve of S1 amplified PCR products. FIG. 10D: Linearity curve of ct values Vs cDNA dilutions.

FIG. 11A-FIG. 11C show L1 based qRT-PCR amplification of POV3. FIG. 11A: Amplification plots of L1 gene fragment products from the cell culture derived POV3; FIG. 11B: Melt curve analysis of L1 amplified PCR products showing melt peak at 79.5° C.; FIG. 11C: Dissociation curve of L1 amplified PCR products.

FIG. 12A-FIG. 12C represent expression of the recombinant MRV3 al protein in E. coli. SDS-PAGE (FIG. 12A) and Western Blot analysis (FIG. 12B) of the recombinant MRV3 al protein expressed in E. coli. FIG. 12C shows Western blot analysis of the purified MRV3 al protein. M: protein molecular weight standard; S: soluble fraction; P: insoluble fraction; E: elution of the purified recombinant MRV3 al protein. The molecular weight (MW) standard was indicated as kDa. A total of 3 recombinant E. coli clones were analyzed and labeled as “1, 2, and 3”. The S1 protein-specific band has an expected molecular weight of approximately 49 kDa. The smaller S1 protein (S.1s) is produced by the leaky scanning of MRV3 S1 mRNA.

FIG. 13A-FIG. 13D depict Anti-MRV3 IgG antibody level (expressed in OD405 value in the Y-axis) in serum samples of pregnant sows before and after vaccination with an inactivated MRV3 vaccine as detected by an MRV3-specific ELISA. FIG. 13A shows serum sample values from the 6 pregnant sows before vaccination. Pig #9 is a MRV3 antibody-positive pig serum used as a positive control, and pig #230 is MRV3 antibody-negative gnotobiotic pig serum. FIG. 13B shows serum sample values from the two mock-vaccinated sows. (FIG. 13C). Serum samples from the two sows receiving 2 doses of the vaccine. (FIG. 13D). Serum samples from the two sows receiving 3 doses of the vaccine.

FIG. 14A shows the results of binary ethyleneimine (BEI) inactivation kinetics of porcine MRV3 virus at different time points. At 48 hr, BEI completely inactivated all three batches of the MRV3 virus, and thus the 48 hr BEI inactivation was selected to prepare the inactivated vaccine used in the study. FIG. 14B shows the timeline of the sow vaccination and piglet challenge with MRV3 FS03 virus. The pregnant sows, at 56 days of gestation, were vaccinated with an inactivated MRV3 vaccine. Two sows received two doses of the vaccine at 0 and 21 days post-immunization, and another two sows received three doses of the killed vaccines at 0, 21, and 31 days-post-immunization. The conventional piglets were challenged at 4 days of age with MRV3 FS-03 virus. The pigs were necropsied at 4 days post-challenge (dpc).

FIG. 15A-FIG. 15C show viral shedding and body temperature in conventional piglets after challenge with MRV3. FIG. 15A shows the daily body temperature of piglets challenged with MRV3 virus. FIG. 15B shows viral RNA loads in small intestinal content at necropsy. FIG. 15C shows daily fecal viral RNA loads in fecal swab materials. Asterisks (*) indicate statistical difference.

FIG. 16A-FIG. 16B show anti-MRV3 IgG antibodies level in sera of conventional piglets born to pregnant sows that received different vaccination treatment (mock, 2-dose vaccine, and 3-dose vaccine). FIG. 16A shows the results with conventional piglets challenged with MRV-3 virus. FIG. 16B shows the results with conventional piglets challenged with PBS buffer.

FIG. 17A-FIG. 17C show pathogenicity of MRV3 infection in gnotobiotic pigs. FIG. 17A shows daily body temperature of gnotobiotic piglets after MRV3 infection. FIG. 17B shows daily fecal MRV3 RNA shedding in piglets experimentally-infected with MRV3 virus. At 7 days post-challenge (dpc), 6 out of the 8 gnotobiotic piglets were positive for MRV3 RNA in feces. FIG. 17C shows MRV3-specific antibody as detected by ELISA in sera of gnotobiotic piglets at 0 and 7 days post-challenge.

### DETAILED DESCRIPTION OF THE INVENTION

Disclosed herein is a novel porcine orthoreovirus type 3 (POV3) isolated from diarrheic feces of piglets from outbreaks in three states and ring-dried swine blood meal from multiple sources. Genetic and phylogenetic analyses of two POV3 isolates revealed that they are identical but differed significantly from nonpathogenic mammalian orthoreoviruses circulating in the United States. Provided herein are diagnostics and vaccines to identify control and prevent this new infectious agent, including through the detection and inactivation of the virus in porcine blood products.

Despite strict biosecurity and vaccination measures against swine enteric coronavirus, the disease identified by the present inventors has continued to spread to at least 32 states of USA and other countries including Mexico, Peru, Dominican Republic, Canada, Columbia and Ecuador in the Americas and Ukraine with repeated outbreaks. As disclosed herein, the present inventors have demonstrated the association and pathogenicity of porcine Orthoreovirus type 3 (POV3) with these outbreaks in pigs. As used herein the novel virus isolates are also referred to as POV3-VT (Virginia Tech), which includes the isolates F503 and BM100 as well as POV3 strains having a σ1 capsid protein with 98% sequence homology to the σ1 capsid protein (SEQ ID NO: 20) encoded by segment S1 as well as nucleic acids that encode a protein having a 98% sequence homology to the σ1 capsid protein of SEQ ID NO: 20.

As disclosed herein, the present inventors have isolated and characterized a novel porcine POV3 from fecal samples in cases of epidemic piglet diarrhea and have shown that the high pathogenicity of these novel POV3 strains in neonatal pigs leads to lethal enteric disease. We have also isolated these novel POV3 strains from swine blood meal, which is a by-product of the slaughtering industry and is used as a protein source in the diets of livestock. A chloroform extract of blood meal and a virus derived from the same sample caused similar disease in experimental pigs, suggesting blood meal as a source of infection. Indeed, more than 80% of ring-dried blood meal feed supplements were found positive for the novel POV3 virus. Importantly, while the World Organization for Animal Health Office International des Epizooties (OIE) ad hoc group on porcine epidemic diarrhea virus (PEDV) recently concluded that contaminated pig blood products, including spray-dried plasma, are not a likely source of infectious PEDV because spray-drying typically inactivates enveloped coronaviruses. In contrast to PEDV, the novel POV3 virus disclosed herein is particularly heat resistant such that, if present in pig blood products, it will not be property inactivated according to standard procedures.

Our results showed the POV3 isolates are thermostable and trypsin resistant, kill developing chicken embryos, and produce syncytium in BHK-21 cells but not in Vero cells. Fusogenic orthoreoviruses, including MRVs, encode a fusion-associated small transmembrane (FAST) protein that is responsible for syncytiogenesis. However, the POV3 strains identified herein lack this protein but nonetheless produce syncytium in infected BHK-21 cells or intestinal epithelium. The virions were double layered with a mean diameter of 82 nm, in concordance with the reported size for MRVs, but are larger than the reported sizes of 70 to 72 nm for bat orthoreoviruses. Size differences in MRV particle forms, such as virions, intermediate subvirion particles (ISVPs), and core particles, have been reported. Viral factories with paracrystalline arrays of virions in infected Vero cells are an important characteristic of these strains, unlike the tubular viral factories seen in T3D type strains. Our results suggest that POV3 may use intestinal microvilli to release complete virions as arrays in addition to cell lysis.

Deep sequencing analysis of the purified cell culture or developing chicken embryo isolates revealed a novel POV3 sequence. The sequencing data from two selected porcine POV3 isolates (one each from feces and blood meal) revealed a high sequence homology, thus strongly suggesting that blood meal could be a possible mode of transmission along with other undetermined modes. The thermostability of these POV3 strains at 56, 80, and 90° C. for 1 hour lends further credence to this notion. Ring drying of blood meal entails coagulation of blood by heating to 90° C., which may not be sufficient to inactivate these heat-resistant POV3 strains. The current European Union regulation for pig blood products for use in pig feeds (EU 483/2014) requiring treatment at 80° C. and storage for 2 weeks at room temperature to inactivate PEDV appears to be insufficient to inactivate the novel POV3 disclosed herein.

The genome sequences of the 10 segments of the strains disclosed herein, revealed interesting features in a unique and novel combination. For example, they carry specific mutations in σ1 protein that would impart trypsin resistance and neurotropism, in μ2 protein for interferon antagonism, and possessed multiple basic residues in the σ1s protein for hematogenous dissemination. The observed nine unique amino acid substitutions on the μ1 protein may have a role in conferring thermostability to these strains as has been found in associated with thermostability in T3-type strains.

Even though MRVs are not generally common in causing severe disease outbreaks in livestock, several strains of porcine MRVs have been isolated from diarrheic pigs in China and Korea. Similarly, certain MRV3 strains have been reported from bats in Europe suffering from clinical disease and in children with bat origin nonfusogenic MRV3 in Europe. All of these studies and our results confirm that the novel POV3 strains reported here are pathogenic. At necropsy, all infected piglets had accumulation of fluid in the intestine. The reproducibility of severe diarrhea and clinical disease with mortality in experimentally infected piglets with isolated POV3 confirms the pathogenic nature of these strains. Villous blunting is a consistent feature of piglets affected by neonatal diarrhea syndrome. The observed protein casts in the renal tubules and mild hepatic lipidosis could be attributed to the metabolic disorder. The presence of isoleucine at position 249 probably prevented the cleavage of σ1 protein by intestinal luminal proteases, enabling efficient viral growth and migration to other tissues compared to the trypsin-sensitive σ1 protein (threonine at 249) in endemic T3D type strains with attenuated virulence.

Provided herein are diagnostic methods able to detect viral infections and infectious material including animal derived protein supplements. In some embodiments, the proteins expressed by the segments listed in Table 2 are detected. Protein expression can be detected by any suitable method. In some embodiments, proteins are detected by immunohistochemistry. In other embodiments, proteins are detected by their binding to an antibody raised against the protein. Antibody binding is detected by techniques known in the art (e.g., radioimmunoassay, ELISA (enzyme linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In certain embodiments, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

For purposes of ELISA assays for detection of viral antigens, provided herein are useful diagnostic reagents for detecting the POV3 infection using an antibody purified from a natural host such as, for example, by inoculating a pig with the porcine TTV or the immunogenic composition of the invention in an effective immunogenic quantity to produce a viral infection and recovering the antibody from the serum of the infected pig. Alternatively, the antibodies can be raised in experimental animals against the natural or synthetic polypeptides derived or expressed from the amino acid sequences or immunogenic fragments encoded by the nucleotide sequence of the isolated POV3. For example, monoclonal antibodies may be produced according to procedures known in the art that are directed to antigens of the isolated novel POV3.

In other embodiments, POV3 proteins were expressed and used in immunodetection assays to detect the presence of POV3 specific antibodies. In particular, serological testing using POV3-specific hemagglutination-inhibition and ELISA assay provide accurate and simple tools for revealing the association of this novel virus infection with diseases. Assay for detection of antibody to purified or partially purified culture derived vPOV3-VT can also be detected by techniques known in the art (e.g., radioimmunoassay, “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, complement fixation assays, immunofluorescence assays, protein A assays, and immunoelectrophoresis assays, etc.

In other embodiments, molecular assays are employed to detect the presence of minute amounts of the virus in pig populations but also in feed supplements. According to one embodiment of the present invention, real-time PCR using POV3 specific primers is used specifically to detect the presence of U.S. porcine POV3, in feed supplements. In other embodiment, chip based hybridization assays are employed to test multiple lots of feed supplements after PCR application. When detected, the feed supplements can be quarantined and further tested for the presence of live virus. In particular, according to the surprising findings of the present inventors, the POV3 disclosed herein is particularly heat resistant thus allowing live virus to survive heat treatments currently employed to generate ring-dried swine blood meal. Through the diagnostics disclosed herein, methods of treatment of swine blood meal are adapted to provide for complete inactivation of the U.S. porcine MRV3 (“POV3”).

Also provided herein are vaccines for prevention of disease. Such vaccine include killed virus vaccines, live attenuated virus vaccines as well as subunit vaccines. Also included in the scope of the present invention are nucleic acid vaccines. Inoculated pigs are protected from viral infection and associated diseases caused by U.S. porcine POV3 infection. The methods protect pigs in need of protection against viral infection by administering to the pig an immunologically effective amount of a vaccine according to the invention, such as, for example, a vaccine comprising an immunogenic amount of the infectious POV3RNA, a plasmid or viral vector containing an infectious DNA clone of POV3, recombinant POV3 DNA, polypeptide expression products, bacteria-expressed or baculovirus-expressed purified recombinant proteins, etc. Other antigens such as other infectious swine agents and immune stimulants may be given concurrently to the pig to provide a broad spectrum of protection against viral infections.

The vaccines comprise, for example, the infectious viral and molecular nucleic acid clones, cloned POV3 infectious DNA genome segments in suitable plasmids or vectors, avirulent live virus, inactivated virus, expressed recombinant capsid subunit vaccine, etc. in combination with a nontoxic, physiologically acceptable carrier and, optionally, one or more adjuvants. Alternatively, DNA derived from the RNA of segments of the isolated POV3 that encode one or more capsid proteins may be inserted into live vectors, such as a poxvirus or an adenovirus and used as a vaccine.

Adjuvants, which may be administered in conjunction with vaccines of the present invention, are substances that increases the immunological response of the pig to the vaccine. The adjuvant may be administered at the same time and at the same site as the vaccine, or at a different time, for example, as a booster. Adjuvants also may advantageously be administered to the pig in a manner or at a site different from the manner or site in which the vaccine is administered. Suitable adjuvants include, but are not limited to, aluminum hydroxide (alum), immunostimulating complexes (ISCOMS), non-ionic block polymers or copolymers, cytokines, saponins, monophosphoryl lipid A (MLA), muramyl dipeptides (MDP) and the like. Other suitable adjuvants include, for example, aluminum potassium sulfate, heat-labile or heat-stable enterotoxin isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

The new vaccines of this invention are not restricted to any particular type or method of preparation. The cloned viral vaccines include, but are not limited to, infectious DNA vaccines (i.e., using plasmids, vectors or other conventional carriers to directly inject DNA into pigs), live vaccines, modified live vaccines, inactivated vaccines, subunit vaccines, attenuated vaccines, genetically engineered vaccines, etc. These vaccines are prepared by standard methods known in the art.

Additional genetically engineered vaccines, which are desirable in the present invention, are produced by techniques known in the art. Such techniques involve, but are not limited to, further manipulation of recombinant DNA, modification of or substitutions to the amino acid sequences of the recombinant proteins and the like

Genetically engineered vaccines based on recombinant DNA technology are made, for instance, by identifying alternative portions of the viral gene encoding proteins responsible for inducing a stronger immune or protective response in pigs (e.g., proteins derived from unique portions of the novel virus as disclosed herein, etc.). Such identified genes or immuno-dominant fragments can be cloned into standard protein expression vectors, such as the baculovirus vector, and used to infect appropriate host cells (see, for example, O'Reilly et al., “Baculovirus Expression Vectors: A Lab Manual,” Freeman & Co., 1992). The host cells are cultured, thus expressing the desired vaccine proteins, which can be purified to the desired extent and formulated into a suitable vaccine product. In one embodiment, the recombinant subunit vaccines are based on bacteria-expressed or baculovirus-expressed capsid proteins of the novel POV3 strains disclosed herein.

If the clones retain any undesirable natural abilities of causing disease, it is also possible to pinpoint the nucleotide sequences in the viral genome responsible for any residual virulence, and genetically engineer the virus avirulent through, for example, site-directed mutagenesis. Site-directed mutagenesis is able to add, delete or change one or more nucleotides (see, for instance, Zoller et al., DNA 3:479-488, 1984). An oligonucleotide is synthesized containing the desired mutation and annealed to a portion of single stranded viral DNA. The hybrid molecule, which results from that procedure, is employed to transform bacteria. Then double-stranded DNA, which is isolated containing the appropriate mutation, is used to produce full-length DNA by ligation to a restriction fragment of the latter that is subsequently transfected into a suitable cell culture. Ligation of the genome into the suitable vector for transfer may be accomplished through any standard technique known to those of ordinary skill in the art. Transfection of the vector into host cells for the production of viral progeny may be done using any of the conventional methods such as calcium-phosphate or DEAE-dextran mediated transfection, electroporation, protoplast fusion and other well-known techniques (e.g., Sambrook et al., “Molecular Cloning: A Laboratory Manual,” Cold Spring Harbor Laboratory Press, 1989). The cloned virus then exhibits the desired mutation.

Immunologically effective amounts of the vaccines of the present invention are administered to pigs in need of protection against viral infection. The immunologically effective amount or the immunogenic amount that inoculates the pig can be easily determined or readily titrated by routine testing. An effective amount is one in which a sufficient immunological response to the vaccine is attained to protect the pig exposed to POV3. Preferably, the pig is protected to an extent in which one to all of the adverse physiological symptoms or effects of the viral disease are significantly reduced, ameliorated or totally prevented.

The vaccine may be administered in a single dose or in repeated doses. Dosages may range, for example, from about 1 microgram to about 1,000 micrograms of the plasmid DNA containing an infectious chimeric DNA genome (dependent upon the concentration of the immuno-active component of the vaccine), but should not contain an amount of virus-based antigen sufficient to result in an adverse reaction or physiological symptoms of viral infection. Methods are known in the art for determining or titrating suitable dosages of active antigenic agent to find minimal effective dosages based on the weight of the pig, concentration of the antigen and other typical factors. In certain embodiments, the infectious viral DNA clone is used as a vaccine, or a live infectious virus can be generated in vitro and then the live virus is used as a vaccine. In that case, from about 50 to about 10,000 of the 50% tissue culture infective dose (TCID₅₀) of live virus, for example, can be given to a pig.

The advantages of live vaccines are that all possible immune responses are activated in the recipient of the vaccine, including systemic, local, humoral and cell-mediated immune responses. The disadvantages of live virus vaccines, which may outweigh the advantages, lie in the potential for contamination with live adventitious viral agents or the risk that the virus may revert to virulence in the field.

To prepare inactivated virus vaccines, for instance, the virus propagation and virus production can occur in cultured porcine cell lines such as, without limitation PK-15 cells as well as BHK-21 cells, Vero cells, etc. Virus inactivation is then optimized by protocols generally known to those of ordinary skill in the art or, preferably, by the methods described herein. Inactivated virus vaccines may be prepared by treating the virus with inactivating agents such as formalin or hydrophobic solvents, acids, etc., by irradiation with ultraviolet light or X-rays, by heating, etc. Inactivation is conducted in manners understood in the art. For example, in chemical inactivation, a suitable virus sample or serum sample containing the virus is treated for a sufficient length of time with a sufficient amount or concentration of inactivating agent at a sufficiently high (or low, depending on the inactivating agent) temperature or pH to inactivate the virus. Inactivation by heating is conducted at a temperature and for a length of time sufficient to inactivate the virus, considering the particular heat stability of the virus as disclosed herein. Inactivation by irradiation is conducted using a wavelength of light or other energy source for a length of time sufficient to inactivate the virus. The virus is considered inactivated if it is unable to infect a cell susceptible to infection.

Attenuated vaccines are prepared by serial passage in a host that affects the virulence of the virus in pigs such that the virus is able to replicate in the pig and generate a full immune response without causing significant morbidity. For instance, attenuated viruses may be prepared by the technique of the present invention which involves the novel serial passage through embryonated chicken eggs.

The preparation of subunit vaccines typically differs from the preparation of a modified live vaccine or an inactivated vaccine. Prior to preparation of a subunit vaccine, the protective or antigenic components of the vaccine must be identified. DNA encoding the antigenic components are cloned and expressed in and purified from bacterial hosts such as E. coli, or other expression systems, such as baculovirus expression systems, for use as subunit recombinant capsid vaccines. Such protective or antigenic components include certain amino acid segments or fragments of the viral capsid proteins which raise a particularly strong protective or immunological response in pigs; single or multiple viral capsid proteins themselves, oligomers thereof, and higher-order associations of the viral capsid proteins which form virus substructures or identifiable parts or units of such substructures; oligoglycosides, glycolipids or glycoproteins present on or near the surface of the virus or in viral substructures such as the lipoproteins or lipid groups associated with the virus, etc. These immunogenic components are readily identified by methods known in the art. Once identified, the protective or antigenic portions of the virus (i.e., the “subunit”) are subsequently purified and/or cloned by procedures known in the art.

If the subunit vaccine is produced through recombinant genetic techniques, expression of the cloned subunit genes, for example, may be expressed by the method provided above, and may also be optimized by methods known to those in the art (see, for example, Maniatis et al., “Molecular Cloning: A Laboratory Manual,” Cold Spring Harbor Laboratory, Cold Spring Harbor, Mass. (1989)).

Genetically engineered vaccines, which are also desirable in the present invention, are produced by techniques known in the art. Such techniques involve, but are not limited to, the use of RNA, recombinant DNA, recombinant proteins, live viruses and the like. Genetically engineered proteins, useful in vaccines, for instance, may be expressed in insect cells, yeast cells or mammalian cells. The genetically engineered proteins, which may be purified or isolated by conventional methods, can be directly inoculated into a porcine or mammalian species to confer protection.

For baculovirus expression, an insect cell line (such as sf9, sf21, or HIGH-FIVE) is transformed with a transfer vector containing genetic material obtained from the virus that encodes one or more of the unique and immuno-dominant proteins of the virus.

The vaccine can be administered in a single dose or in repeated doses. Dosages may contain, for example, from 1 to 1,000 micrograms of virus-based antigen (dependent upon the concentration of the immuno-active component of the vaccine), but should not contain an amount of virus-based antigen sufficient to result in an adverse reaction or physiological symptoms of viral infection. Methods are known in the art for determining or titrating suitable dosages of active antigenic agent based on the weight of the bird or mammal, concentration of the antigen and other typical factors. Desirably, the vaccine is administered directly to a porcine or other mammalian species not yet exposed to the virus. The vaccine can conveniently be administered orally, intrabuccally, intranasally, transdermally, parenterally, etc. The parenteral route of administration includes, but is not limited to, intramuscular, intravenous, intraperitoneal and subcutaneous routes.

When administered as a liquid, the present vaccine may be prepared in the form of an aqueous solution, a syrup, an elixir, a tincture and the like. Such formulations are known in the art and are typically prepared by dissolution of the antigen and other typical additives in the appropriate carrier or solvent systems. Suitable carriers or solvents include, but are not limited to, water, saline, ethanol, ethylene glycol, glycerol, etc. Typical additives are, for example, certified dyes, flavors, sweeteners and antimicrobial preservatives such as thimerosal (sodium ethylmercurithiosalicylate). Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Liquid formulations also may include suspensions and emulsions which contain suspending or emulsifying agents in combination with other standard co-formulants. These types of liquid formulations may be prepared by conventional methods. Suspensions, for example, may be prepared using a colloid mill. Emulsions, for example, may be prepared using a homogenizer.

Parenteral formulations, designed for injection into body fluid systems, require proper isotonicity and pH buffering to the corresponding levels of mammalian body fluids. Isotonicity can be appropriately adjusted with sodium chloride and other salts as needed. Suitable solvents, such as ethanol or propylene glycol, can be used to increase the solubility of the ingredients in the formulation and the stability of the liquid preparation. Further additives which can be employed in the present vaccine include, but are not limited to, dextrose, conventional antioxidants and conventional chelating agents such as ethylenediamine tetraacetic acid (EDTA). Parenteral dosage forms must also be sterilized prior to use.

While the making and using of various embodiments of the present invention are discussed in detail below, it should be appreciated that the present invention provides many applicable inventive concepts which can be employed in a wide variety of specific contexts. The specific embodiments discussed herein are merely illustrative of specific ways to make and use the invention and do not delimit the scope of the invention.

The following examples are include for the sake of completeness of disclosure and to illustrate the methods of making the compositions and composites of the present invention as well as to present certain characteristics of the compositions. In no way are these examples intended to limit the scope or teaching of this disclosure.

### Example 1

### Isolation of a Novel MRV3 from Diarrheic Feces of Pigs and Ring Dried Swine Blood Meal

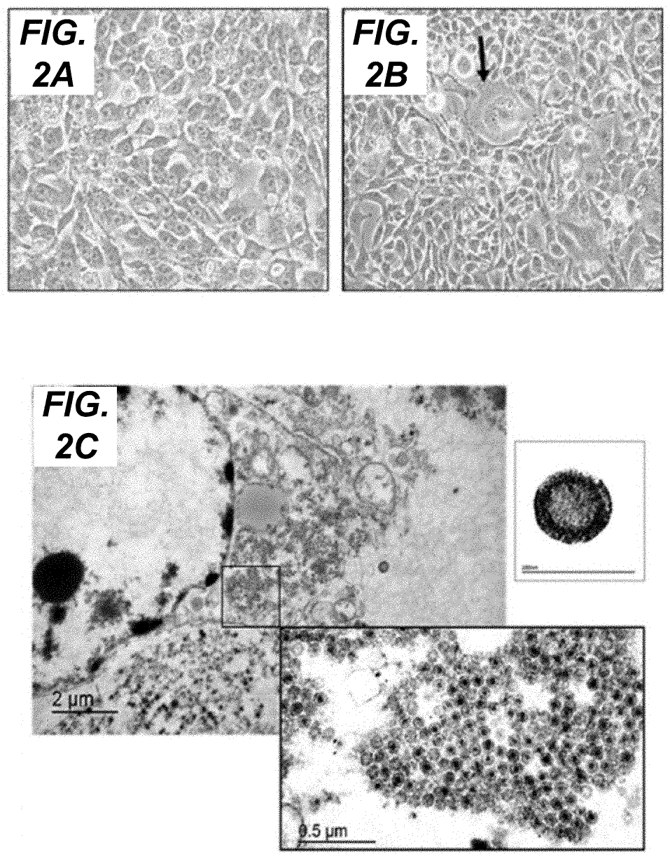

Nine out of 11 ring-dried swine blood meal (RDSB) samples from different manufacturing sources (82%) and 18 out of 48 fecal samples (37%) from neonatal pigs from farms with epidemic diarrhea outbreaks in North Carolina, Minnesota, and Iowa amplified a 326-bp S1 fragment with orthoreovirus group-specific primers. Among the 18 orthoreovirus positive fecal samples, 11 samples were further sequence verified using MRV3-S1 gene-specific primers amplifying a 424-bp fragment. CPE including syncytium formation and rounding of individual cells, were evident at 48 h postinfection (hpi) in BHK-21 cells inoculated with chloroform-extracted samples of feces and blood meal (FIG. 2A-B). The infected cell monolayers were completely detached by 72 to 96 hpi. Developing chicken embryos died 2 to 5 days postinoculation (dpi) after inoculation by the chorioallantoic membrane (CAM) route. Infected chicken embryos showed hemorrhages (“cherry red appearance”) on the body and/or stunted growth (“dwarfing”). MRV3 antigen was detected in infected BHK-21 cells using monoclonal antibody clone 2Q2048 against a MRV3 al protein. The virus isolates from infected BHK-21 cells or chicken embryos were further confirmed as an MRV3 by reverse transcription-PCR (RT-PCR) and sequencing. Eight virus isolates were obtained, and two representative isolates (T3/Swine/FS03/USA/2014 and T3/Swine/BM100/USA/2014) were used for further studies.

To determine whether normal, healthy pigs harbor orthoreoviruses, 36 samples of feces and matched samples of plasma from different states (Indiana, Ohio, Iowa, and Illinois) were obtained from farms with or without a PEDV outbreak. Six samples of feces and plasma each were obtained from uninfected farms in Indiana and Ohio, 12 samples of feces and plasma each were obtained from a farm in Illinois collected 6 weeks post-epidemic diarrhea, and 12 samples of feces and plasma each were obtained from a farm in Iowa collected 6-month post-epidemic diarrhea. None of these samples were found to be positive for orthoreovirus by RT-PCR. Furthermore, chloroform extracts of feces from a few randomly selected MRV3-negative samples were blindly passaged twice on BHK-21 cells, and no CPE was observed.

Viral RNA Isolation.

Viral RNA was isolated from fecal and ring dried swine blood meal samples using the QIAmp RNA kit (Qiagen, United States), and reverse transcription-PCR (RT-PCR) was performed using MRV3-S1 gene-specific primers. The following MRV3 S1 segment specific primers were used (D. Lelli et al., Identification of Mammalian orthoreovirus type 3 in Italian bats. Zoonoses and public health 60, 84-92 (2013)):

SEQ ID NO: 1
S1 Fwd: 5′-338 TGG GAC AAC TTG AGA CAG GA 357-3′,
and
SEQ ID NO: 2
S1 Rev: 5′-644 CTG AAG TCC ACC RTT TTG WA 663-3′,
R = A/G, W = A/T.

The amplified PCR products were analyzed by electrophoresis on a 1.5% (wt/vol) agarose gel, and the PCR products were purified and directly sequenced.

Virus Isolation.

Virus isolation was performed on RT-PCR-positive fecal and blood meal samples. Chloroform extracts of a 20% fecal suspension and 10% ring-dried blood meal samples were filtered through 0.2-μm-pore membrane filters (Millipore, United States) and inoculated into 9 to 11 day old, specific-pathogen-free (SPF), developing chicken embryos (via the chorioallantoic membrane [CAM] route) and BHK-21 cells. Embryos and cells were incubated at 37° C. for 5 days and monitored daily for mortality and cytopathic effects (CPE), respectively. At 5 days postinfection (dpi), allantoic fluid and CAM were harvested from eggs, and the cell culture supernatant was collected from BHK-21 cultures, chloroform extracted, and further passaged in SPF chicken embryos or BHK-21 cells, respectively. Viral RNA was detected by RT-PCR using MRV3 S1 segment-specific primers. Amplified MRV3-S1 PCR products were sequenced to confirm the viral genome. The virus isolates obtained from BHK-21 cells were further confirmed using an indirect immunofluorescence assay (IFA), employing a mouse monoclonal antibody directed against type 3 orthoreovirus al protein (clone 2Q2048; Abcam, United States).

Virus Purification.

BHK-21 cell monolayers grown in T-175 flasks were infected with the POV3 isolates at a multiplicity of infection (MOI) of 0.1 in Dulbecco's modified Eagle's medium (DMEM) containing 1% fetal calf serum (FCS). The cells were harvested at 3 dpi and subjected to three freeze-thaw cycles. The cellular debris was clarified by centrifugation at 3,700×g at 4° C. Crude virus was pelleted from the clarified supernatant by ultracentrifugation at 66,000×g for 2 h using an SW-28 rotor (Beckman Coulter, US). The virus pellet was resuspended in 1 ml TN buffer (20 mM Tris, 400 mM NaCl, 0.01% N-lauryl sarcosine [pH 7.4]). The virus suspension was then layered onto a 15 to 45% (wt/vol) discontinuous sucrose gradient and centrifuged at 92,300×g for 2 h at 4° C. using an SW-41 Ti swing-out rotor (Beckman Coulter, US). The virus band at the interface was collected and used for characterization and genomic studies.

### Example 2

### Morphology and Biological Characteristics

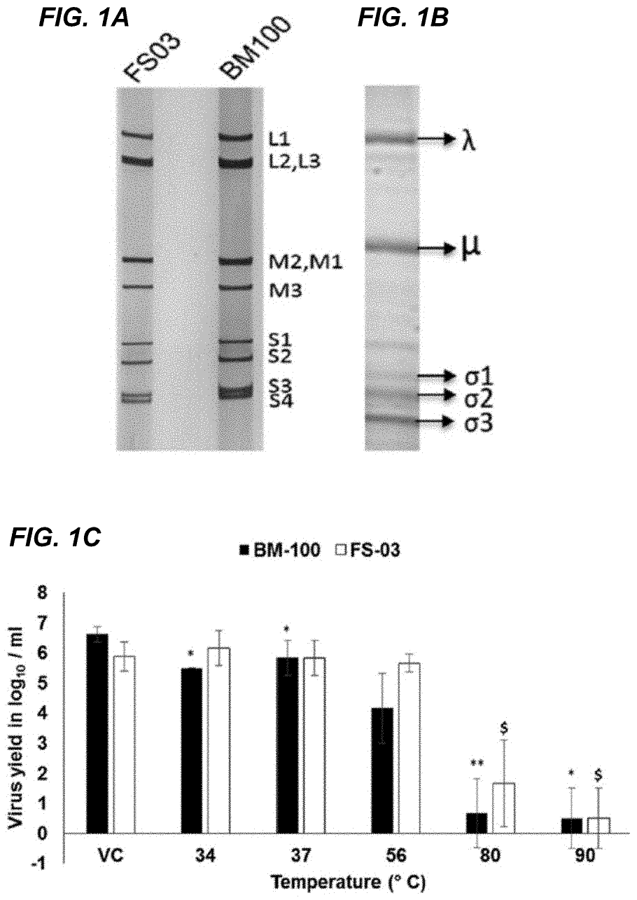

The novel porcine orthoreovirus is unique in morphology and biological characteristics. Genomic RNA from sucrose density gradient-purified virions was resistant to 51 nuclease treatment, confirming the double-stranded nature of the viral genome. SDS-PAGE indicated that the viral genome consists of 10 segments (FIG. 1A). The protein profile of the viruses was consistent with λ, μ, and σ proteins and their subclasses (FIG. 1B). The virions were stable at 56° C. without significant loss of infectivity and remained viable after exposure to 80 or 90° C. for 1 h (FIG. 1C). Transmission electron microscopy (TEM) analysis of negatively stained virions revealed icosahedral, nonenveloped, double-layered uniform sized particles reminiscent of members of the family Reoviridae. (FIG. 2C).

In infected Vero cells, the presence of paracrystalline arrays of virus particles free of organelles and viral factories in the cytoplasm was evident. The mean diameter of the virus particles was 82 nm (FIG. 2C inset), with particle sizes ranging from 80 to 85 nm. The POV3 isolates (FS03 and BM100) replicated efficiently in BHK-21 cells, with a mean tissue culture infective dose (TCID₅₀) of 6.7 log₁₀/ml. Virus infectivity to BHK-21 cells increased after treatment with TPCK trypsin (6.7 to 7.7 log 10/ml), suggesting trypsin resistance. The POV3 strains were able to hemagglutinate swine erythrocytes, and this property could be specifically inhibited with MRV3 anti-σ1 monoclonal antibody.

Virus Characterization.

Hemagglutination (HA) and hemagglutination inhibition (HI) assays were performed. Briefly, the viruses were serially diluted in 50 μl of phosphate-buffered saline (PBS [pH 7.4]) in 96-well V-bottom microtiter plates (Corning-Costar, US) followed by 50μl of 1% pig erythrocytes (Lampire Biological Laboratories, US). The plates were incubated for 2 h at 37° C. to record the HA titer. The HI assay was performed using mouse monoclonal antibody directed against type 3 orthoreovirus 1 protein (clone 2Q2048; Abcam, US) and 4 HA units of the virus. The HI assay plates were incubated initially at 37° C. for 1 h and then at 4° C. overnight before scoring. For electron microscopy, ultrathin sections of virus-infected BHK-21 cells (3 dpi), intestines of experimentally infected pigs, or purified virions were placed on Formvar-carbon-coated electron microscope grids and negatively stained with 2% (wt/vol) uranyl acetate or 1% sodium phosphotungstic acid for 30 s. The specimens were then examined in a JEOL 1400 transmission electron microscope (JEOL, US) at an accelerating voltage of 80 kVA.

To determine the temperature sensitivity, the virus strains were subjected to five different temperature treatments at 34, 37, 56, 80, and 90° C. for 1 h. Serial dilution of the virus was then made in DMEM, which was then titrated for infectivity in BHK-21 cells. For trypsin sensitivity, virus was incubated with 1 μg/ml tosyl phenylalanyl chloromethyl ketone (TPCK) trypsin in DMEM for 1 h at 37° C. and titrated for infectivity in BHK-21 cells. To demonstrate the double-stranded nature of the viral genome, total RNA extracted from purified virions was subjected to 51 nuclease digestion and 7.5% SDS-PAGE and silver nitrate staining. For protein profiling, the purified virus was denatured in protein sample buffer and analyzed by standard 7.5% SDS-PAGE and Coomassie blue staining.

### Example 3

### Virulence Associated Mutations

Deep sequencing (MiSeq) of purified viral RNAs from two selected POV3 isolates (F S03 from a pig fecal sample and BM100 from a porcine blood meal) confirmed their genomic identity with MRV3. No other contaminating viral sequences were detected in the deep sequence data. The high level of sequence identity between FS03 and BM100 sequences validated our immunofluorescence, gel electrophoresis and virus protein profile data. The total length of the porcine orthoreovirus genome is 23,561 nucleotides (nt). The two porcine isolates have consensus genome termini at the 5′ and 3′ ends similar to other MRVs. The 5′ untranslated region (UTR) ranged in length from 12 to 31 nt, and the 3′ UTR ranged in length from 32 to 80 nt, with variations from prototype MRV3 T3D (Table 1). The 5′ UTRs of both POV3 FS03 and BM100 have a 6-nt deletion in L1 and a 1-nt deletion in each of the L2 and S4 segments. In addition, a deletion of 3 nt in the M2 segment open reading frame (ORF) was noticed. The genome of these novel viruses contains reassorted gene segments from other MRVs.

TABLE 1
U.S. porcine orthoreovirus strains (“POV3”)show altered UTRs

| | | 5′ End | ORF/Protein | | | 3′ End | | |
|---|---|---|---|---|---|---|---|---|
| | Size | Terminal | UTR | | Size | | | Terminal |
| Segment | (bp) | Sequence^{a} | (bp) | Region | (aa) | Class | (bP) | Sequence^{a} |
| L1 | 3,854 | GCUACA | 18 | 19-3822 | 1,267 | λ3 | 32 | ACUCAUC |
| L2 | 3,915 | GCUAUU | 12 | 13-3882 | 1,289 | λ2 | 33 | AUUCAUC |
| L3 | 3,901 | GCUAAU | 13 | 14-3841 | 1,275 | λ1 | 60 | AUUCAUC |
| M1 | 2,304 | GCUAUU | 13 | 14-2224 | 736 | μ2 | 80 | CUUCAUC |
| M2 | 2,205 | UGCUAAU | 30 | 31-2157 | 708 | μ1 | 48 | AUCAUCA |
| M3 | 2,241 | GCUAAA | 18 | 19-2184 | 721 | μNS | 57 | AUUCAUC |
| S1 | 1,416 | UGCUAUU | 14 | 15-1382, | 455, | σ1, σ1s | 34 | CACUUAA |
| | | | | 73-435 | 120 | | | |
| S2 | 1,331 | GCUAUU | 18 | 19-1275 | 418 | σ2 | 56 | ACUGACC |
| S3 | 1,198 | GCUAAA | 27 | 28-1128 | 366 | σNS | 70 | AAUCAUC |
| S4 | 1,196 | GCUAUU | 31 | 32-1129 | 365 | σ3, σ3a, | 67 | AUUCAUC |
| | | | | | | σ3b | | |

^{a}The 5′ and 3′ untranslated regions (UTRs) of U.S. porcine strains F503 and BM100 show mutations on the M2, S1, and S2 segments. The conserved terminal sequences are shown in boldface, and mutations are italicized.

Predicted functions of different proteins encoded by the 10 segments analogous to known members of the Orthoreovirus genus are shown in Table 2 below:

TABLE 2
Orthoreovirus protein functions

| Genome | Protein | Size | |
|---|---|---|---|
| Segment | Class | (aa) | Protein Function |
| L1 | λ3 | 1,267 | Core protein, RNA-dependent |
| | | | RNA polymerase |
| L2 | λ2 | 1,289 | Core protein; Guanyltransferase, |
| | | | methyltransferase |
| L3 | λ1 | 1,275 | RNA binding, NTPase, helicase, RNA |
| | | | triphosphatase |
| M1 | μ2 | 736 | Core Protein, Binds RNA NTPase |
| M2 | μ1 | 708 | Outer capsid protein, Cell |
| | | | penetration, transcriptase activation |
| M3 | μNS | 721 | Unknown |
| S1 | σ1, σ1s | 455, | Outer capsid protein, Cell attachment, |
| | | 120 | hemagglutinin, type-specific antigen |
| S2 | σ2 | 418 | Inner capsid structural protein, |
| | | | Binds dsRNA |
| S3 | σNS | 366 | Inclusion formation, binds ssRNA |
| S4 | σ3, σ3a, σ3b | 365 | Binds dsRNA |

The deduced amino acid sequences of POV3 FS03 and BM100 are homologous except for the σ1 protein, with 1 amino acid (aa) change between them. The percentage of homology of each of the different proteins coded by these two viruses with prototype MRV 1-4 is provided in Table 3 below:

TABLE 3
Percentage of Homology with Prototype MRV 1-4

| | | | | | | MRV3 |
|---|---|---|---|---|---|---|
| Segment/ | U.S. | MRV1 | MRV2 | MRV3 | MRV4 | Bat/ |
| Protein | Isolates | T1/L | T2/J | T3/D | T4/Ndelle | Germany |
| L1/λ3 | FS 03 | 98% | 92% | 98% | 97% | 98% |
| | BM100 | 98% | 92% | 98% | 97% | 98% |
| L2/λ2 | FS 03 | 98% | 87% | 92% | NA | 92% |
| | BM100 | 99% | 87% | 92% | NA | 92% |
| L3/λ1 | FS 03 | 99% | 95% | 99% | NA | 98% |
| | BM100 | 99% | 96% | 99% | NA | 98% |
| M1/μ2 | FS 03 | 97% | 80% | 96% | NA | 94% |
| | BM100 | 98% | 80% | 96% | NA | 94% |
| M2/μ1 | FS 03 | 98% | 97% | 97% | 97% | 97% |
| | BM100 | 98% | 97% | 97% | 97% | 97% |
| M3/μNS | FS 03 | 95% | 95% | 96% | NA | 95% |
| | BM100 | 95% | 95% | 96% | NA | 95% |
| S1/σ1 | FS 03 | 28% | 27% | 85% | 65% | 91% |
| | BM100 | 29% | 27% | 85% | 65% | 91% |
| S2/σ2 | FS 03 | 98% | 93% | 98% | 97% | 98% |
| | BM100 | 98% | 94% | 98% | 97% | 98% |
| S3/σNS | FS 03 | 98% | 86% | 98% | NA | 99% |
| | BM100 | 98% | 87% | 98% | NA | 99% |
| S4/σ3 | FS 03 | 86% | 87% | 85% | 85% | 88% |
| | BM100 | 86% | 87% | 85% | 85% | 88% |

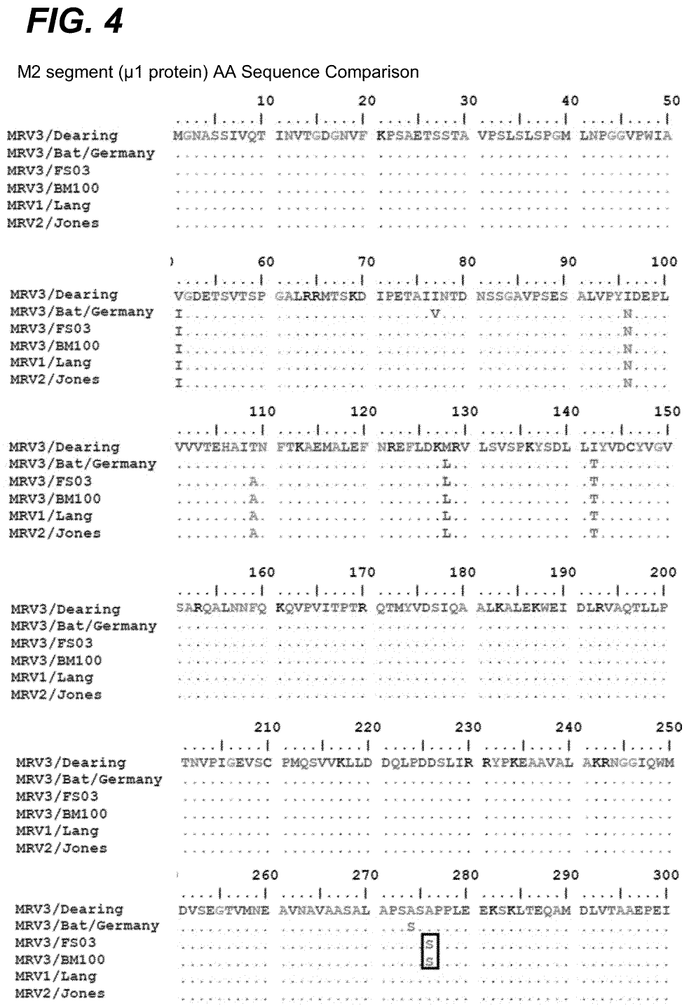

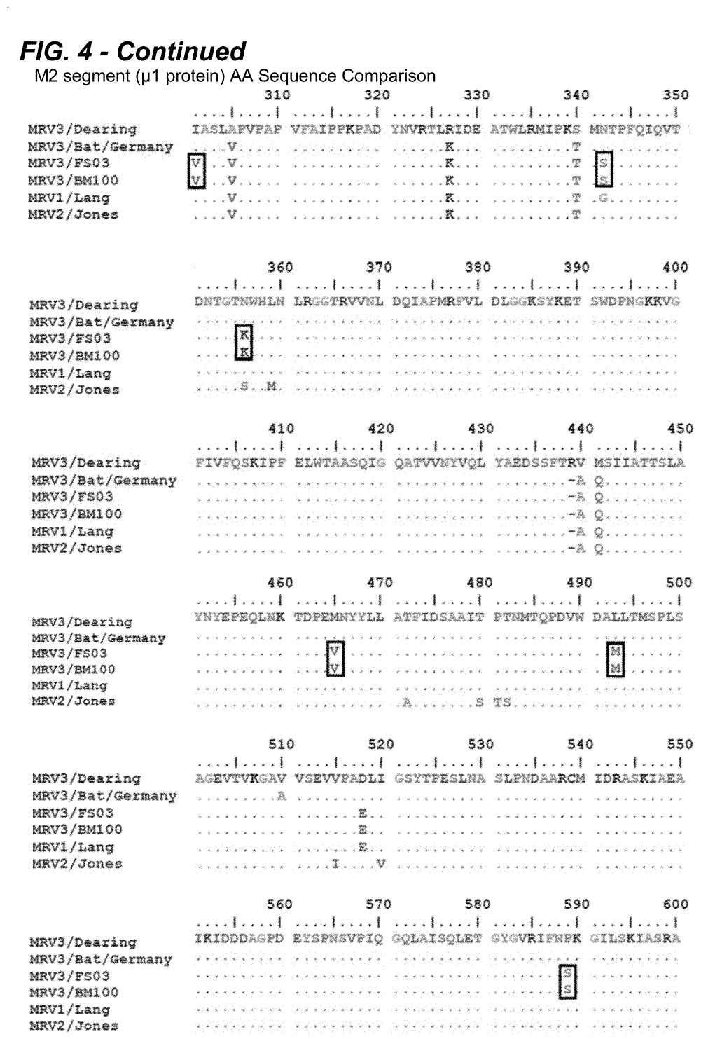

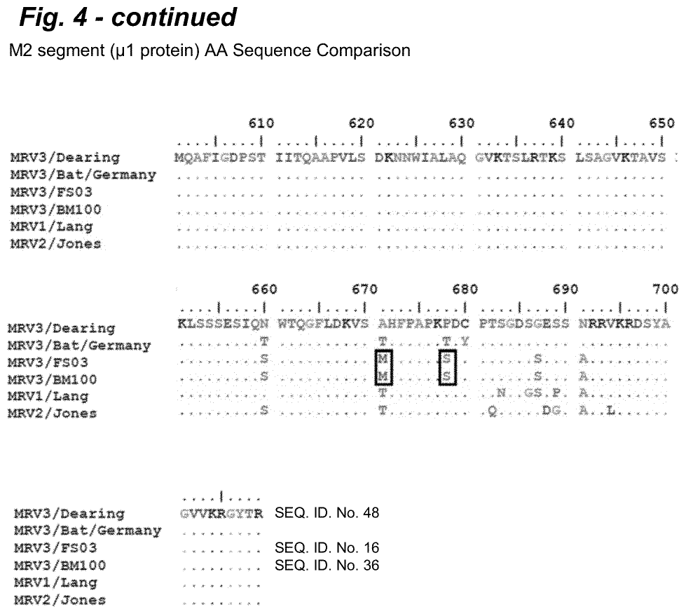

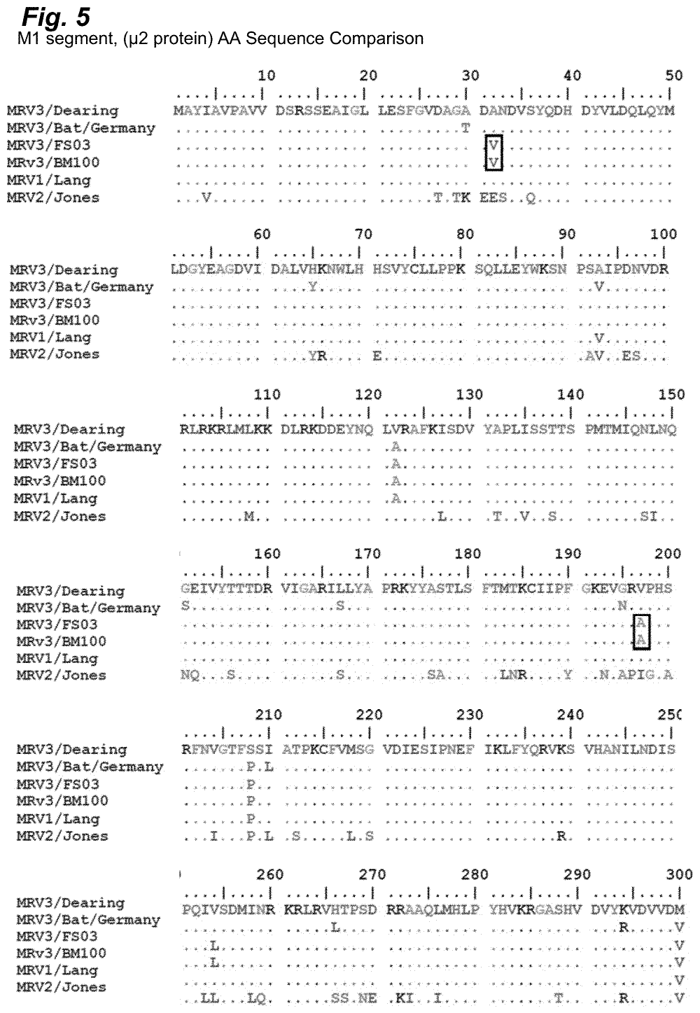

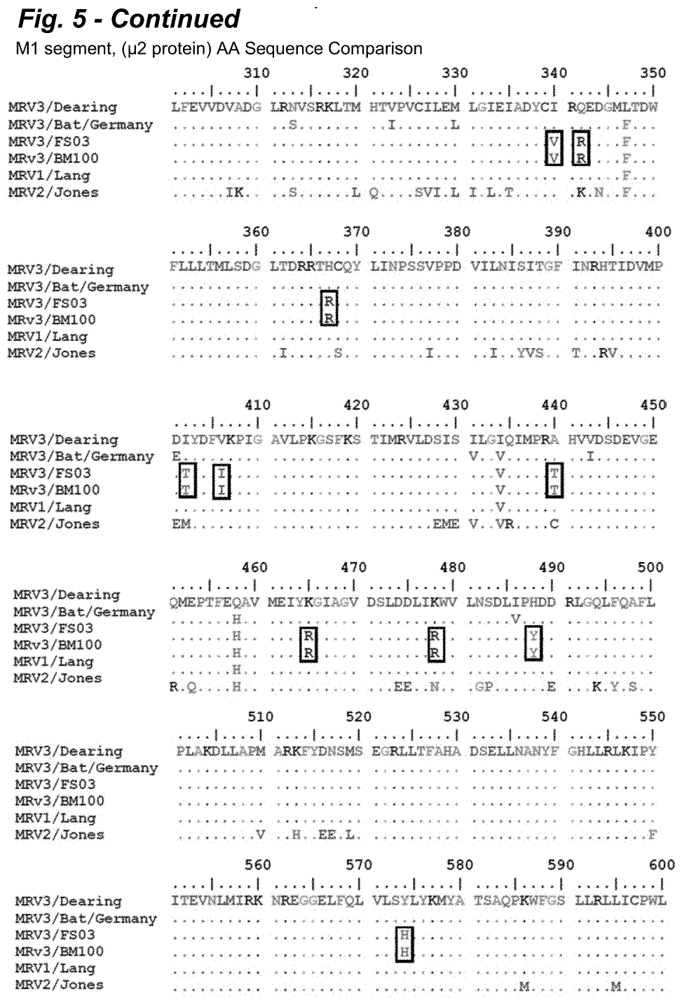

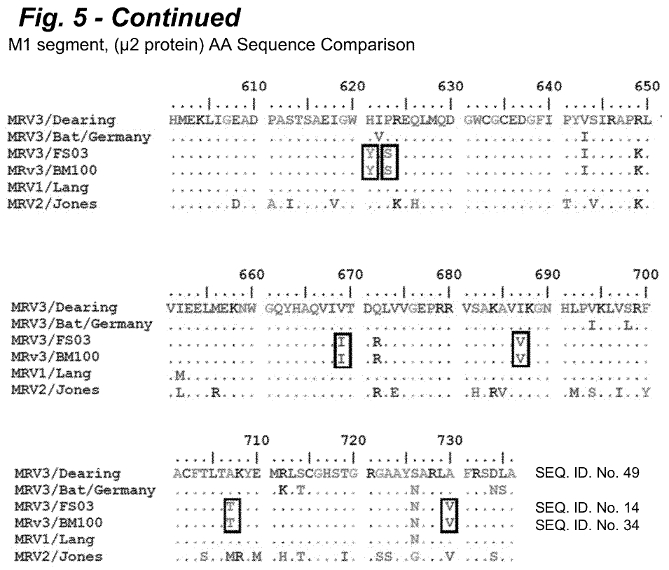

On comparison of the deduced amino acids, it appears that with proteins of the L class segment, λ2 protein was homologous to MRV1, while the λ1 and λ3 proteins were highly similar to the MRV 1 and 3 prototypes, T1-Lang (T1L) and T3/Dearing (T3D), respectively. In M class proteins, only μNS was identical to T3D, while μ1 and μ2 were identical to T1L. As shown in FIG. 4, the sequence alignment of the M2 segment encoded μ1 protein indicated 6 amino acid substitutions that were unique to these isolates in comparison to the T3/Dearing (SEQ ID NO: 48), T3/Bat/Germany, T1L, and T2J isolates). As shown in FIG. 5, the M1 segment encoded μ2 protein alignment revealed 15 unique amino acid substitutions compared to the T3/Dearing (SEQ ID NO: 49). T3/Bat/Germany, T3D, T1L, and T2J sequences and possessed the 5208P mutation compared to T3D. In the S class proteins, all of them appear to originate from European bat (MRV3) viruses, with 88% to 98% identity at amino acid level.

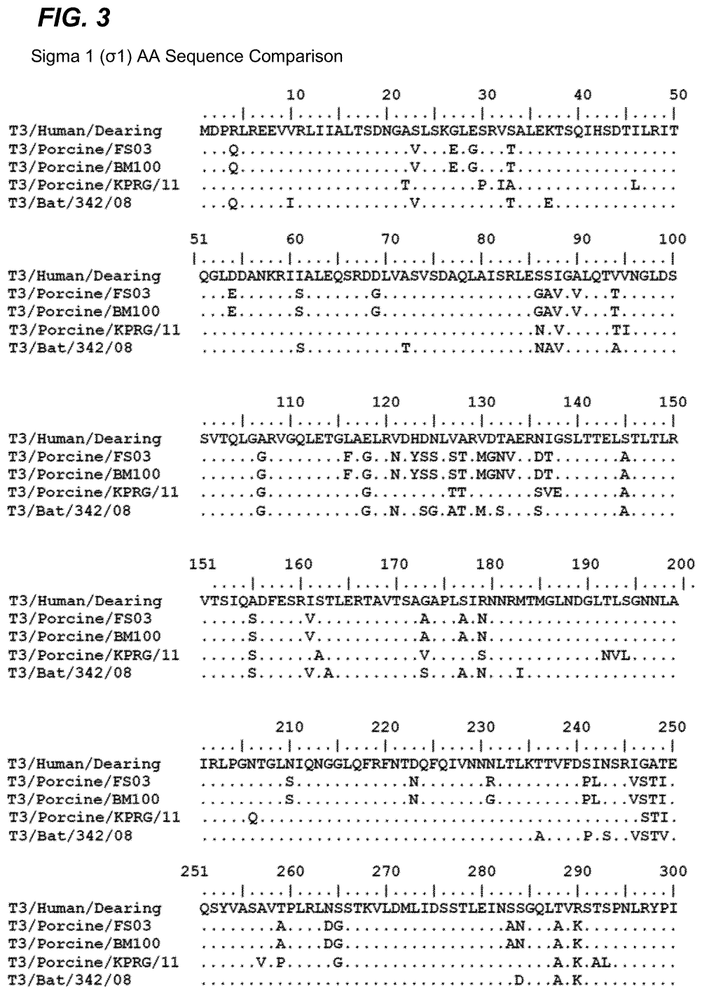

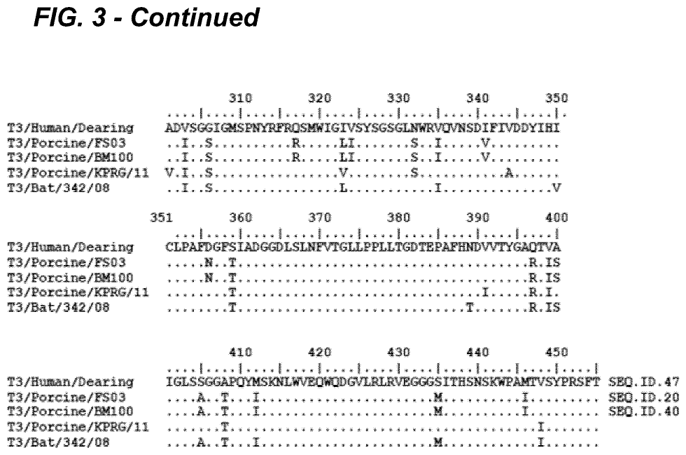

The highest diversity among all proteins was observed for the S1 segment encoded al protein, with closest homology to T3/Bat/Germany virus (91%). Deduced amino acid sequence analysis of al protein revealed that the sialic acid binding domain (NLAIRLP), and protease resistance (249I) and neurotropism (340 D and 419E) residues were conserved in the U.S. porcine orthoreovirus strains. As depicted in FIG. 3, with the alignment based on T3/Dearing (SEQ ID NO: 47), the novel POV3 viruses possessed 31 and 11 unique amino acid substitutions in the σ1 and σ1s proteins in comparison to T3/Bat/Germany and other MRV prototypes. The al s proteins are produced by leaky scanning of the S1 segment. In the leaky scanning phenomena, a weak initiation codon triplet on mRNA may be skipped by the ribosomal subunit in translation initiation. The ribosomal subunit continues scanning to a further initiation codon. The weak initiation codon can be an ACG, or an ATG in a weak Kozak consensus context. Produced mRNAs from leaky scanning may encode several different proteins if the AUG are not in frame, or for proteins with different N-terminus if the AUG are in the same frame.

Deep Sequencing.

The double-stranded RNA (dsRNA) isolated from two purified viruses, FS03 isolated from fecal samples and BM100 isolated from swine ring-dried blood meal, were subjected to NextGen genome sequencing. The NEBNext Ultr directional RNA library prep kit for Illumina (catalog no. e74205; NEB) was used to prepare the RNA library with some modifications. Using a standard protocol, 100 ng of viral RNA was fragmented to 250 nucleotides at 94° C. for 10 min. After adapter ligation, 350- to 375-bp libraries (250- to 275-bp insert) were selected using Pippin Prep (Sage Science, United States). The template molecules with the adapters were enriched by 12 cycles of PCR to create the final library. The generated library was validated using the Agilent 2100 bioanalyzer and quantitated using the Quant-iT dsDNA H.S. kit (Invitrogen) and quantitative PCR (qPCR). Two individually barcoded libraries (FS03 virus with A006-GCCAAT, and BM100 virus with A012-CTTGTA) were pooled and sequenced on Illumina MiSeq. Briefly, the individual libraries were pooled in equimolar amounts, denatured, and loaded onto MiSeq. The pooled library was spiked with 5% phiX and sequenced to 2×250 paired-end reads (PE) on the MiSeq using the MiSeq reagent kit V2 at 500 cycles (MS-102-2003) to generate 24 million PE.

Genome Assembly.

Reference-based mapping and de novo assembly methods were applied to the raw data for assembly into viral genomes. Reference-based mapping was performed against the mammalian orthoreovirus genome by using the CLC Genomics Workbench software (version 7.0.4; CLC Bio, Denmark). The de novo assembly was performed with the following overlap settings: mismatch cost of 2, insert cost of 3, minimum contig length of 1,000 bp, a similarity of 0.8, and a trimming quality score of 0.05. This assembly yielded 3,444 contigs that were annotated according to Gene Ontology terms with the Blast2Go program, which was executed as a plugin of CLC by mapping against the UniprotKB/Swiss-Prot database with a cutoff E value of 1e-05. Furthermore, to determine putative gene descriptions, homology searches were carried out through querying the NCBI database using the tBLASTx algorithm. The de novo-assembled sequences were used to confirm the validity of the reference-based sequence assembly. Both de novo assembly and the reference-based mapping produced identical sequences.

Nucleotide Sequence Accession Numbers.

The complete genome sequences of both viruses FS03 and BM100 are provided herein and have been deposited in GenBank under accession no. KM820744 to KM820763 as shown in Table 4 below. In the Table, the proteins for which alignments are provided in FIGS. 3-5 are highlighted.

TABLE 4
GenBank accession numbers of U.S. porcine orthoreovirus (POV3) isolates and
prototype sequences used

| | MRV3 | MRV | MRV1 | MRV2 | MRV3 Dearing |
|---|---|---|---|---|---|
| Segm't | FS03 | BM100 | T1/L | T2/J | T3D |
| L1 | KM820754, SEQ | KM820744, SEQ | M24734 | M31057 | HM159613 |
| | ID NO: 7, 8 | ID NO: 27, 28 | | | |
| L2 | KM820755, SEQ | KM820745, SEQ | AF378003 | AF378005 | HM159614 |
| | ID NO: 9, 10 | ID NO: 29, 30 | | | |
| L3 | KM820756, SEQ | KM820746, SEQ | AF129820 | AF129821 | HM159615 |
| | ID NO: 11, 12 | ID NO: 31, 32 | | | |
| M1 | KM820757, SEQ | KM820747, SEQ | AF461682 | AF124519 | HM159616, |
| | ID NO: 13, 14 | ID NO: 33, 34 | | | SEQ ID NO: 49 |
| M2 | KM820758, SEQ | KM820748, SEQ | AF490617 | M19355 | HM159617, |
| | ID NO: 15, 16 | ID NO: 35, 36 | | | SEQ ID NO: 48 |
| M3 | KM820759, SEQ | KM820749, SEQ | AF174382 | AF174383 | HM159618 |
| | ID NO: 17, 18 | ID NO: 37, 38 | | | |
| S1 | KM820760, SEQ | KM820750, SEQ | M14779 | M35964 | HM159619, |
| | ID NO: 19, 20 | ID NO: 39, 40 | | | SEQ ID NO: 47 |
| S2 | KM820761, SEQ | KM820751, SEQ | M17578 | L19775 | HM159620 |
| | ID NO: 21, 22 | ID NO: 41, 42 | | | |
| S3 | KM820762, SEQ | KM820752, SEQ | M14325 | M18390 | HM159621 |
| | ID NO: 23, 24 | ID NO: 43, 44 | | | |
| S4 | KM820763, SEQ | KM820753 SEQ | M13139 | DQ318037 | HM159622 |
| | ID NO: 25, 26 | ID NO: 45, 46 | | | |

### Example 4

### The Novel U.S. Porcine Orthoreovirus is Evolutionarily Related to MRV3

Phylogenetic analysis of the FS03 and BM100 POV3 isolates revealed a strong evolutionary relationship with MRV3 strains. The ORFs of the nucleotide sequences of the L1, S1, S2, S3, and S4 segments were used to construct the phylogenetic trees. Based on S1 phylogeny, both isolates were monophyletic with MRV3 of bat origin (FIG. 3) and formed a distinct lineage together with the bat strains under lineage 3, along with the human, bovine, murine, and bat strains with close evolutionary distance to German and Italian bat MRV3 S1 sequences. Phylogenetic analysis of segment S2 indicated that the novel POV3 isolates were monophyletic with the human T3D, T1L, and Chinese porcine T1 strains. The S3 phylogeny indicated that U.S. POV3 strains were closely related to T1L and Chinese pig and European bat MRV3 strains. The topologies of the S4 segment phylogenetic trees revealed that the U.S. porcine MRV3 (POV3) isolates were closely related to Chinese T1 and T3 pig isolates. The L1 segment phylogeny revealed a close relationship to Chinese porcine T3 strains. The sequence diversity of S2, S3, and S4 segments does not correlate with host species, geographic location, or year of isolation, suggesting their origin from different evolutionarily distinct strains from humans, pigs, and bats and as obtained by MRV reassortment in nature

Phylogenetic Analysis.

The nucleotide and deduced amino acid sequences of L1 and S class segments (S1, S2, S3, and S4) were compared with those of other closely related orthoreoviruses using the BioEdit sequence alignment editor software (version 7.0.0; BioEdit, Ibis Biosciences, Carlsbad, Calif.). The phylogenetic evolutionary histories for the virus strains were inferred using the maximum likelihood method based on either JTT w/freq model for the S2, S3, S4, and L1 segments in Mega 6.06 or the Jukes-Cantor evolution model with “WAG” (i.e., Whelan and Goldman model) protein substitution for S1 segment in CLC workbench 7.0.4 after testing for their appropriateness to be the best fit. The bootstrap consensus tree inferred from 1,000 replicates was taken to represent the evolutionary history of the taxa analyzed.

### Example 5

### The Novel U.S. Porcine Orthoreovirus (POV3-VT) is Highly Pathogenic in Pigs

Experimental neonatal pigs were screened for swine deltacoronavirus, PEDV, Kobuvirus, swine transmissible gastroenteritis virus (TGEV), rotavirus, and orthoreoviruses by RT-PCR and found to be negative, except for three pigs that were positive for Kobuvirus, whose pathogenicity is yet to be established. Neonatal pigs orally inoculated with purified viruses F503, BM100, T3/Swine/I03/USA/2014 (103), or a chloroform extract of blood meal 100 (CBM100) developed clinical illness in all infected animals (100%), with loss of physical activity, severe diarrhea, and decrease in body weight. Infected animals had significantly high mean clinical scores compared to the mock-infected group (P<0.01). Piglets infected with FS03 and 103 had the highest clinical scores as early as 1 dpi, which peaked at 3 dpi. Three pigs in the mock-infected group had a slow recovery from parenteral anesthetics, with elevated mean clinical scores for the first 2 days but returned to normal later. Gross lesions, such as catarrhal enteritis and intussusception, were observed in all of the infected animals. The cumulative macroscopic lesion scores of FS03 and 103 were higher than those of other groups on day 4 dpi. Compared to mock-infected pigs, the small intestines of the virus-infected pigs showed mild to severe villous blunting and fusion (crypt/villous ratios of 1:1 to 1:4), occasional villous epithelial syncytial cells, swollen epithelial cells with granular cytoplasm and multifocal necrosis of mucosal epithelium, and round to oval vacuoles in the intestinal epithelial cells. In a few pigs, protein casts in renal tubules, minimal to mild hepatic lipidosis and hepatocellular vacuolar changes, and mild to moderate suppurative bronchopneumonia were also seen.

Ultrastructural examination revealed multinucleated cells with apoptotic nuclei, and in some cells, dark granular bodies resembling stress granules were seen. Viral particles were localized in regions of the cytoplasm that lacked typical cytoplasmic organelles. Large numbers of viral particles egressed by cell lysis or as a string of beads through microvilli from infected villous epithelial cells into the lumen of the intestine. Multinucleated cells with virions egressing through microvilli were evident. Virions disrupt microvilli before release and were still surrounded by the cell membrane of microvilli, and after release were devoid of membranes in the lumen of the intestine.

Virus replication in the intestines and fecal virus shedding in infected pigs were also confirmed by virus isolation in cell culture and by S1-segment-specific RT-PCR. The intestinal contents had POV3 virus in 80% of the infected piglets through RT-PCR, suggesting the virus replication in the intestine is consistent with electron microscopic findings of virus replication within the enterocytes.

Pathogenicity Study in Neonatal Pigs.

All animal studies were performed as approved by the Institutional Animal Care and Use Committee of Virginia Tech (IACUC no. 14-105-CVM, 5 Jun. 2014). Thirty-five 2-day-old piglets, purchased from the Virginia Tech Swine Center, were housed as 7 animals/group in HEPA-filtered level 2 biosecurity facility.

Prior to the start of the experiment, pigs were tested for most common enteric RNA viruses, such as rotavirus, PEDV, swine deltacoronavirus, Kobuvirus, and TGEV, by RT-PCR of the fecal samples using specific primers (primer sequences available upon request). The amplified PCR products were analyzed by electrophoresis on 1.5% (wt/vol) agarose gel.

After acclimatizing for a day, the animals were anesthetized, and 2 ml of 5×10⁵ TCID₅₀/ml of each virus strain or chloroform extract of 10% blood meal suspension (2.5 g ring-dried blood meal) was homogenized in 12.5 ml DMEM to get a 20% solution that was extracted with an equal volume of chloroform. The upper aqueous phase obtained was diluted with an equal volume of DMEM to get a final concentration of 10%, and the piglet was orally inoculated using a 5-ml syringe. Mock-infected animals received 2 ml DMEM orally. The animals were monitored two times a day: rectal temperature, body weight, and clinical scores based on physical appearance, activity, respiratory, gastrointestinal, and systemic signs were recorded on a scale of 0 to 3. Fecal swabs were collected daily and suspended in 1 ml of DMEM containing 10×antibiotic solution (Hy-Clone, United States), mixed vigorously, incubated for 1 h, and stored at −80° C. until tested. At 4 dpi, or when they reached the clinical endpoint, all animals were euthanized. Gross and microscopic lesions were scored by a board-certified veterinary pathologist blind to the experimental groups. The 51 gene-specific RT-PCR was performed to confirm the production of orthoreovirus in the intestine using the intestinal contents of the experimentally infected piglets.

Statistical Analysis.

Summary statistics were calculated to assess the overall quality of the data. Analysis of variance (ANOVA) was used for assessment of the mean clinical score and microscopic lesion scores. The significance level was set for a P value of <0.01 and a 95% confidence interval. Statistical analysis was performed using GraphPad Prism software (version 6.0; Graph Pad Software, Inc., San Diego, Calif.).

### Example 6

### Discrepancy Between HI Titers and Virus Neutralizing Antibodies

To identify the prevalence and geographic distribution of this novel orthoreovirus, a retrospective serological surveillance of 1067 sera samples collected from 24 states during 2014-2015 and 28 sera samples from 2008 was performed. Samples were tested by Hemagglutination Inhibition (HI) of pig erythrocytes with plaque purified porcine Orthoreovirus type 3 (POV3) and virus neutralization (VN) test in BHK21 cells. The prevalence of POV3-specific HI antibodies was very high during 2014-2015 but negative for samples from 2008. The HI titers ranged from 2 to 4096 against POV3 with 88.37% of samples above the cut-off titer of 1:8. High HI antibody titers (2048 and above) were recorded only from swine sera samples collected from Iowa, North Carolina Pennsylvania, Texas, South Dakota, Oklahoma, Montana, Michigan, Georgia and Colorado. There were no significant differences in the HI titers with respect to age (1-56 weeks) of pigs. However, serum neutralization assay on 200 randomly selected samples showed low levels of VN antibodies (<1:10). The prevalence of high titer HI antibodies and low level of VN antibodies has warranted the immediate development of vaccines against this pathogenic POV3, as exemplified herein.

### Example 7

### Killed Porcine Orthoreovirus Vaccine by Binary Ethyleneimine (BEI) Inactivation of Porcine Orthoreovirus

One example of a killed virus vaccine was generated by Binary Ethyleneimine (BEI) Inactivation. The virus strain designated POV3-BM100 was originally isolated from swine ring dried blood meal. The virus was initially propagated in BHK-21 culture three times and was plaque purified. Virus plaque no. 2 was further propagated and amplified twice in BHK-21 cells to make a Master Seed virus. The titer of the virus was determined by TCID₅₀ assay. Cell cultures are grown in Dulbecco's modified minimal essential media (Hyclone DMEM/High Glucose Thermo. USA, cat no: SH30243.02) supplemented with 10% FCS and Hyclone 1× penicillin-Streptomycin solution, Thermo, USA cat no V30010) antibiotic and anti-mycotic solution. The serum concentration was reduced to 1% for a maintenance medium and chymotrypsin was added at a concentration of 1 ug/mL to the maintenance medium to promote virus infectivity. BHK-21 cells grown in T-175 flask at 37° C., 5% CO₂ with 80-90% confluency are used for virus infection. The growth media is removed. The seed virus is thawed on ice. The cell monolayers are washed thrice in sterile PBS. Sufficient virus is added to achieve a minimum multiplicity of infection (MOI) of 0.01. The fluids are harvested along with the cellular material 72 hours after infection, dispensed and frozen at −80° C. The working seed lot of the virus is sonicated or given 3-4 freeze thaw cycles (at −80° C.) to release the intracellular virions to be used for inactivation. The viral suspension is centrifuges at 3000 rpm for 20 min at 4° C. and the supernatant fluids harvested. The titer of the virus before inactivation is determined using TCID₅₀ method or plaque assay in triplicates. Non-frozen porcine orthoreovirus produced as described above can be further inactivated using binary ethyleneimine (BEI).

BEI Inactivation:

BEI is prepared from 0.1M 2-bromo-ethylamine hydrobromide (2-BEA, Acro Organics, USA, Catalogue no 2576-47-8) in solution of 0.2 N NaOH (Sigma, USA) and the BEA solution is treated in water bath at 37° C. for 1 hour for the cyclization reaction that converts BEA to BEI (0.1M BEI stock solution). A solution of 0.1M BEI is further filter sterilized using 0.22 micron syringe filter and used immediately for Virus inactivation. BEI was used at three different concentrations viz 1 mM, 2.5 mM and 5 mM. Samples are harvested to evaluate the inactivation process. Control samples are also retained for comparison (Mock infected cell culture supernatant). Samples are taken using aseptic technique inside the bio-safety cabinet. At the end of each time point (incubation period) 2% v/v of a sterile 1M sodium thiosulfate solution was added to ensure neutralization of the BEI. The neutralized sample is thoroughly mixed on a vortex mixer and stored at −80° C. until used for testing.

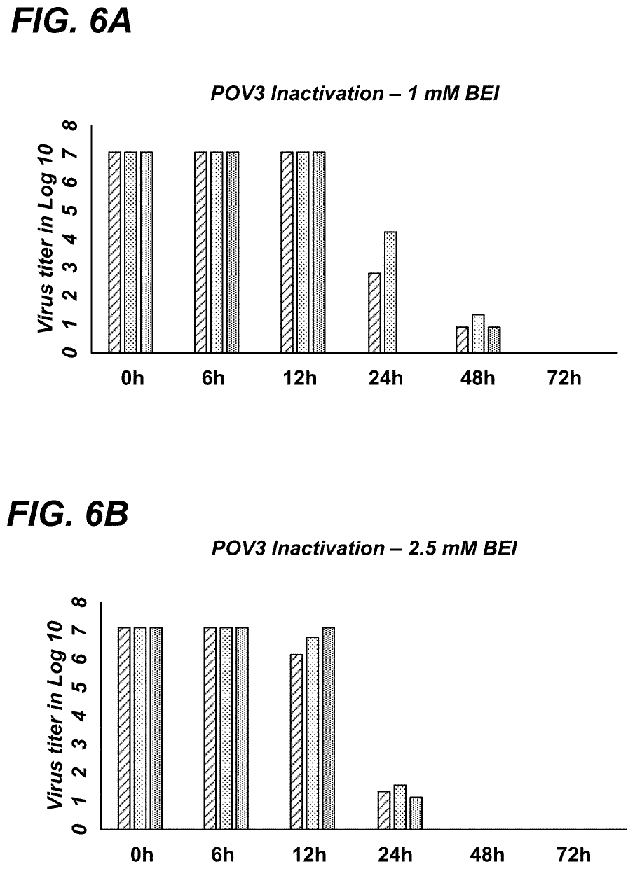

Samples were collected at different time points (0 h, 6 h, 12 h, 24 h, 48 h and 72 h) and neutralized with appropriate volume of 1M sodium thiosulfate and frozen at −80 degree deep freezer. The virus titer in each time point is assayed using TCID₅₀ method at the end of complete inactivation period. The regression curve is plotted to study the inactivation kinetics. From the virus inactivation kinetics study results of which are shown in FIGS. 6A and B, it was determined that 2.5 mM BEI can completely inactivate the POV3-BM100 virus at 37° C. in 48 hours.

Inactivation Validation:

The samples collected during inactivation, the original virus control (held at −80° C.) and the non-treated virus control held at 37° C. for 48 hours are diluted in appropriate diluent (from neat to 10⁻⁸) are titrated in 96 well micro-wells as per standard established technique to determine the TCID₅₀ titers of each samples. Each sample is inoculated in four replicates. The cell cultures are incubated for a prescribed time and titration is read according to CPE or by other established methods such as immunofluorescence or immunoperoxidase staining

### Example 8

### Modified Live-Attenuated Vaccine (MLV)

In one embodiment a modified live-attenuated virus vaccine is generated from the novel virus isolates. The virus has been propagated in Vero cells and BHK-21 cells as well as chicken embryos and serial passaging is underway to generate a modified live-attenuated vaccine (MLV). By serial passage in non-porcine host cells, the virulence of the virus is gradually affected until the virus losses the ability to cause significant morbidity in adult and juvenile pigs.

### Example 9

### Hemagglutination Inhibition Assay for Screening Pig Sera for POV3 Antibodies

The hemagglutination-inhibition (HI) assay is an effective method for assessing immune responses to porcine orthoreovirus hemagglutinin (HA). The HA protein on the surface of swine orthoreovirus/MRV agglutinates erythrocytes. Specific attachment of antibody to the antigenic sites on the HA molecule interferes with the binding between the viral HA and receptors on the erythrocytes. This effect inhibits hemagglutination and is the basis for the HI assay. In general, a standardized quantity of HA antigen (4 HA units) is mixed with serially diluted serum samples and swine red blood cells (sRBCs) are added to detect specific binding of antibody to the HA molecule. The presence of specific anti-HA antibodies will inhibit the agglutination which would otherwise occur between the virus and the RBCs. During adsorption with horse RBCs, non-specific virus inhibitors may be introduced into serum, which will cause a false positive result in HI assay with pig RBC. Such non-specific inhibitors can be eliminated by receptor destroying enzyme (RDE) treatment.

Materials are assembled including: 1) porcine orthoreovirus (POV3)/Mammalian orthoreovirus 3 (MRV3), 2) pig serum samples (serum samples should not be repeatedly freeze-thawed but are ideally aliquoted and stored at −20 to −70° C.), 3) swine RBCs in PBS (Porcine RBCs in Alsever's solution are obtainable from Lampire Biological or equivalent source, and used at a concentration of 1.0% in PBS+0.5% BSA), 4) horse blood cells in Alsever's solution (as fresh as possible), 5) Phosphate buffered saline (PBS) (0.01M PBS, pH 7.2), store at 4° C. and keep on ice during use, 6) Receptor destroying enzyme (RDE), 7) 96-well, V-bottom, polystyrene, microtiter plates (Nunc, cat. #249570).

To determine the HA titer of the test virus, the pig RBC is prepared at 1.0% (v/v). To start preparation of packed RBCs, carefully collect, using a 10 ml pipette, 5-7 ml of pig RBCs from the bottom of the bottle. Remove horse RBCs from the bottom of the container to minimize contamination with cell fragments. Filter through a sterile cotton gauze pad into a 50 ml conical centrifuge tube. Gently fill the conical tube with cold PBS and centrifuge at 800×g for 5 minutes at 4° C. Aspirate the supernatant using a 10 ml pipette, being careful to not disturb the pellet of RBCs. Gently fill the conical tube with cold PBS and mix gently by inversion followed by centrifugation at 800×g for 5 minutes at 4° C. Aspirate the supernatant using a 10 ml pipette, being careful to not disturb the pellet of RBCs. Carefully repeat the cold PBS wash one more time for a total of three PBS washes to prevent hemolysis, always handle the RBCs gently, keep the PBS on ice or at 4° C., and do not wash more than 3 times. Aspirate the remaining supernatant with a P1000 microliter pipette for final packed RBCs and keep the packed RBCs on ice. Prepare a 1.0% v/v suspension of RBCs. For example, add 2.5 ml of the packed, washed to 247.5 ml cold PBS+0.5% BSA in a 500 ml glass bottle (rinse with PBS before use). Mix gently by swirling. For the HA titer determination, mark the V bottom plates with the names of the viruses to be tested. Viruses are tested in duplicate. Add 50 μl of cold PBS to wells 2 through 12 in rows A and B. If more than 1 virus, use the rest of rows as needed. Add 50 μl of cold PBS to the entire H row. This row will serve as the RBC control. Immediately prior to removing virus from vial, gently vortex the vial of virus using three quick pulses. Then add 100 μl of the virus to be tested to wells A1 and B 1. Make serial 2-fold dilutions by transferring 50 μl from well 1 successively through well 12. Discard 50 μl from well 12. Add 50 μl of 1.0% pig RBC suspension to all wells in rows A, B (or other rows if more than 1 virus), and H on the plate. Gently tap the plates to mix. Stack plates and cover with an empty plate and incubate at room temperature for 60 minutes. Read the virus HA titers by tilting the plate at a 45 to 60° angle. The settled RBCs in row H should start “running” and form a teardrop-shape due to gravity. Wait until these RBCs finish “running” and then note the RBC buttons in the virus titrations that “run”. These RBCs do not exhibit hemagglutination. The highest dilution of virus that causes complete hemagglutination is considered the HA titration end-point. The HA titer is the reciprocal of the dilution of virus in the last well with complete hemagglutination. Dilute virus in cold PBS to make a working solution containing 8 HAU/50 μl. Verify that the diluted virus contains 8 HAU per 50 μl by performing a second HA test as described above. The titer of the virus should be 8. If not 8, then adjust the virus concentration by adding virus if <8 HAU or cold PBS if >8 HAU. Store the working dilution of virus on ice and use within the same day.

HI Assay with pig RBCs.

1. Thaw the sera at room temperature and heat inactivate at 56 degree for 30 minutes, then keep on ice during use.

2. Mark the V bottom plates with the plate number and the names of the viruses accordingly based on experiment design.

3. Column 12 of all plates can be reserved for the RBC control. Positive and negative control sera, and back titration can be run in a separate plate or incorporated in available columns of plates.

4. If dilution plates/titer tubes are used, for duplicate test with one virus, make a serial 2-fold dilution of treated sera by adding 110 μl of treated sera (1:10) to titer tubes in rows A, columns 1-11.

5. Add 55 μl of cold PBS to titer tubes in rows B-H, columns 1-11.

6. Transfer 55 μl of sera from row to row (A->B->C . . . H) using a P200 multichannel pipette to make serial 2-fold dilutions.

7. Discard 55 μl from row H after mixing.

8. Positive and negative control with appropriate initial dilution should be serially diluted following the same procedure above.

9. Transfer 25 μl of each diluted serum sample from dilution plate into V-bottom plates starting with row H and going to row A. No need to change tips if transferring from the highest dilution (row H) to the lowest dilution (row A). It is critical that the tips must be changed before beginning to pipet the next set of serum samples.

10. If dilution plate are not available, serial dilution of sera samples can be done directly on plates. For each replicate test with one virus, first, add 25 μl of cold PBS to V-bottom plate in rows B-H, columns 1-11. Second, add 50 μl of heat inactivated sera to row A, columns 1-11. Then, transfer 25 μl RDE-treated sera from row to row (A->B->C . . . H) to make serial 2-fold dilutions. Discard 25 μl from row H after mixing.

11. Add 25 μl of standardized virus containing 4 HAU to wells containing sera. Note this is the same as 50 μl containing 8 HAU.

12. Gently tap the plates to mix. Stack plates and cover with an empty plate.

13. Incubate virus and sera at room temperature (22° to 25° C.) for one hour.

14. Add 50 μl of PBS to column 12. This will serve as the RBC control.

15. Add 50 μl of 1.0% pig RBC suspension to each well.

16. Gently tap the plates to mix. Stack plates and cover with an empty plate.

17. Incubate at room temperature for one hour.

18. Record the HI titers of sera after one hour incubation by tilting the plates at a 45 to 60° angle. The settled RBCs in column 12 should start “pulling” or “running” and form a “teardrop-shape” due to gravity. Wait until these RBC's finish “pulling” and then read the RBC buttons that “run” or “stream” in the same way. A well with complete hemagglutination inhibition will look the same as the RBC controls. The serum HI titer is the reciprocal of the serum dilution in the last well with complete hemagglutination inhibition.

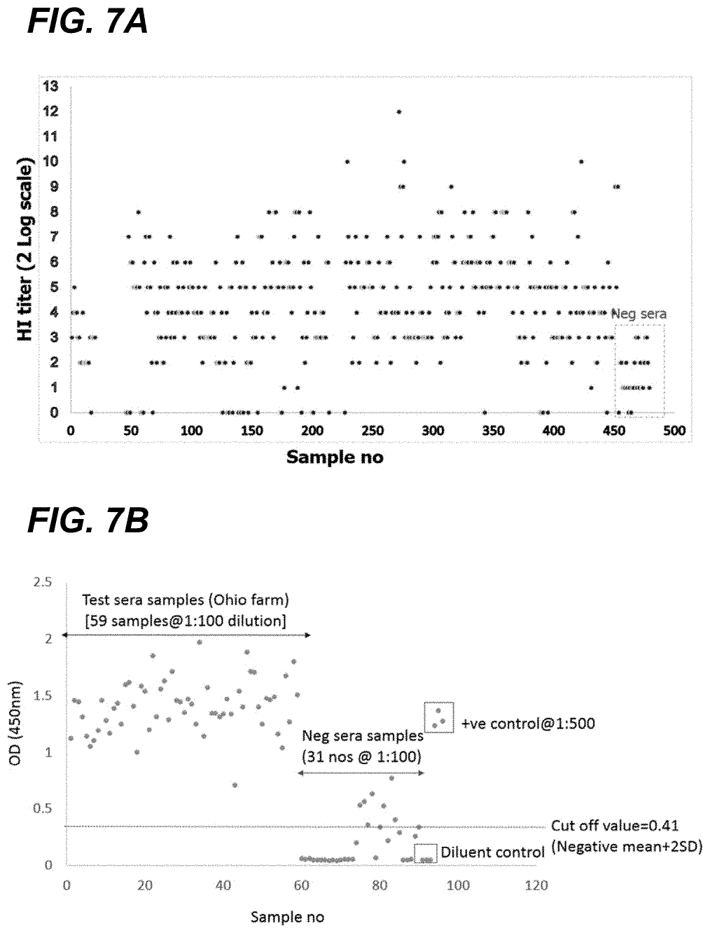

To identify the prevalence and geographic distribution of this novel orthoreovirus, we performed a retrospective serological surveillance of 1067 sera samples collected from 24 states during 2014-2015 and 28 sera samples from 2008 using the above Hemagglutination Inhibition (HI) assay of pig erythrocytes with plaque purified MRV3 as the hemagglutinin. It was determined that the age of the pigs had no significant influence on the HI titers, in animal tested from 1-56 weeks of age. The prevalence of POV3-specific HI antibodies was very high during 2014-2015 but negative for samples from 2008. The HI titers ranged from 2 to 4096 against POV3 with 88.37% of samples above the cut-off titer of 1:8. High HI antibody titers (2048 and above) were recorded only from swine sera samples collected from Iowa, North Carolina Pennsylvania, Texas, South Dakota, Oklahoma, Montana, Michigan, Georgia and Colorado States. The HI titers of 450 samples are plotted in terms of 2 Log scale as depicted on FIG. 7A.

### Example 10

### Screening of Pig Sera Samples for POV3 Specific IgG Using Indirect ELISA

An indirect ELISA protocol was developed for screening swine or any other species sera samples for the presence or absence of POV3 specific IgG using ultra-purified whole virus or recombinant proteins of the POV3 virus for sero-monitoring of POV3 infection. Generally, dilutions of swine sera are added to purified POV3 coated microtiter plates and antibodies specific for POV3 bind to the microtiter plates. The antibodies bound to the plates are detected using labelled anti-swine IgG such as alkaline phosphatase-labeled antibody followed by a p-nitrophenyl phosphate substrate. The optical density of the colored end product is proportional to the amount of POV3 specific antibody present in the serum.

In one example performed, purified POV3 (1 mg/mL) frozen aliquots stored at −80° C. were thawed at room temperature. The viral antigen was diluted to a predetermined concentration (generally 2.5 m/ml) with sterile antigen-coating buffer (1×PBS/0.02% NaN₃). An aliquot of 100 μl of antigen was pipetted into each well of microtiter plate(s) and covered for incubation at 4° C. overnight. The wells were blocked using 300 uL/well Super Block Blocking buffer in PBS (Thermo Scientific, USA, cat no: 37515) for 1 hour at room temperature and the plates were stored in a humidified chamber kept at 4° C. If sodium azide is used, coated plates may be stored for several months at 4° C., provided that storage conditions are suitable to prevent evaporation and contamination of the Blocking solution. Further reagents prepared included Substrate stop solution: 3M NaOH [1 liter], 2M Sulfuric acid/Stop solution [200 ml], and Coating Buffer 10× (10×PBS/0.2% NaN3 [1L]: NaCl—80 g, KH₂PO₄—3.14 g, Na₂HPO₄.7H₂O—20.61 g, KCl—1.6 g, NaN₃—2 g). When diluted the pH of the 1× coating buffer should be should be 7.2±0.2.

Sera dilution Buffer 10×: 10×PBS/0.2% NaN₃/0.5% Tween-20 [1L]: NaCl 80 g, KH₂PO₄ 3.14 g, Na₂HPO₄.7H₂O 20.61 g, KCl 1.60 g, NaN₃ 2 g is prepared. Add 800 ml of reagent grade water to a 2-liter beaker placed on a magnetic stirrer. Weigh out the dry chemicals listed above and add them to the water. Dissolve the chemicals and bring the volume to 1 L with reagent grade water. Add 5 ml Tween-20. When diluted the pH of the 1× sera dilution buffer should be should be 7.2±0.2. The Wash buffer is 1×PBS/0.05% Tween-20, pH 7.2±0.2.

Procedure for testing swine sera with unknown anti-POV3 antibody concentrations. Retrieve all serum samples, controls and reference sera stored frozen and place them at room temperature to thaw (˜30 minutes). Samples should not be freeze/thawed more than 3 times. Perform serial dilutions (usually 2- or 3-fold) of sera as necessary with dilution buffer and incubate the diluted samples at room temperature for 30 minutes. Wash the antigen-coated microtiter plates 5 times with wash buffer. During the first wash, allow the wash buffer to soak on the plate 30 seconds to 1 minute after filling the wells. Using a multichannel pipettor, transfer 50 μl of each serum dilution from the dilution plates to the washed antigen coated plates. Add only antibody buffer to two wells in each plate to serve as blanks. Cover plates with lids and incubate at room temperature for 2 hours. Prepare the appropriate dilution of anti-swine IgG conjugate in antibody buffer 15 minutes before its use. Wash the plates 5 times with wash buffer. During the first wash, allow the wash buffer to soak on the plate 30 seconds to 1 minute after filling the wells. Add 100 μl of diluted enzyme conjugate to all microtiter plate wells. Cover plates with lids and incubate for 1 hour at room temperature. Prepare a 1 mg/ml solution of p-nitrophenyl phosphate in the diethanolamine substrate buffer 15 minutes before it is required. Mix the substrate solution on the shaker while wrapped in a paper towel to protect it from light. Wash the plates 5 times with wash buffer. During the first wash, allow the wash buffer to soak on the plate 30 seconds to 1 minute after filling the wells. Add 100 μl of substrate solution to all microtiter plate wells. Put lids on plates and incubate for 2 hours at room temperature. Add 50 μl of 3M NaOH to all wells to stop the enzyme reaction. Wait at least 5 minutes, before reading the optical density of the plates on a microtiter plate reader at 450 nm. FIG. 7B depicts results obtained for randomly selected 59 unknown pig sera samples from the 2014 outbreak in Ohio, 31 known negative pig sera samples from the year 2008 are represented in the figure.

To demonstrate that the POV3 purified viral antigen produces comparable results and comparable lower limits of detection using true positive swine serum samples, checkerboard titration was performed with different dilutions of the antigen and antibody. Antigen was adsorbed on to the surface of a microtiter plate in increasing concentrations. Reference serum is added at one dilution across the plate and the ELISA is completed using POV3 specific known antibody. The optimal coating concentration of an antigen lot is determined by inspecting optical density values vs. antigen concentration. Eight different dilution of the known positive sera sample (1:1000 to 1:128000) were tested with three different concentrations of the purified POV3 virus viz 1.25 ug/mL, 2.5 ug/mL and 5 ug/mL as described previously. The results obtained were plotted concentrations of antibody (Y-axis) against the OD values on (X-axis). In one tested preparation, the optimal concentration of purified virus for coating was determined to be 2.5 ug/mL. The sensitivity/lower limits of sera dilution for ELISA may be determined by checkerboard titration of known positive and negative sera samples diluted from 1:100 to 1:51200 with 2.5 ug/mL coated purified POV3 and using an antiMRV S1 monoclonal antibody as a positive control.

### Example 11

### Development of RT-PCR Based Assays for Detecting Pathogenic Porcine Orthoreovirus-3 (POV3) from Clinical Samples

To detect POV3 in feces and tissue samples and blood meal samples, a simple RT-PCR was developed targeting the S1 and L1 genes of the pathogenic porcine orthoreovirus. The primers were designed based on the in silico analysis and selection of unique regions that were present on the pathogenic POV3-VT porcine orthoreovirus as characterized by the present inventors. RNA extracted from the specimens was subjected to cDNA synthesis using ABI first strand synthesis kit, employing random primer/reverse primer. RNA was heat denatured at 70° C. for 10 min, snap cooled, mixed with cDNA master mix and incubated at 25° C. for 10 min for binding of primer. RT reaction carried out for 2 hours at 37° C., RT-inactivation at 85° C. for 5 min. cDNA was amplified using PCR using either S1 specific or L1 specific primers as follows:

POV3_VT_S1 Fwd (KM820760):
SEQ ID NO: 3
5′-138 CAC TCT GAT ACA ATC CTT AGG ATC ACT CAA
GG 169-3′,
POV3_VT_S1 Rev (KM820760):
SEQ ID NO: 4
5′-573 CCA TCG TCA TAC GAT TGT TAT TGA TTG
CCA 544-3′,
POV3 L1 Fwd:
SEQ ID NO: 5
5′-1541 CTA TAC TAG CTG ACA CTT CGA TGG GAT
TGC 1570-3′,
POV3 L1 Rev:
SEQ ID NO: 6
5′-3129 CGT CTC ATC CAT TTC TGC CAG CTC
TT 3104-3′,

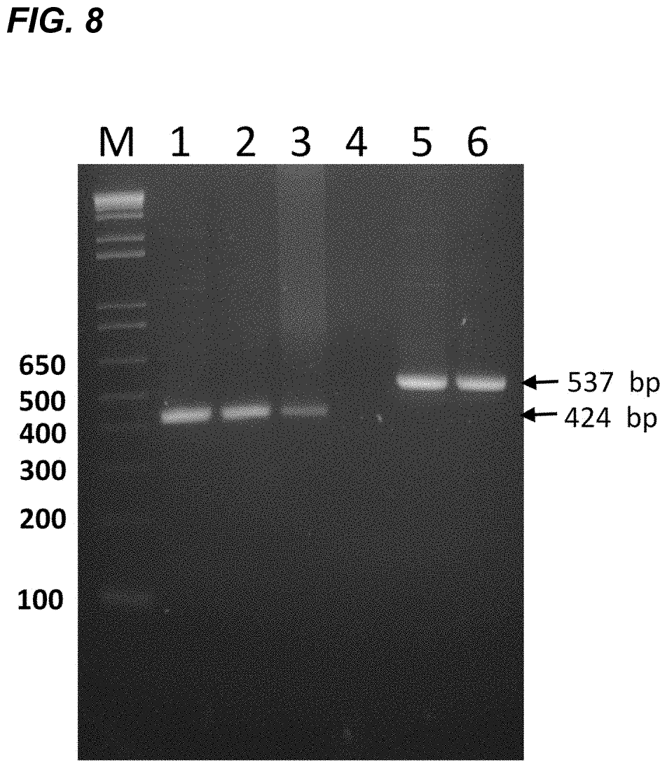

Initial denaturation at 94° C. for 5 min; 40 cycles consisting of denaturation at 94° C. 30 sec; primer annealing at 58° C. for 30 sec and extension at 72° C. for 30 sec. Final extension at 72° C. for 10 minutes. The amplified length was 424 bp and 537 bp for S1 and L1 gene fragments respectively as seen in FIG. 8. (Agarose gel electrophoresis of RT-PCR amplified products targeting POV3 S1 and L1 genes: M: 1 Kb+ ladder, Lane 1-2: POV3—Fecal sample (S1 target), Lane 3: POV3—Blood meal (S1 target), Lane 4: No template negative control, Lane 5: POV3—Fecal sample (L1 target), Lane 6: POV3—Blood meal (L1 target).

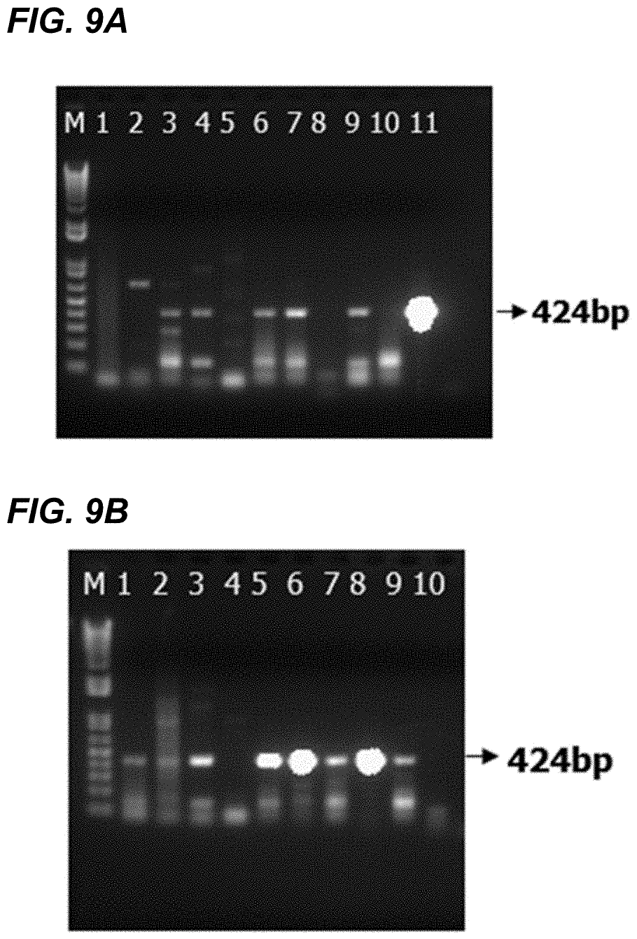

RT-PCR screening of POV3 was conducted in brain and lung tissues of experimentally infected piglets. To detect POV3 in tissue samples, lung and brain samples were selected from experimentally infected piglets. The RNeasy Mini Kit (Qiagen, USA) was used to extract RNA from Fresh, frozen, or RNA later stabilized tissue (up to 30 mg, depending on the tissue type) as per the manufacturer recommendation. RNA was subjected to cDNA synthesis using ABI first strand synthesis kit, employing random primer/reverse primer. RNA heat denatured at 70° C. for 10 min, snap cooled, mixed with cDNA master mix and incubated at 25° C. for 10 min for binding of primer. RT reaction carried out for 2 hours at 37° C., RT-inactivation at 85° C. for 5 min. cDNA was amplified using PCR using S1 specific forward and reverse primers with initial denaturation at 94° C. for 5 min; 40 cycles consisting of denaturation at 94° C. 30 sec; primer annealing at 58° C. for 30 sec and extension at 72° C. for 30 sec. Final extension at 72° C. for 10 minutes. The amplified length was 424 bp. RT-PCR followed here successfully amplified the partial S1 gene fragment of 424 bp in both tissue types as seen in FIGS. 9A and B. In the Figures, agarose gel electrophoresis of RT-PCR amplified products from tissue homogenates targeting POV3 S1 genes are shown. FIG. 9A: S1 segment based RT-PCR on brain tissue homogenates of experimentally infected piglets: Lane M: 1 Kb+ ladder, Lane 1-9: RT-PCR on brain homogenates of experimentally infected piglets, Lane 10—RT-PCR on mock infected brain homogenate, Lane 11: POV3 virus positive control. FIG. 9B: S1 segment based RT-PCR on lung tissue homogenates of experimentally infected piglets: Lane M: 1 Kb+ ladder, Lane 1-9: RT-PCR on brain homogenates of experimentally infected piglets, Lane 10-RT-PCR on mock infected brain homogenate.

### Example 12

### SYBR Green Based Quantitative Real Time PCR Assay for Detection of Novel Porcine POV3

A further example of a method for detecting the presence or absence of POV3 in a swine biological sample is provided. As POV3 viruses are segmented RNA viruses, the method comprises a reverse transcription step and cDNA amplification cycles using either POV3 S1 or L1 gene specific primers to produce an amplification product if a POV3 nucleic acid molecule is present in the sample. As a result of the methods described herein, the amplification and subsequent detection of the target nucleic acids is possible. A real-time PCR assay was run with the following primer combinations, using POV3 RNA as template. Primer combination S1: POV3_VT_S1 Fwd, SEQ ID NO: 3, and POV3_VT_S1 Rev, SEQ ID NO: 4. Primer combination L1: POV3 L1 fwd, SEQ ID NO: 5 and: POV3 L1 rev, SEQ ID NO: 6.

The PCR reaction was set-up according to the parameters below. Two sets of reactions were performed. A Biorad i cycler machine was used to perform the following cycling conditions—55° C. for 5 mins, 60° C. for 5 mins and 65° C. for 5 mins. This is followed by 45 cycles of: 94° C. for 5 s and 60° C. for 40 s. Each reaction was performed in duplicate. The test with POV3 signal will be considered positive if the CT value is below 40.

In a real time PCR assay a positive reaction is detected by accumulation of a fluorescent signal. The CT value (cycle threshold) is defined as the number of cycles required for the fluorescent signal to cross a threshold that exceeds background. CT levels are inversely proportional to the amount of target nucleic acid in the sample with the lower the CT level the greater the amount of target nucleic acid in the sample.

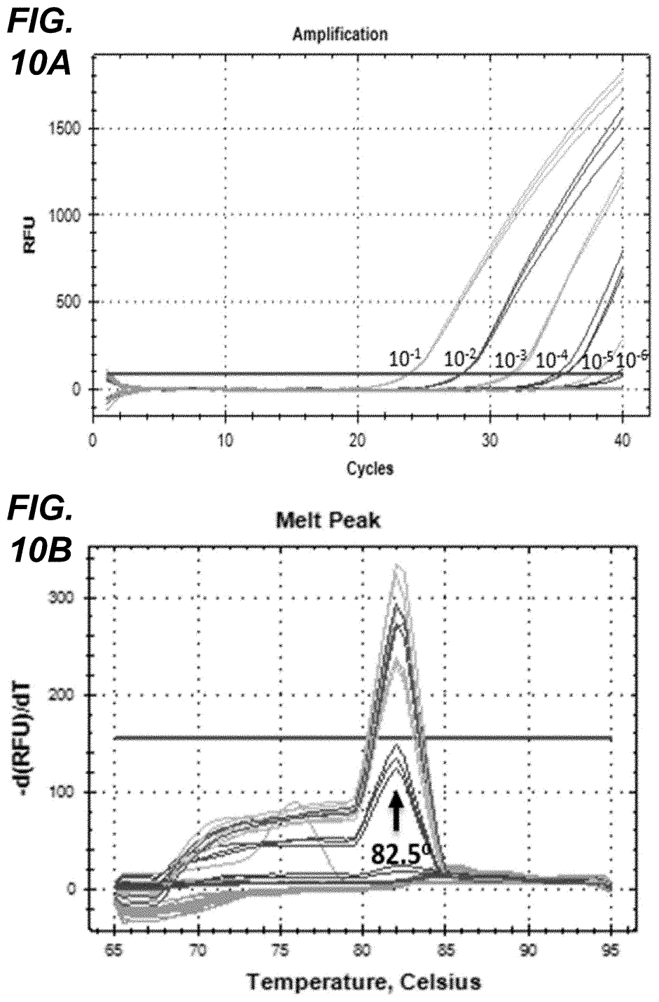

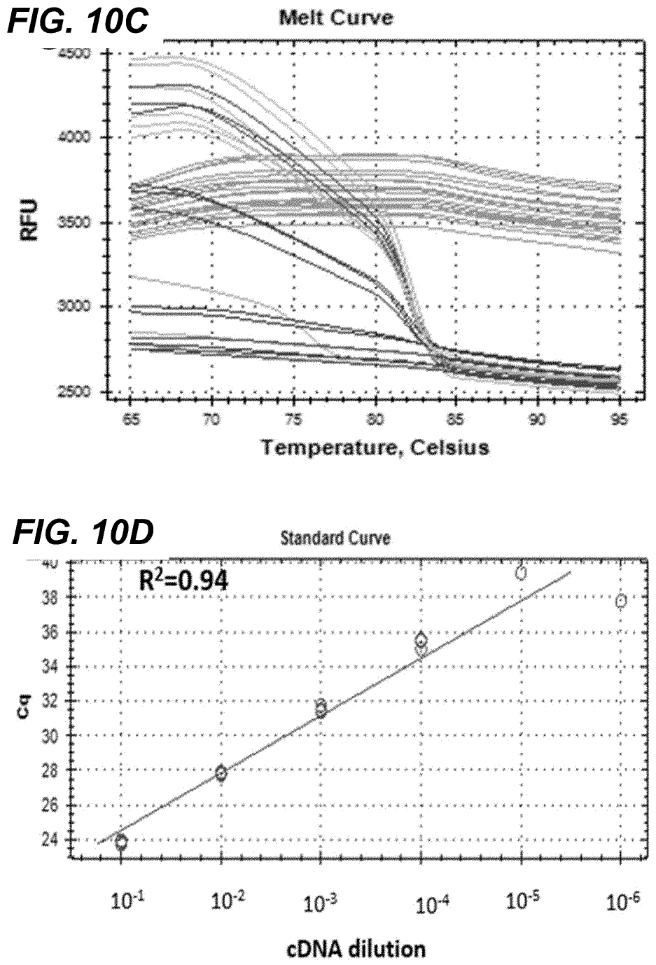

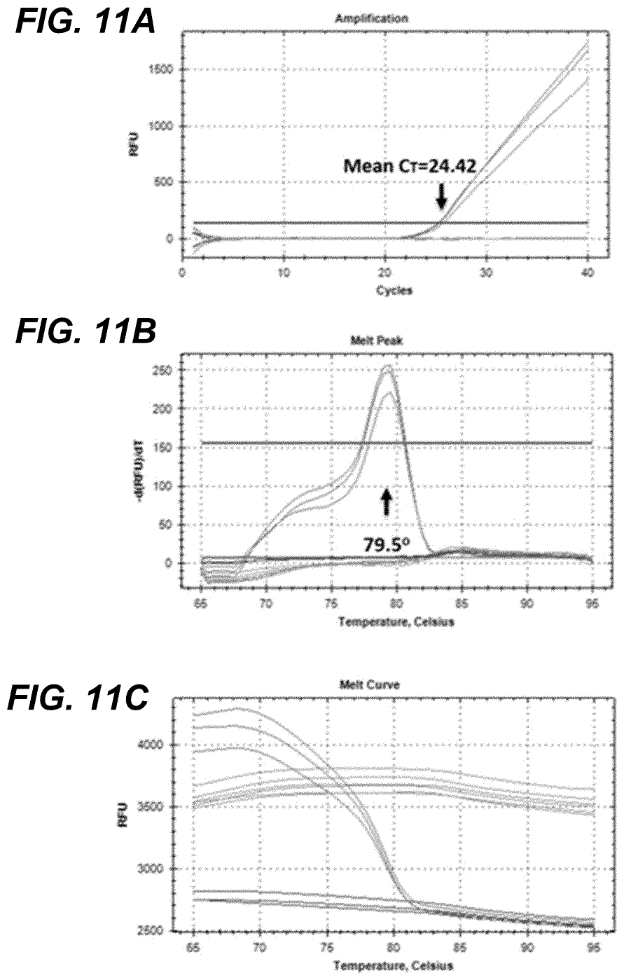

The assay is suitable to diagnose both POV3 S1 and L1 segments. As shown in FIG. 10A, different dilutions of cDNA derived from the cell culture amplified POV3 were used to check the linearity. As seen in FIG. 10B, upon melt curve analysis, all the amplified PCR products amplified from S1 specific primers had the same melt curve that peaked at 82.5° C. In contrast, the melt peak of L1 amplified PCR products was at 79.5° C. (FIG. 11). The use of double targets in qRT-PCR (S1 and L1) allows for the discriminate diagnosis of the presence of POV3 from cell cultured derived virus, fecal samples, blood meal, infected tissue homogenate.

FIG. 10A: Amplification plots of cDNA dilutions (10⁻¹ to 10⁻⁶) of the cell culture derived POV3; FIG. 10B: Melt curve analysis of S1 amplified PCR products showing melt peak at 82.5° C.; FIG. 10C: Dissociation curve of S1 amplified PCR products. FIG. 10D: Linearity curve of ct values Vs cDNA dilutions.

FIGS. 11A-C show L1 based qRT-PCR amplification of POV3. FIG. 11A: Amplification plots of L1 gene fragment products from the cell culture derived POV3; FIG. 11B: Melt curve analysis of L1 amplified PCR products showing melt peak at 79.5° C.; FIG. 11C: Dissociation curve of L1 amplified PCR products.

### Example 13

### Protective Efficacy of an Inactivated MRV3 Vaccine

An initial objective of the present study was to determine the protective efficacy of an inactivated MRV3 vaccine against MRV3 infection in piglets born to vaccinated sows. The unexpected results from the vaccine study showed that the piglets born to unvaccinated sows did not develop severe disease at all after challenge with MRV3, which led us to further evaluate the pathogenicity of the MRV3 vaccine using gnotobiotic pigs, which are more sensitive for pathogenicity studies.

MRV3 viruses. The MRV3 isolates, F503 and BM100, used in the study were isolated in 2015 from the feces and blood meal of pigs, respectively (Narayanappa et al., 2015, supra). The virus inoculum used for animal infection in this present study was the third passage of the plaque-purified MRV3 F503 isolate. The inactivated MRV3 vaccine was prepared from the fourth passage of the plaque-purified MRV3 BM100 isolate.

Infectivity Titration of MRV3.

MRV3 infectivity titration was performed on confluent cell monolayers of Vero cells grown in 96-well plates (CoStar™, Corning®). The virus stock was serially diluted 10-fold with medium, and 100 μL of each dilution was inoculated onto each of 5 wells of Vero cells. The cell culture plates were incubated at 37° C. with 5% CO₂ for 1 hr, and subsequently 100 μL medium was added to each well. Plates were continuously incubated at 37° C. with 5% CO₂ for 5 days, after which the wells were evaluated for the presence of cytopathic effect (CPE) induced by MRV3 infection. The 50% endpoint was calculated as TCID₅₀/ml using the Reed-Muench method.

MRV3-Specific ELISA.

MRV3 σ1 recombinant protein expressed with the E. coli expression system was purified and used as the coating antigen in the MRV3-specific ELISA. Following titration and optimal dilution of the purified recombinant MRV3 σ1 antigen, polystyrene 96-well microtitration plates (Nunc, Thermo Fisher Scientific) were coated (100 μL/well) with the purified antigen and incubated at 4° C. overnight. After washing 3 times, and the plate was first blocked with 300 μL per well of a solution containing 1% bovine serum albumin, followed by incubation with serially-diluted serum samples. The bound antibodies were detected by goat-anti-pig secondary antibody-HRP conjugates (MP Biomedicals, Inc).

Reverse Transcription PCR (RT-PCR) Amplification of MRV3 S1 Fragment.

Total RNAs from fecal or serum samples were isolated using TRIzol® LS Reagent (Invitrogen) according to the manufacturer's instruction. A one-step RT-PCR was carried out to amplify the MRV3 S1 fragment in a 200 μL PCR tube using SuperScript™ III One-Step RT-PCR System (Invitrogen, CA). The primer set includes:

SEQ ID NO: 50
FS03S1:366F22 (5′ GGATTACGCAATGACTACAGCA 3′)
SEQ ID NO: 51
FS0351:959R21 (5′ CCTATCCACATACTTCGCCTA 3′)

Briefly, 5 μL of the extracted RNA and 0.5 μL of MRV3 S1-specific primers were mixed with 2× reaction mix, SSIII/Taq enzymes mix in a 25 μL reaction. The thermal cycling conditions included a reverse transcription at 55° C. for 15 min; initial denaturation at 94° C. for 2 min, 40 cycles of denaturation at 94° C. for 15 s, annealing at 55.4° C. for 30 s, extension at 68° C. for 30 s, and one final extension at 68° C. for 5 min. The amplified RT-PCR products were examined by agarose electrophoresis or subject to a second round nested PCR amplification. For the nested PCR, 5 μL of the first-round RT-PCR product was used as the template for the second round nested PCR in 50 μL reaction using GoTaq® Green Master Mix (Promega, WI). The primer set of the second round nested PCR was:

SEQ ID NO: 52
FS03S1:418U23 (5′ GCGACACTGGATCATTAACGACT 3′)
SEQ ID NO: 53
FS0351:924L22 (5′ GGCTCATCCCAATACTACCACT 3′)

Quantification of Porcine MRV3 RNA by Quantitative RT-PCR (RT-qPCR).

Viral RNAs were quantified in pig fecal samples by RT-qPCR using MRV3-specific primers and probe targeting the MRV3 S1 segment. Briefly, the fecal samples from pigs were suspended in sterile PBS at 10% (w/v). The fecal suspensions were centrifuged at 8000×g at 4° C. for 15 min, and the supernatants were transferred to fresh tubes for RNA extraction. Total RNAs were extracted from 250 μL of 10% fecal suspensions or diluted serum samples with TRIzol® LS Reagent (Invitrogen).

MRV3 RNAs were quantified using the SensiFAST™ Probe No-ROX One-Step kit (BIOLINE USA Inc. USA) with the forward primer (FS03S1:306F22 5′ CTTGATTCGAGTGTTACCCAGT 3′, SEQ ID NO: 54), reverse primer (FS03S1:414R21 5′ TAATGATCCAGTGTCGCGTTC 3′, SEQ ID NO: 55), and a hybridization probe (FS03S1:345L23 5′ CCTGCAAATCCTGTCTCAAGCTG 3′, SEQ ID NO: 56), which contains a 5′ 6-carboxy fluorescein fluorophore and 3′ black hole quencher (BHQ) by following a protocol described previously. See Jothikumar, N., et al., A broadly reactive one-step real-time RT-PCR assay for rapid and sensitive detection of hepatitis E virus. Journal of Virological Methods 131 (2006) 65-71. The RT-qPCR assay was performed in a CFX96 real-time PCR system (Bio-Rad Laboratories). In vitro transcribed and purified MRV3 S1 segment RNAs were used to produce a standard curve in RT-qPCR assay. The thermal cycling conditions of the RT-qPCR assay are as follows: 45° C. for 10 min (reverse transcription); 95° C. for 2 min (initial denaturation); and 95° C. for 5 s followed by 55° C. for 20 s (PCR amplification) for 40 cycles. The detection limit of the RT-qPCR assay is 10 viral genomic copies as previously reported (Jothikumar et al., supra).

Preparation of an Inactivated MRV3 Vaccine.

The MRV3 BM100 virus, which was isolated from blood meals of pigs, was used as the seed virus for vaccine preparation. Briefly, the BM100 virus was propagated in BHK-21 cells, and the infected cells were frozen and thawed 3 times to release the intracellular virions. The cell debris was removed by centrifugation at 4000×g for 20 min at 4° C. The infectious titer of the virus in the supernatant was determined using the TCID₅₀ method in 96-well plates. Subsequently, the MRV3 BM100 virus stock was inactivated by binary ethyleneimine (BEI) at 37° C. To determine the inactivation kinetics of MRV3, serial samples (0.5 mL) with different inactivation time points were collected at 6, 12, 24, 48 and 72 h post-inactivation (hpi). BEI was neutralized with 10% 1M sodium thiosulfate (STS) to a final concentration of 2%. The tissue culture supernatant of serial samples was serially diluted 10-fold and inoculated onto Vero cells in 96-well culture plates to determine the kinetics of BEI inactivation of MRV3. The time point of the sample that showed no obvious CPE after three blind passages was set as the cut-off for the MRV3 inactivation point. To prepare the inactivated vaccine for use in this study, aluminum hydroxide gel (Alhydrogel® adjuvant 2%) was mixed with the inactivated MRV3 vaccine (2×10⁷ TCID₅₀ per ml) at 1:1 ratio according to manufacturer's instruction.

Vaccination of Pregnant Sows with the Inactivated MRV3 Vaccine and Challenge of the Newborn Piglets with MRV3.

This animal study was approved by the Virginia Tech Institutional Animal Care and Use Committee (IACUC No. 15-032). Briefly, six clinically healthy pregnant sows were acquired from a commercial sow farm at 56 days of gestation. To verify the absence of MRV3 infection in the pregnant sows, serum samples from each sow were collected and tested for MRV3 antibody by a MRV3-specific ELISA and for MRV3 viral RNA by a MRV3-specific nested RT-PCR. Additionally, the absence of other common infections in sows, such as porcine reproductive and respiratory syndrome virus (PRRSV), swine influenza virus (SIV), and porcine epidemic diarrhea virus (PEDV), was verified by pathogen-specific RT-qPCRs or ELISAs.

The 6 MRV3-negative pregnant sows were housed and farrowed at the Virginia Tech BSL-2 Swine Research Facility. Sows and their litters were allocated to 6 different treatment groups (Table 5): sows of groups 1 and 2 were vaccinated with PBS buffer as non-vaccinated controls; sows of groups 3 and 4 were vaccinated with two doses of the inactivated MRV3 vaccine; sows of group 5 and 6 were vaccinated with three doses of the inactivated MRV3 vaccine.

TABLE 5
Experimental design for vaccination of pregnant sows with an
inactivated MRV3 vaccine and subsequent challenge of
offspring conventional piglets with the MRV3 virus.

| | No of | | No. of pigs | |
|---|---|---|---|---|
| | pregnant | Vaccination | born to the | Challenge |
| Group | sow | with | corresponding sow | with |
| 1 | 1 | PBS buffer | 12 | MRV3 FS03 |
| 2 | 1 | | 10 | PBS |
| 3 | 1 | 2 doses of MRV3 | 11 | MRV3 FS03 |
| 4 | 1 | vaccine | 10 | PBS |
| 5 | 1 | 3 doses of MRV3 | 12 | MRV3 FS03 |
| 6 | 1 | vaccine | 6 | PBS |

After farrowing, at 4 days of age, the piglets of groups 1, 3, and 5 were each orally challenged with the wildtype MRV3 FS03 virus (10⁶ TCID50), whereas the piglets of groups 2, 4, and 6 were orally inoculated with PBS buffer as controls. Piglets in group 1 provided a baseline response to the MRV3 infection in the absence of vaccination. Piglets from groups 3 and 5 provided a measure of the effect of vaccine-induced maternal immunity against MRV3 infection in newborn piglets. All piglets were monitored daily for diarrhea, rectal body temperature, dehydration, and ability to stand, walk, and suckle. The sows were also monitored daily for diarrhea, milking ability, anorexia, and alertness. The piglets were necropsied at 4-days post-challenge (dpc), and at necropsy, the gross and microscopic lesions in the duodenum, jejunum, ileum, cecum, colon and lymph node were examined and scored by a board-certified veterinary pathologist (TL). All piglets were closely monitored for clinical signs of disease. Body weight and temperature of all piglets were recorded daily. Serum samples were collected from sows prior to farrowing weekly, and from piglets at dpc 0 and at the end of the experiment. Serum samples were tested for anti-MRV3 IgG antibody by an ELISA. Fecal samples were tested for MRV3 viral RNA by MRV3 S1-specific RT-qPCR. Several parameters including fecal viral shedding, body temperature, weight gain, and mortality rate were used to analyze the effect of vaccine-induced protection against MRV3 infection.

Evaluation of the Pathogenicity of the MRV3 FS03 Virus in Gnotobiotic Piglets.

Near-term cross-breed Yorkshire pigs were delivered via hysterectomy and maintained in sterile isolator units. Neonatal gnotobiotic piglets (male and female) were randomly assigned to the two treatment groups upon derivation: MRV3 infection group (n=9) and control group (n=7). At 3 days of age, all piglets in the MRV3 infection group were each orally inoculated with 3 ml of the MRV3 FS03 virus stock (5×10⁵ TCID₅₀/ml), whereas the piglets in the control group were each orally inoculated with 3 ml of PBS buffer. Fecal consistency and virus shedding were assessed daily until 7 dpc. The intestinal contents, samples of duodenum, jejunum, ileum, cecum, colon, lymph nodes, liver, spleen, and sera were collected at necropsy at 8 dpc. Fecal virus shedding was measured by a one-step TaqMan RT-qPCR, and MRV3-specific antibody was detected by ELISA as described above.

Statistical Analysis.

Using the GraphPad Prism 6.01 software (GraphPad Software Inc.), the differences between the mean values of two treatment groups were analyzed by two-tailed unpaired student's t-test or two-way analysis of variance (ANOVA) followed by Tukey multiple comparisons test.

Humoral Immune Response of Pregnant Sows Vaccinated with the Inactivated MRV3 Vaccine:

In order to detect the MRV3-specific antibody response in pigs, we first cloned and expressed the His-tagged recombinant MRV-3 σ1 protein (455 amino acid) in the E. coli expression system. The expected 49 KDa σ1 protein was successfully expressed along with a smaller protein (S.1s) (FIG. 12A-FIG. 12C), which was produced by leaky scanning of the 51 mRNA. The 49 kDa recombinant protein was purified by His-tag affinity chromatography and stored at −80° C. until use as the coating antigen for the MRV3-specific ELISA.

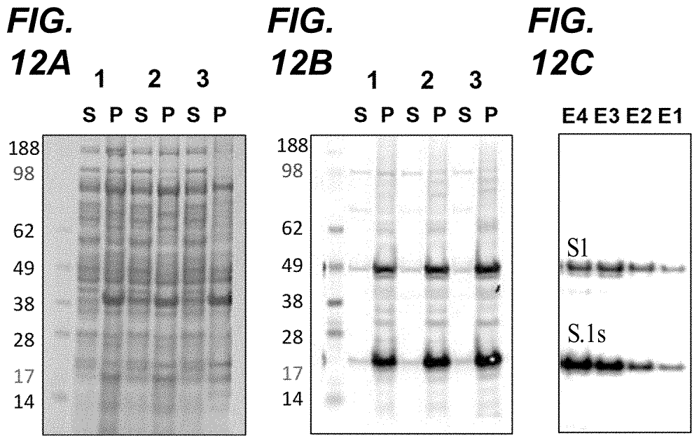

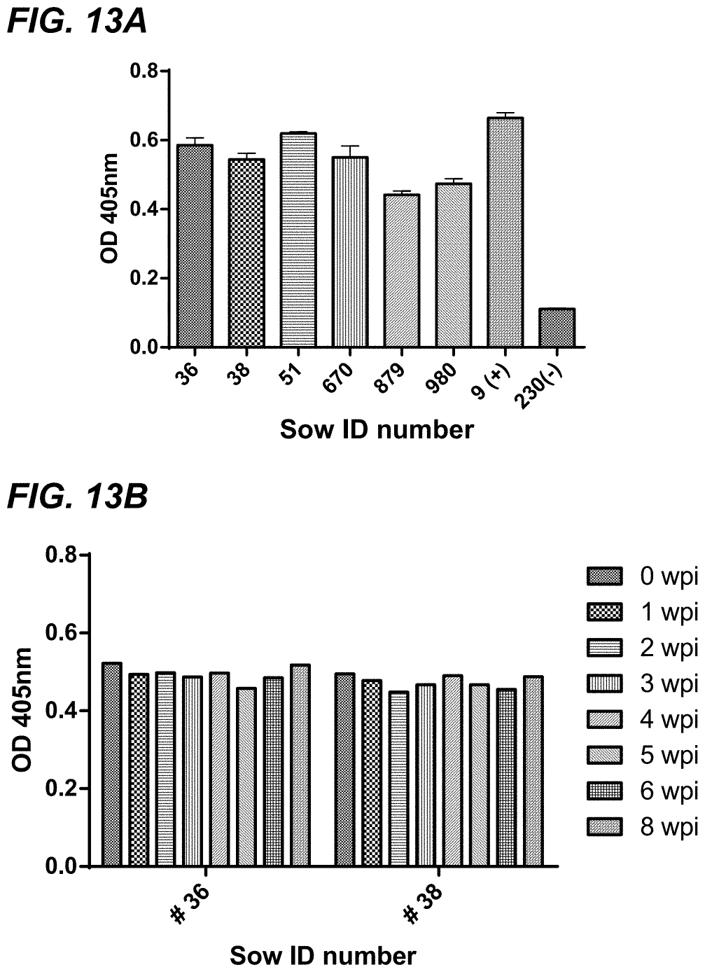

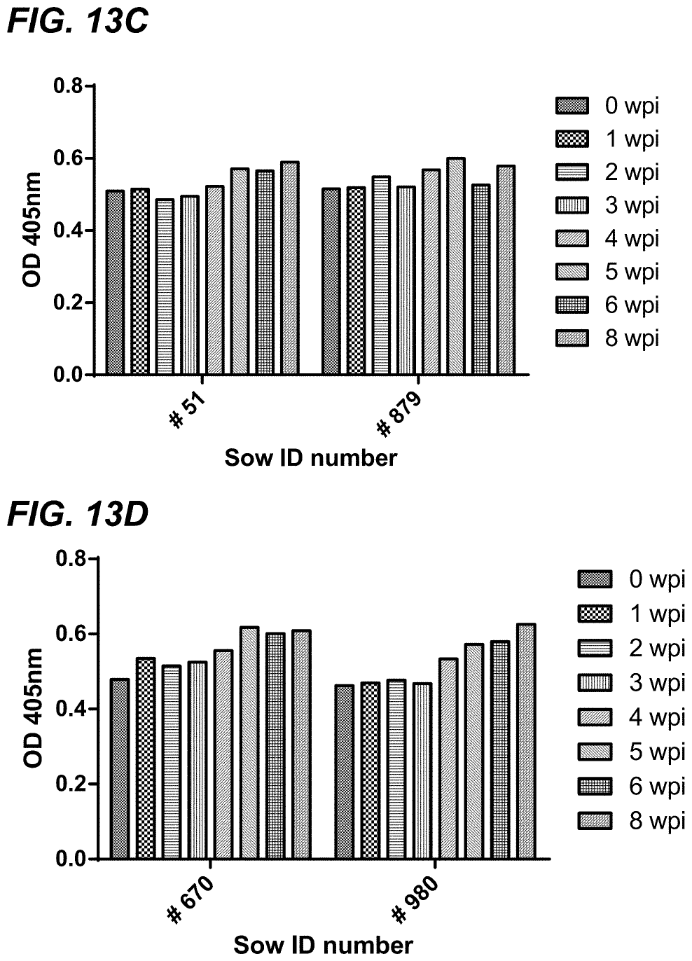

By using the MRV3-specific ELISA, we screened 3 batches of pregnant sows from different sources, and all sows showed a low level of MRV3 antibody titer (FIG. 13A), which is likely due to the prevalence of the MRV3 infection in the field. Based on the serology results, we selected 6 pregnant sows that had the lowest titer of MRV3 antibodies for the vaccination and challenge study. The pregnant sows exhibited normal maternal behavior with no clinical sign of any disease, and after farrowing the litters were kept with their dam throughout the study. Fecal samples collected from all sows upon arrival were tested negative by RT-PCR assays for MRV3, PEDV, PRRSV, and SIV.

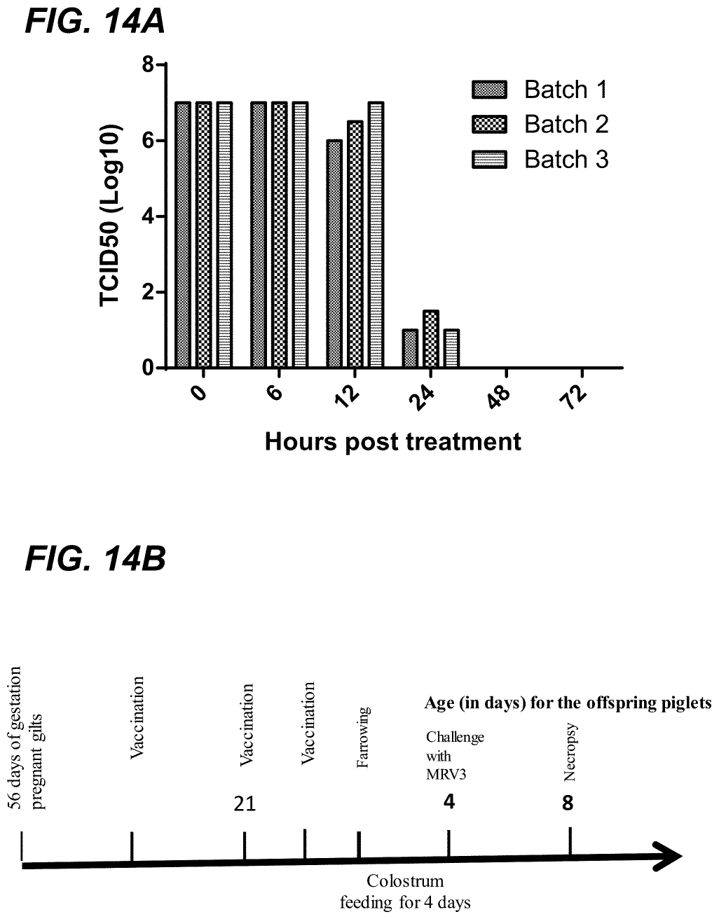

The BEI inactivation kinetics of MRV3 BM100 virus showed that treatment of the virus with 2.5 mM BEI at 37° C. for 48 hr completely inactivated the virus (FIG. 14A). Therefore, the inactivated MRV3 vaccine was prepared by treating the MRV3 BM100 virus stock with 2.5 mM BEI at 37° C. for 48 hr.

For the protective efficacy study of the inactivated MRV3 vaccine, the pregnant sows were randomly assigned to 3 groups and vaccinated over periods of time as shown in FIG. 14B, followed by farrowing and challenge of the piglets with MRV3. Group 1 (sow #670 and #980) was vaccinated with the inactivated MRV3 vaccine at 69, 90 and 100 days of gestation. Group 2 (sow #51 and #879) was vaccinated with the inactivated MRV3 vaccine at 90 and 100 days of gestation. Group 3 (sow #36 and #38) was vaccinated with PBS as controls. Serum samples were collected weekly post-vaccination and tested for the MRV3-specific antibody. The results showed that the MRV3-specific antibody level increased very slowly in sows vaccinated with the inactivated MRV3 vaccine (FIG. 13C and FIG. 13D), and that the sows which received 3 doses of the vaccine elicited a noticeable increase of MRV3-specific antibody, although it was not statistically significant over.

Effect of Sow Vaccination with the Inactivated MRV3 Vaccine on Experimental Infection of the Offspring Piglets with MRV3.

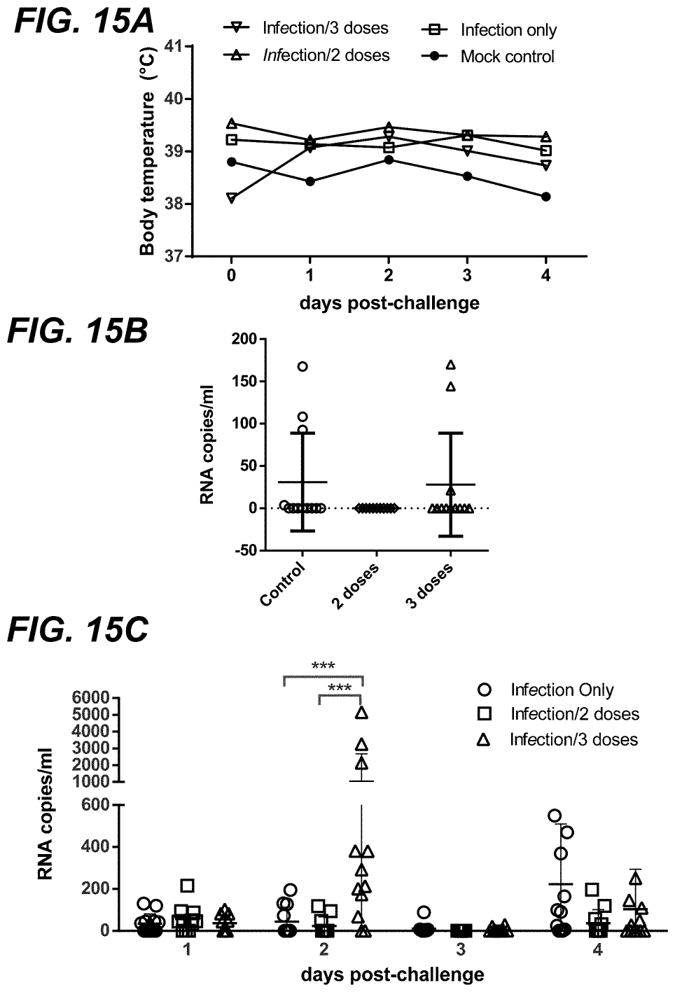

To determine if protective immunity is conferred to piglets born to sows vaccinated with the inactivated MRV3 vaccine, all piglets born to one sow in each of the three groups were challenged with the MRV3 F503 virus at 3 days after birth. Piglets from the other sow in each of the three groups were challenged with PBS buffer as control. There was no significant difference of gross and microscopic lesions in the duodenum, jejunum, ileum, cecum, colon, and lymph node among pigs from infected and control groups, although the rectal temperatures of pigs in the MRV3 FS03-infected groups are slightly higher than pigs from the PBS control group (P>0.05) (FIG. 15A). The lack of severe disease in infected conventional piglets born to unvaccinated sow in this study was surprising, since this contradicted the results of a previous study which MRV3 reportedly induced severe disease in newborn conventional piglets (Narayanappa et al., 2015, supra).

The presence of fecal viral RNA in infected piglets was detected by an MRV3 S1-specific nested RT-PCR. The results showed that, at 4 dpc, the numbers of fecal viral RNA-positive piglets born to vaccinated sows are lower than those from control: 5 out of 12 challenged control piglets from unvaccinated sow were positive compared to only 1 or 2 positive piglets in challenged piglets from vaccinated sow (Table 6).

TABLE 6
Fecal virus shedding detected by nested RT-PCR in conventional
piglets born to vaccinated and non-vaccinated sows at different
days after challenge with MRV3 virus

| | Piglets born to | Piglets born to sows | Piglets born to sows |
|---|---|---|---|
| Days | unvaccinated | receiving | receiving 3 |
| post- | sow (no. | 2 vaccine doses | vaccine doses |
| challenge | positive/ | (no. positive/ | (no. positive/ |
| (dpc) | no. tested) | no. tested) | no. tested) |
| 1 | 3/12 | 1/11 | 4/12 |
| 2 | 2/12 | 5/11 | 5/12 |
| 3 | 6/12 | 4/11 | 3/12 |
| 4 | 5/12 | 1/11 | 2/12 |

A RT-qPCR was used to quantify the amount of viral RNA in small intestine contents collected during the necropsy at 4 dpc. The results showed a similarly low level of MRV3 RNA loads in small intestinal contents with no statistical difference among different vaccination groups (FIG. 15B). Surprisingly, at 2 dpc, the amount of fecal viral RNA in the piglets derived from vaccinated sows are higher than those from control, although there was no difference at 1, 3 and 4 dpc (FIG. 15C).

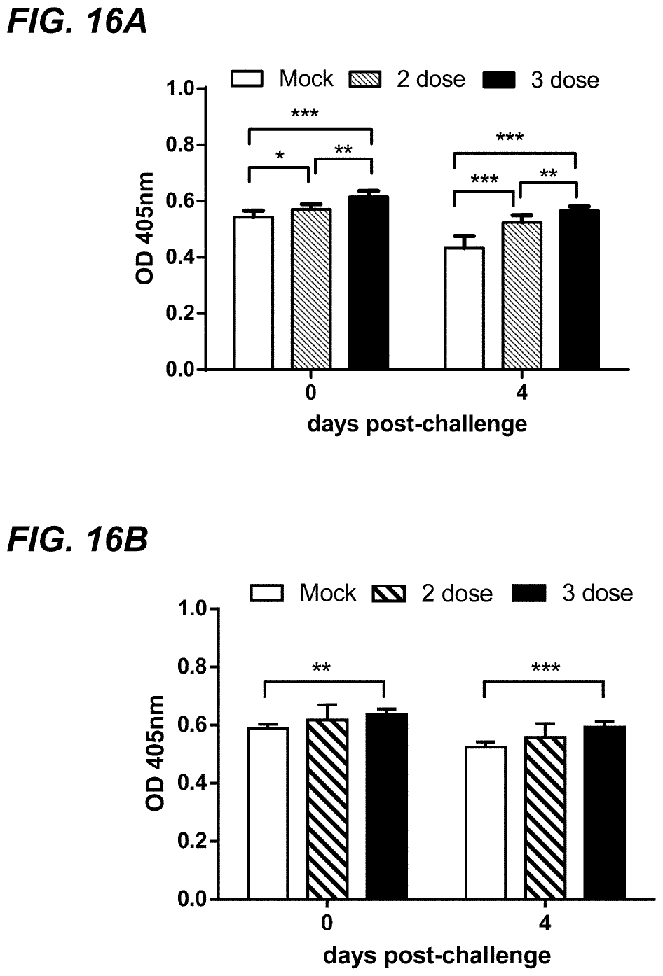

Among the MRV3-challenged groups, piglets derived from sows vaccinated with 2 or 3 doses of the inactivated MRV3 vaccine had significantly higher antibody levels than the piglets derived from non-vaccinated sows (FIG. 16A). A difference in the level of the MRV3 antibody in piglets derived from vaccinated and non-vaccinated sows were observed among the non-challenge control groups (p<0.01 at 0 dpc, p<0.001 at 4 dpc) (FIG. 16B).

MRV3 FS03 Isolate is Only Mildly Pathogenic in Gnotobiotic Piglets.

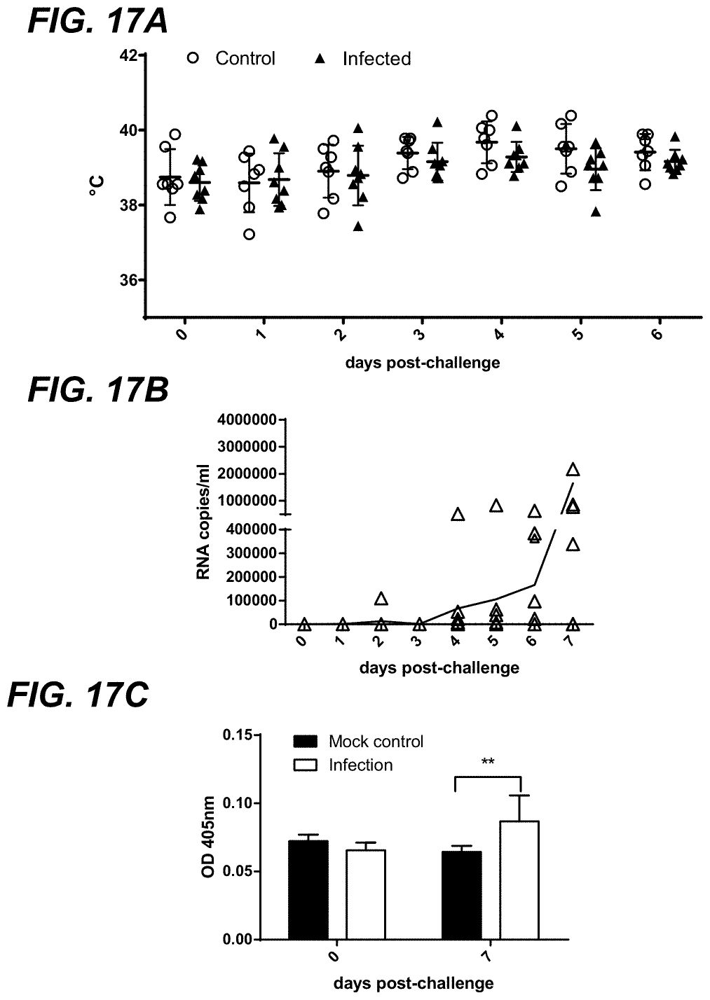

All gnotobiotic piglets were clinically normal in appearance and behavior prior to infection with MRV3 F503 virus. At the early stage of infection, MRV3 F503 virus did not cause diarrhea in piglets at all, although at 7 dpc there were 2 pigs with severe diarrhea and 4 pigs with mild diarrhea or soft feces. There was no difference in rectal temperature between infected and non-infected gnotobiotic piglets (FIG. 17A). The fecal viral RNA load as well as the number of viral RNA-positive piglets were low during the first 4 days of infection (FIG. 17B). However, at 7 dpc, 6 out of the 8 gnotobiotic piglets had detectable fecal MRV3 RNA at a much higher amount (FIG. 17B). Although the level of MRV3-specific antibody is much lower in the infected gnotobiotic piglets compared to infected conventional piglets, the MRV3 infection did elicit a detectable level of MRV3 antibody at 7 days post-infection (p<0.01) (FIG. 17C). There was no gross intestinal lesion at necropsy, and histopathological exam revealed no significant difference in microscopic intestinal lesions between infected and non-infected piglets. There was no difference in the growth rate between infected and non-infected piglets either (data not shown).

Neonatal pigs have an immature immune system, are agammaglobulinemic and lack effector and memory T-lymphocytes, and thus are highly susceptible to infections with various pathogens especially enteric viruses. Neonatal piglets typically acquire immunological protection against enteric viral infections through the ingestion of colostrum and milk. Therefore, it is critical to elicit strong immune responses against infection in sows so that maternal immunity can be transferred to neonatal piglets for protection against enteric virus infections. The present inventors identified and isolated a novel MRV3 from pigs in the United States, and surprisingly reported the virus to be highly pathogenic as neonatal piglets experimentally infected with the MRV3 had severe diarrhea and acute gastroenteritis with high mortality (Narayanappa et al., 2015, supra). In this present study, the inventors first aimed to determine whether vaccination of sows with an inactivated MRV3 vaccine could reduce MRV3 infection of the offspring piglets.

The pregnant sows vaccinated with the inactivated MRV3 vaccine had a slightly increase of the MRV3-specific antibody level, especially those that were vaccinated with 3 doses of the inactivated vaccine. This suggested that the inactivated vaccine used in this study could elicit an MRV3-specific immune response after booster doses in pregnant sows, although the vaccine-induced antibody response is unexpectedly low in vaccinated sows. It suggests that a higher dose of vaccine or an improved adjuvant will likely be needed in the future to induce a stronger humoral immune response in pregnant sows.

After farrowing, the offspring piglets were challenged with MRV3 virus as detailed in FIG. 14B. The rectal temperatures of piglets experimentally infected with MRV3 F503 isolate were slightly higher compared to the control group (P>0.05) (FIG. 15A). However, there was no significant difference in the gross or microscopic intestinal lesions between the infected and control pigs. The MRV3-infected piglets derived from non-vaccinated sow had no significant gross or microscopic lesions, suggesting that the MRV3 F503 infected pigs but did not cause significant clinical disease, which contradicted the results of the previous study (Narayanappa et al., 2015,_supra). Recently, a Chinese MRV3 isolate also failed to cause diarrhea or vomiting in neonatal piglets experimentally-infected with a Chinese MRV-112013. See Qin, P., et al., Genetic and pathogenic characterization of a novel reassortant mammalian orthoreovirus 3 (MRV3) from a diarrheic piglet and seroepidemiological survey of MRV3 in diarrheic pigs from east China. Veterinary microbiology 208 (2017) 126-136.

In the present study, MRV3 RNA was detected in small intestinal contents from some of the MRV3-challenged piglets, but there was no statistical difference between virus-challenged and control groups at necropsy at 4 dpc (FIG. 15B). Additionally, the amounts of fecal viral RNA loads at 1, 3, and 4 dpc were similar among all virus-challenged pigs, suggesting that the virus replicated at a low level in infected pigs. In general, the number of piglets with detectable fecal viral RNA shedding and the amounts of viral RNA loads were higher at 4 dpc than in the earlier time points, suggesting that the virus did successfully infect the pigs, but replicated at a much lower level than that in the previous report (Narayanappa et al., 2015, supra). It is possible that the virus replication had not yet peaked at the time of necropsy at 4 dpc. Surprisingly, fecal viral RNA loads were higher in piglets born to sows that were vaccinated with 3 doses of the vaccine (FIG. 15C). Among the MRV3-challenged group, piglets from sows vaccinated with 2 or 3 doses of the inactivated MRV3 vaccine had significantly higher levels of MRV3-specific antibody than those from non-vaccinated sows (FIG. 16A), although the antibody response level is relatively low. There is no increase of the MRV3-specific antibody level in non-challenged piglets (p<0.01 at 0 dpc, p<0.001 at 4 dpc) (FIG. 16B). The short duration of the study of the infected piglets likely explains the low level of MRV3-specific antibody response. Overall, vaccination of sows with the inactivated MRV vaccine did not fully protect conventional piglets from the infection with the MRV3 virus.

All MRV3 F503 virus-infected conventional piglets survived and there was no mortality, no detectable diarrhea or other clinical signs of disease, and no statistical difference in weight gain in the challenge study. This result contradicted from results from the previous study (Narayanappa et al., 2015, supra) that severe clinical disease was observed in infected conventional pigs. It is possible that the low level of pre-existing MRV3 antibody in pregnant sows might have reduced the pathogenicity of MRV3 F503 in their offspring, as MRV3 antibody broadly exists in the pig population (Narayanappa et al., 2015, supra). Although we were able to select sows with the lowest level of the existing antibody for the present study, the low level of existing MRV3 maternal immunity transferred to pigs in this study might be responsible for the observed difference in pathogenicity. It is also quite possible that MRV3 causes only very mild disease in pig, but some unknown factor(s) or agent(s) in the piglets of the previous study may have enhanced the severity of the disease. Additionally, a major difference between these two studies is that, in the previous study (Narayanappa et al., 2015, supra), the neonatal pigs were separated from sows immediately after birth, and not fed with colostrum or sow milk. Therefore, the piglets from the previous study did not have an opportunity to acquire a sufficient level of maternal immunity, which may explain why those piglets infected with MRV3 developed severe disease. The neonatal piglets used in this study, however, were co-mingled with the sows allowing continuous suckling throughout the entire period of study. Furthermore, the virus stock used in this present study was a plaque-purified virus, whereas the virus used in the previously published study (Narayanappa et al., 2015, supra) was the lysate of cells infected with field fecal samples treated with chloroform. Thus, it cannot be completely ruled out the possibility of unknown agent(s) that may have contributed to the observed severe disease in the previous study.

Since, in this present study, we could not reproduce the severe disease associated with MRV3 infection in piglets that was reported previously (Narayanappa et al., 2015, supra), we decided to further evaluate the pathogenicity of MRV3 in a more sensitive model for pathogenicity study, the gnotobiotic pigs, which are colostrum-deprived and germ-free pigs. They are raised in sterile isolators and are not impacted by maternal immunity or adventitious infectious agents in conventional pigs, and thus are highly sensitive for pathogenicity study. The results showed that gnotobiotic pigs experimentally-infected with MRV3 F503 developed only very mild diarrhea at 7 dpc, and no severe disease was observed in infected pigs at all. Overall, the results from the gnotobiotic pig study are consistent with what we observed from the conventional pigs experimentally infected with MRV3 in this present study. Fecal virus shedding started from 2 to 4 dpc and peaked at 7 dpc with 6 out of the 8 gnotobiotic piglets having a high level of fecal MRV3 RNA loads (FIG. 17A). The MRV3 infection of gnotobiotic pigs did elicit a low level of MRV3 antibody (FIG. 17C), indicating that the MRV3 F503 did successfully infected gnotobiotic pigs and induced the virus-specific immune responses. There was no significant gross or histological lesions in the intestines, suggesting that the virus does not cause severe disease in pigs.

In summary, we demonstrate in this study that the plaque-purified MRV3 infected but did not induce severe disease in conventional piglets, which contradicts the previous report (Narayanappa et al., 2015, supra). The follow-up pathogenicity study of the MRV3 virus in the gnotobiotic pigs essentially confirmed our results with the conventional pigs, since we showed that the infected gnotobiotic pigs only developed very mild diarrhea at a late stage of infection. We also showed that maternal immunity in sows could partially protect against virus infection in offspring piglets, and that vaccination of pregnant sows with an inactivated MRV3 vaccine induced maternal immunity but did not fully protect piglets against MRV3 infection in conventional pigs. Taken together, the results from this study indicate that the MRV3 virus is not highly pathogenic in conventional or gnotobiotic pigs infected with this agent alone but that an inactivated viral vaccine was able to induce virus specific immune responses. Whether MRV3 can act as a trigger or co-factor with other known swine pathogens to exacerbate disease in the field remains unknown.

All publications, patents and patent applications cited herein are hereby incorporated by reference as if set forth in their entirety herein. While this invention has been described with reference to illustrative embodiments, this description is not intended to be construed in a limiting sense. Various modifications and combinations of illustrative embodiments, as well as other embodiments of the invention, will be apparent to persons skilled in the art upon reference to the description. It is therefore intended that the appended claims encompass such modifications and enhancements.

We claim:

1. A vaccine for protecting swine against porcine orthoreovirus type 3 (POV-3), comprising:
an attenuated or killed POV-3, the POV-3 having a σ1 capsid protein with at least 98% sequence homology to the σ1 capsid protein represented by SEQ ID NO: 20; and
a physiologically acceptable carrier, an adjuvant, or both.

2. The vaccine of claim 1, wherein the vaccine comprises the physiologically acceptable carrier.

3. The vaccine of claim 1, wherein the vaccine comprises the adjuvant.

4. The vaccine of claim 3, wherein the adjuvant is aluminum hydroxide, an immunostimulating complex, a non-ionic block polymer or copolymer, a cytokine, a saponin, monophosphoryl lipid A, a muramyl dipeptide, aluminum potassium sulfate, a heat-labile or heat-stable enterotoxin isolated from Escherichia coli, a cholera toxin or the B subunit thereof, a diphtheria toxin, a tetanus toxin, a pertussis toxin, or Freund's incomplete or complete adjuvant.

5. The vaccine of claim 1, wherein the vaccine is formulated for parenteral administration.

6. The vaccine of claim 5, wherein the vaccine is isotonic and pH buffered.

7. The vaccine of claim 5, wherein the vaccine comprises ethanol, propylene glycol, dextrose, an antioxidant, a chelating agent, or any combinations thereof.

8. The vaccine of claim 1, wherein the vaccine is formulated for intrabuccal or oral administration.

9. The vaccine of claim 1, wherein the vaccine comprises the attenuated POV-3.

10. The vaccine of claim 1, wherein the vaccine comprises the killed POV-3.

11. The vaccine of claim 1, wherein the vaccine comprises the attenuated or killed POV-3 in an amount effective to protect a swine from epidemic diarrhea caused by POV-3.

12. A method for immunizing a swine against POV-3, comprising administering to the swine the vaccine of claim 1.

13. The method of claim 12, wherein the swine is administered the vaccine of claim 3.

14. The method of claim 12, wherein the swine is administered the vaccine of claim 4.

15. The method of claim 12, wherein the swine is administered the vaccine orally, intrabuccally, intranasally, transdermally, or parenterally.

16. A method for making an antigen, comprising:
propagating a POV-3 having a σ1 capsid protein with at least 98% sequence homology to the σ1 capsid protein represented by SEQ ID NO: 20 in a cell culture, in an embryonated chicken egg, or both.

17. The method of claim 16, wherein the cell culture is non-porcine, and wherein the POV-3 is propagated until the POV-3 is attenuated.

18. The method of claim 16, wherein the cell culture is non-porcine, and wherein the POV-3 is propagated until the POV-3 is capable of conferring immunity but incapable of causing epidemic diarrhea when administered to a swine.

19. The method of claim 16, wherein the POV-3 is propagated in the cell culture.

20. The method of claim 16, further comprising inactivating the propagated POV-3.


SEQUENCE LISTING (56 sequences)

SEQ ID NO: 1 - DNA, 20 bp, Orthoreovirus
tgggacaacttgagacagga

SEQ ID NO: 2 - DNA, 20 bp, Orthoreovirus
ctgaagtccaccrttttgwa

SEQ ID NO: 3 - DNA, 32 bp, Orthoreovirus
cactctgatacaatccttaggatcactcaagg

SEQ ID NO: 4 - DNA, 30 bp, Orthoreovirus
ccatcgtcatacgattgttattgattgcca

SEQ ID NO: 5 - DNA, 30 bp, Orthoreovirus
ctatactagctgacacttcgatgggattgc

SEQ ID NO: 6 - DNA, 26 bp, Orthoreovirus
cgtctcatccatttctgccagctctt

SEQ ID NO: 7 - DNA, 3854 bp, Orthoreovirus
gctacacgttccacgacaatgtcatccatgatactgactcagtttggaccgttcattgaaagcatctcaggtatcactgaccaatcgaacgacgtgtttgaagatgcagcaaaagcattctctacgtttactcgcagcgacgtctataaggcgctggatgagatacctttctctgatgacgcgatgcttcccatccctccaactatatataccaaaccatctcacgattcatattattacatagatgctctaaaccgcgtacgtcgtaaaacatatcagggccctgatgacgtgtacgtacctaattgttccatcgttgaattgctagagccgcatgagactctgacatcttatgggcgtttgtctgaagcgattgagaatcgtgccaaggatggagacagccaagccagaattgcgacaacatacggtagaatcgctgagtctcaggctagacagattaaggctccattggagaagtttgtgttggcactattggtgtccgaagcggggggttctctatatgacccagttttgcagaagtatgatgagattccagatctatcgcataattgccctttatggtgttttagagaaatctgtcgtcacatatctggtccattaccagatcgagcaccttatctttacttatcggcaggggttttctggttaatgtcaccacggatgacgtctgcgatccctccgttattatctgatcttgttaatttagctatcttacaacagactgcaggtttagatccatcattagtgaaattgggagtgcagatatgtcttcacgcagcagctagttcgagttatgcatggtttatcctaaagactaagtctatttttcctcaaaacacgttacatagtatgtatgagtctctagaaggagggtactgtcctaacctagaatggttagagcctagatcggactataaatttatgtacatgggagtcatgccattgtccactaaatatgctaggtcggcaccatccaacgaaaagaaagcgcgggaacttggtgagaaatatggattgagttcagttgtcagtgagcttcgtaaacggacaatggcttatgttaaacatgactttgcttcggtaaggtacattcgtgacgccatggcatgtactagcggcatttttctggtaagaacacccaccgagacggtattgcaagaatatacccaaagtccggagattaaggttcccatcccccacaaagactggacaggcccagtaggtgaaatcagaattctaaaagatacaaccagctccatcgcgcgctacttgtatagaacatggtacttagcagcggcaagaatggcggctcagccacgcacgtgggatccattgttccaggcgattatgagatctcaatacgtgacagctaggggtgggtctggcgcagcactccgcgaatctctgtatgcaattaatgtgtcgttacctgattttaagggcttaccagtgaaggcagcaactaagatatttcaggcggcacaattagcgaacctgccgttctcacacacatcagtggctatactagctgacacttcgatgggattgcgaaaccaggtgcagaggcgaccacgatccatcatgcccttaaatgtgccccaacagcaggtttcggcgcctcatacattgaccgctgattatatcaattatcacatgaatctatcgactacgtctggtagcgcggtcattgagaaagtgattcctttaggtgtatacgcttcaagccctcctaaccaatcgattaacattgacatatctgcgtgcgacgcaagtattacttgggacttctttctatccgtgattatggcggctatacacgaaggtgtcgctagtagctccattggaaaaccgttcatgggagttcctgcatccatcgtaaatgatgagtctgtcgttggagtgagagctgctaggccgatatcgggaatgcagaacatggttcagcatctatcaaaactgtacaaacgtggattttcatatagagtgaacgactctttttctccaggcaacgattttactcatatgactaccactttcccgtcaggttcaacagccacttctactgagcatactgccaataatagtacgatgatggaaactttcctgacagtatggggacccgaacatactgacgaccccgacgtcttacgtctaatgaagtctttgactattcaaaggaattacgtgtgtcaaggtgatgatggattgatgattatcgatgggaatactgctggtaaggtgaaaagtgaaactgttcagaagatgttggagttaatctcaaaatatggtgaggagtttggatggaaatatgacatagcgtacgatgggactgccgagtacctaaagctgtacttcatatttggctgtcgaattccaaatcttagccgtcatccaattgttggaaaagaacgggcgaattcttcagcagaggagccatggccagcaattttagatcagattatgggtatcttctttaatggcgttcatgacgggttgcagtggcagcggtggatacgttattcatgggctctatgctgtgctttctcacgccaaaggacaatgattggcgagagcgtgggttacattcaatatcctatgtggtcatttgtctactggggattaccattggtaaaagtgttcgggtcagacccatggatattctcttggtacatgccgactggggacttgggaatgtatagttggattagcctaatacgccctctaatgacaagatggatggtagctaatggctatgtcactgacaaatgctcacccgtattcgggaacgcagattatcgtaaatgtttcaatgagattaaattatatcaagggtattatatggcacaattgcccaggaatcccacaaaatctggacgagcggcccctcgggaggtgagagaacaatttactcaggcactatctgattatctgatgcaaaatccagaactgaagtcacgtgtgctacgtggtcgtagtgagtgggagaagtatggagccgggataattcacaaccctccatcattattcgatgtcccccataagtggtatcagggtgcgcaagaggcggcgaccgctacgagagaagagctggcagaaatggatgagacgttgatacgcgcccgaaggcacagttattcgagtttctcaaaattgttggaggcatacctgcttgtgaaatggcgaatgtgcgaggcccgcgaaccgtcggttgatttgcgattaccattgtgtgcgggtattgacccactaaactcagatccttttctcaaaatggtaagcgttggaccgatgcttcagagtacgcgaaagtactttgctcagacactattcatggcgaaaacggtgtcgggtctcgacgttaacgcgattgatagcgcgttattacgactgcgaacattgggcgctgataagaaagcattaacagcgcagttattaatggtgggacttcaggagtcagaggcggatgcgttggctgggaagataatgttgcaagatgtaagtactgtgcaattagctagagtggtcaatttagcggtgccagatacgtggatgtcgttggattttgattctatgttcaaacaccatgttaaattgcttcccaaagatggacgccacctaaatactgacattcctcctcgcatgggatggttacgggccattctacgattcctaggtgctggaatggtaatgactgcgactggagttgctgtcgacatatatctggaggatatacacggtggtggtcgatcacttggacagagattcatgacttggatgcggcaggaaggacggtcagcgtgagtctaccatgggtcgtggtgcgtcaactcatc

SEQ ID NO: 8 - protein, 1267 aa, Orthoreovirus
MSSMILTQFGPFIESISGITDQSNDVFEDAAKAFSTFTRSDVYKALDEIPFSDDAMLPIPPTIYTKPSHDSYYYIDALNRVRRKTYQGPDDVYVPNCSIVELLEPHETLTSYGRLSEAIENRAKDGDSQARIATTYGRIAESQARQIKAPLEKFVLALLVSEAGGSLYDPVLQKYDEIPDLSHNCPLWCFREICRHISGPLPDRAPYLYLSAGVFWLMSPRMTSAIPPLLSDLVNLAILQQTAGLDPSLVKLGVQICLHAAASSSYAWFILKTKSIFPQNTLHSMYESLEGGYCPNLEWLEPRSDYKFMYMGVMPLSTKYARSAPSNEKKARELGEKYGLSSVVSELRKRTMAYVKHDFASVRYIRDAMACTSGIFLVRTPTETVLQEYTQSPEIKVPIPHKDWTGPVGEIRILKDTTSSIARYLYRTWYLAAARMAAQPRTWDPLFQAIMRSQYVTARGGSGAALRESLYAINVSLPDFKGLPVKAATKIFQAAQLANLPFSHTSVAILADTSMGLRNQVQRRPRSIMPLNVPQQQVSAPHTLTADYINYHMNLSTTSGSAVIEKVIPLGVYASSPPNQSINIDISACDASITWDFFLSVIMAAIHEGVASSSIGKPFMGVPASIVNDESVVGVRAARPISGMQNMVQHLSKLYKRGFSYRVNDSFSPGNDFTHMTTTFPSGSTATSTEHTANNSTMMETFLTVWGPEHTDDPDVLRLMKSLTIQRNYVCQGDDGLMIIDGNTAGKVKSETVQKMLELISKYGEEFGWKYDIAYDGTAEYLKLYFIFGCRIPNLSRHPIVGKERANSSAEEPWPAILDQIMGIFFNGVHDGLQWQRWIRYSWALCCAFSRQRTMIGESVGYIQYPMWSFVYWGLPLVKVFGSDPWIFSWYMPTGDLGMYSWISLIRPLMTRWMVANGYVTDKCSPVFGNADYRKCFNEIKLYQGYYMAQLPRNPTKSGRAAPREVREQFTQALSDYLMQNPELKSRVLRGRSEWEKYGAGIIHNPPSLFDVPHKWYQGAQEAATATREELAEMDETLIRARRHSYSSFSKLLEAYLLVKWRMCEAREPSVDLRLPLCAGIDPLNSDPFLKMVSVGPMLQSTRKYFAQTLFMAKTVSGLDVNAIDSALLRLRTLGADKKALTAQLLMVGLQESEADALAGKIMLQDVSTVQLARVVNLAVPDTWMSLDFDSMFKHHVKLLPKDGRHLNTDIPPRMGWLRAILRFLGAGMVMTATGVAVDIYLEDIHGGGRSLGQRFMTWMRQEGRSA

SEQ ID NO: 9 - DNA, 3915 bp, Orthoreovirus
gctattggcgcaatggcgaacgtttggggagtgagacttgcagactctttatcgtcacccactattgagacaagaactcgtcattacacactccgcgatttctgttccgacctggatgctgtagttggcaaggaaccctggagacccttacgcaatcagagaacgaatgatattgtcgccgttcaattgtttcggccactgcagggattggtgcttgacacgcagttttatggattccctggcattttctcagaatgggaacagtttataagagagaaactacgcgtgttgaaatatgaagttttgcggatttacccgatcagtaattataatcatgagcgtgtcaatgtcttcgtggcaaatgctcttgtcggtgcatttctatccaaccaagccttctatgacctgttgcctctattattaatacgtgataccatgataaatgacttacttgggacaggtgctgctctttctcagtttttccaatctcatggtgaggttttagaggttgccgcaggaaggaagtacctgcaaatgaagaactactcgaacgatgatgatgatccacctttattcgctaaggatctgtcggattatgcgaaggcgttttacagtgatacgtttgagactttagaccgattcttctggacacatgactcatctgcgggcgtcctagtgcattatgataagcctaccaatgggaatcattacatcttgggtactctgacgcagatggttagtgcgcctccgcatatcattaacgctactgacgcattgttgctcgaatcgtgtttagaacaatttgcggagaatgtgagagccaggccagcgcagcctgttccaagattggatcagtgttaccatttacggtggggtgctcaatatgttggcgaggactcattgacgtaccgtttgggggtactttcactactggctaccaacggatatcaattagctagaccgatccctaagcagttaacgaatcgatggctttctagttttgtcagtcagataatgtcggatggtgtgaatgagacgccattatggcctcaagagagatatgtccaaatagcctacgattcaccgtctgtagtcgacggagctacgcactatggttatgttaggagaaatcagttgcggttgggcatgagggtgtccgctcttcagtcattgagtgatactccggctccgatacagtggttaccgcagtatactattgatcaggcacctgttgatgagggagatctaatggtttcgcggttgactcaactaccgttacgccctgattatggtagcatatgggtcggtgacgctctatcgtattatgttgattacaaccgcagccatagagttgtactatcatccgagctaccacaactaccagatacatactttgacggagacgagcaatacggtcgcagtctgttctctttagcacgaaaaatcggtgatcgatctctcatcaaagatacagcagtgctcaagcatgcgtaccaggccatcgatccaaacactggaaaggaataccttcgcgcaggacagtctgttgcatatttcggagcatcagctggtcattcaggggcggatcaacctctagtaattgagccatggacgcagggtaaaattagtggtgtaccgcagccttcttcagtcagacagtttgggtatgatgttgctaaaggtgcgattgtggacttagcaagaccgttcccgtcgggtgactaccaatttgtatattctgacgtcgatcaggtcgttgacggccacgatgatctcagcatatcttcagggctggtggagagtctattagattcctgcatgcatgccacatccccaggtgggtcgttcgtgatgaagataaatttcccgacacgtgatgtctggcactatatagagcaaaagattctcccaaatattacctcgtacatgttgatcaaaccattcgtgactaacaatgtagagttattctttgtggctttcggtgtgcatcaacaatcagcattgacatggacgtccggggtgtatttcttcctggtcgatcacttctatcgatacgagacattgtctacgatttcacgtcagttgccatcgttcggatacgttgatgacgggtcgtctgtgacaggtattgagatgatcagtcttgaaaatccaggcttttcaaacatgacccaagctgcacgtgtcgggatatcagggctgtgtgcgaatgtcggtaatgcgcgcaaattaatatctatccatgaatctcacggagcacgcgtgctcaccatcatatcgagaagatctccggcttcggctaggcggaaagctcgcttacgctatttgccactcatagacccacgatctttggaagtgcaggcacgtacgatattaccatctaacccagtgctgtttgacaacgtaaaaggagcatcgcctcacgtatgtttgacgatgatgtataactttgaagtatctagtgcggtgtatgatggtgatgtagtgcttgaccttggtaccggtcctgaagcgaagattctggagctgattcctccaacgtccccagtaacatgcgtggacattagaccgacggcacagcctagtggctgttggaacgtacgtacgacatttctggagcttgattacctaagtgatggctggataacgggtgtacgtggcgacatcgtgacctgcatgctgtccctgggtgctgctgctgctggaaaatccatgacgttcgacgcggcatttcaacagttagtgaaagtgcttactaaaagtacagctaacgtactgctgatccaagtcaactgcccaacggatgtaatccgaacaattaagggatatttggagatagatcaaactaataagcggtatagatttcccaaatttggccgtgatgaaccatactctgacatggattccttagagcgcatatgtcgtgctgcgtggccaaattgttccatcacgtgggtgcctttatcctatgatctacgttggactaaacttgctttgcttgaatcgactacactgagcagtgcatcagtgagaattgctgagttgatgtacaaatacatgccagttatgaggatagatattcatgggttacccatggaaaagcaaggcaatttcgtagtgggtcagaactgttctctaactataccgggcttcaacgcacaggacgtgttcaactgctacttcaattccgcgctcgctttctctactgaggatgttaattcggcaatgataccacaagtgacggctcagtttaacactagtaaaggtgagtggtcattggacatggtgttctcagacgctggtatctacacaatgcaggcattagtaggttccaacgcaaatcctgtgtctttgggttcgtttgtagtggattctccggatgtcgacataacagatgcgtggcctgctcagttagattttaccatagctggcactgatgtcaacatcacagttaatccttattaccgcttgatggcctttgtaaagattgatggacaatggcagattgcgaaccctgataaattccaatttttctcatcaggtacagggacgttagtgatgaatgtaaagttagatatagctgataggtatttgctatattacattcgcgacgttcaatctagggatgtgggattttacatacagcacccattacagttattaaatacaattacgttgcctacaaacgaggatttattcttgagcgctcctgacatgcgcgagtgggcggtaaaggaaagtggcaataccatatgcatacttaatagccagggttttgtgccacctcaggattgggatgttcttaccgacactattagctggtctccttcgctcccaacttatgtggtacctccgggtgattatactctgacacctctgtaactcattacccctcgtaagcgtgcctaattcatc

SEQ ID NO: 10 - protein, 1289 aa, Orthoreovirus
MANVWGVRLADSLSSPTIETRTRHYTLRDFCSDLDAVVGKEPWRPLRNQRTNDIVAVQLFRPLQGLVLDTQFYGFPGIFSEWEQFIREKLRVLKYEVLRIYPISNYNHERVNVFVANALVGAFLSNQAFYDLLPLLLIRDTMINDLLGTGAALSQFFQSHGEVLEVAAGRKYLQMKNYSNDDDDPPLFAKDLSDYAKAFYSDTFETLDRFFWTHDSSAGVLVHYDKPTNGNHYILGTLTQMVSAPPHIINATDALLLESCLEQFAENVRARPAQPVPRLDQCYHLRWGAQYVGEDSLTYRLGVLSLLATNGYQLARPIPKQLTNRWLSSFVSQIMSDGVNETPLWPQERYVQIAYDSPSVVDGATHYGYVRRNQLRLGMRVSALQSLSDTPAPIQWLPQYTIDQAPVDEGDLMVSRLTQLPLRPDYGSIWVGDALSYYVDYNRSHRVVLSSELPQLPDTYFDGDEQYGRSLFSLARKIGDRSLIKDTAVLKHAYQAIDPNTGKEYLRAGQSVAYFGASAGHSGADQPLVIEPWTQGKISGVPQPSSVRQFGYDVAKGAIVDLARPFPSGDYQFVYSDVDQVVDGHDDLSISSGLVESLLDSCMHATSPGGSFVMKINFPTRDVWHYIEQKILPNITSYMLIKPFVTNNVELFFVAFGVHQQSALTWTSGVYFFLVDHFYRYETLSTISRQLPSFGYVDDGSSVTGIEMISLENPGFSNMTQAARVGISGLCANVGNARKLISIHESHGARVLTIISRRSPASARRKARLRYLPLIDPRSLEVQARTILPSNPVLFDNVKGASPHVCLTMMYNFEVSSAVYDGDVVLDLGTGPEAKILELIPPTSPVTCVDIRPTAQPSGCWNVRTTFLELDYLSDGWITGVRGDIVTCMLSLGAAAAGKSMTFDAAFQQLVKVLTKSTANVLLIQVNCPTDVIRTIKGYLEIDQTNKRYRFPKFGRDEPYSDMDSLERICRAAWPNCSITWVPLSYDLRWTKLALLESTTLSSASVRIAELMYKYMPVMRIDIHGLPMEKQGNFVVGQNCSLTIPGFNAQDVFNCYFNSALAFSTEDVNSAMIPQVTAQFNTSKGEWSLDMVFSDAGIYTMQALVGSNANPVSLGSFVVDSPDVDITDAWPAQLDFTIAGTDVNITVNPYYRLMAFVKIDGQWQIANPDKFQFFSSGTGTLVMNVKLDIADRYLLYYIRDVQSRDVGFYIQHPLQLLNTITLPTNEDLFLSAPDMREWAVKESGNTICILNSQGFVPPQDWDVLTDTISWSPSLPTYVVPPGDYTLTPL

SEQ ID NO: 11 - DNA, 3901 bp, Orthoreovirus
gctaatcgtcaggatgaagcggattccaaggaagacaaagggcaaatccagcggaaagggcaatgactcgacagatagagcggacgatggctcgagccaattacgagataagcaaaacaataagaccggccccgccactgcagagcctggaacgtccaaccgagagcgatacaaagctcgaccaagtattgcatctgtgcagagggccactgaaagtgcagaactgcccatcaagaataatgacgaaggaacgccagataagaaaggaaatactaagggcgacttagttggtgggcatagtgaggctaaagatgaggcggatgaagcgacgaagaagcaggcaaaagatacagataaaagtaaagcgcaagtcacatattcagacactggtatcaataatgctaatgaactgtcaagatctgggaatgtggataatgagggtggaagtaatcagaaaccgatgtccaccagaatagctgaagcaacgtctgctatagtgtctaaacatcctgcgcgtgttgggttaccacctaccgctagcagtggtcatgggtatcagtgtcatgtctgttctgcagtcctgttcagtcctttagacctagacgcccacgtcgcatcacatggtttacatggtaatatgacgttgacgtcgagtgagattcagcgacatatcactgagtttattagttcatggcaaaatcatcctattgttcaagtttcggctgacgtcgaaaataagaagactgctcagttgcttcacgctgatactcctcgacttgtcacttgggatgctggtctgtgtacttcgttcaaaatcgtcccaattgtaccagctcaggtgccgcaggatgtactggcctatacgttcttcacctcttcatatgctattcaatcaccgtttccagaggcggcggtgtctaggattgtggtgcatacaagatgggcatctaatgttgactttgaccgagattcatctgtcatcatggcaccacctacagaaaataatatccacttgtttaagcagttgctgaatactgataccctgtctgtgaggggggccaacccactaatgtttagggcgaacgtattgcatatgttgctggagttcgtattggataacttgtatttgaacagacatacgggattctctcaagaccacacaccattcactgagggcgctaatctgcgttcacttcctggccccgatgctgagaaatggtattcgatcatgtatcccacgcgcatgggaacgccgaatgtatcgaaaatatgtaatttcgtcgcctcttgtgtgcgaaatcgagtaggaaggtttgatcgagcacagatgatgaacggagccatgtcagagtgggtggatgtcttcgagacttcagacgcgcttaccgtctccattcggggtcgatggatggctagactggctcgcatgaacataaatccgacagagatcgaatgggcgttgactgaatgtgcacaaggatatgtgactgttacaagtccttacgctcctagcgtaaatagattgatgccatatcgtatttccaacgctgagcggcagatatcacagataatcaggatcatgaacattggcaataatgccacggtgatacaacccgtcctacaagatatttcggtactccttcaacgcatatcaccactccaaatagatccaaccattatttctaacactatgtcaacagtctcggagtctactactcagacactcagccccgcgtcctcaattttgggtaaactacgaccgagtaactcagatttctctagttttagagtcgcgttggctgggtggctttataatggagttgtgacgacggtgatcgatgatagttcatatccaaaggacggtggcagcgtgacctcacttgaaaatctgtgggattttttcatccttgcgcttgctctaccactgacaactgacccatgtgcacctgtgaaagcgttcatgactttagctaacatgatggtcggtttcgagacgatccccatggataatcagatctatactcaatcgagacgcgcgagtgctttctcaacgcctcacacgtggccacgatgtttcatgaatatccagttaatttctcccatcgatgctcccatattacgacagtgggctgaaattattcatagatactggcctaatccttcacagattcgttatggtgcaccgaacgtctttggctcggcaaatctgttcactccacctgaggtgctgttattgccaatcgatcatcaaccagctaatgtgacaacgccaacgctggacttcaccaacgaattgactaattggcgtgttcgcgtttgtgagcttatgaagaatcttgttgataatcaaagatatcaacctggatggacacaaagtctagtctcgtcaatgcgcggaacgctggacaaactgaaattgatcaaatcgatgacaccaatgtatctgcaacagctggctccggtagagttagcagtgatagcccccatgttgccttttccacctttccaagtgccttacgtccgccttgatcgtgatagagttccaacaatggtcggagtgacacgacagtcacgagatactatcactcagccagcgctatcattgtcaacaaccaataccactgttggtgtgcctctagctctagacgcaagggctattaccgttgcgctgttgtcagggaaatatccgccggatttggtgacaaatgtatggtacgctgatgccatttatccaatgtatgcagatactgaggtgttctctaatcttcagagagacatgattacctgcgaagccgtgcagacattagtgactctggtggcgcagatatcagagacccagtatcctgtagataggtatcttgattggatcccatcactgagagcatcggcggcgacggcggcgacatttgctgagtgggttaatacttcaatgaagacggcgtttgatttgtctgacatgctgttagaacctctcctaagcggagatccgaggatgactcaactagcgattcagtatcagcaatacaatggcagaacgtttaatgtcatacctgaaatgccaggttcagtcattgctgactgtgttcaactaacagcagaagtctttaatcacgaatataacctgtttgggattgcgaggggtgatatcatcattggtcgtgtccagtcgacacacttgtggtcaccactggctcctccacctgacctggtgttcgatcgtgatactcctggcgttcacatcttcggacgagattgccgtatatcgtttggaatgaatggcgccgcgccaatgattagagatgagactggaatgatggtgcctttcgaaggaaattggatttttccactggcgctttggcaaatgaatacacgatattttaatcaacagttcgacgcgtggattaagacaggagagttgcgaatccgtattgagatgggcgcgtatccatatatgttgcattactatgatccacgtcagtacgctaatgcatggaatttgacatccgcctggcttgaagaaattacaccgacgagcattccatccgtgcctttcatggtaccaatttcaagtgatcatgacatttcctctgccccagctgtccaatatatcatttcgactgaatataatgatcggtctttattctgcactaactcatcatctccccaaaccatcgctggaccagacaaacacattccagttgaaagatataacattctgaccaaccccgatgctccacccacgcagatacaactgcctgaagttattgacttgtataacgtcgtcacacgctatgcgtatgagactccacctattaccgctgttgttatgggtgttccttgatcctcatcctcccaacaggtgctagagcatcgcgctcgatgctagttgggccgattcatc

SEQ ID NO: 12 - protein, 1275 aa, Orthoreovirus
MKRIPRKTKGKSSGKGNDSTDRADDGSSQLRDKQNNKTGPATAEPGTSNRERYKARPSIASVQRATESAELPIKNNDEGTPDKKGNTKGDLVGGHSEAKDEADEATKKQAKDTDKSKAQVTYSDTGINNANELSRSGNVDNEGGSNQKPMSTRIAEATSAIVSKHPARVGLPPTASSGHGYQCHVCSAVLFSPLDLDAHVASHGLHGNMTLTSSEIQRHITEFISSWQNHPIVQVSADVENKKTAQLLHADTPRLVTWDAGLCTSFKIVPIVPAQVPQDVLAYTFFTSSYAIQSPFPEAAVSRIVVHTRWASNVDFDRDSSVIMAPPTENNIHLFKQLLNTDTLSVRGANPLMFRANVLHMLLEFVLDNLYLNRHTGFSQDHTPFTEGANLRSLPGPDAEKWYSIMYPTRMGTPNVSKICNFVASCVRNRVGRFDRAQMMNGAMSEWVDVFETSDALTVSIRGRWMARLARMNINPTEIEWALTECAQGYVTVTSPYAPSVNRLMPYRISNAERQISQIIRIMNIGNNATVIQPVLQDISVLLQRISPLQIDPTIISNTMSTVSESTTQTLSPASSILGKLRPSNSDFSSFRVALAGWLYNGVVTTVIDDSSYPKDGGSVTSLENLWDFFILALALPLTTDPCAPVKAFMTLANMMVGFETIPMDNQIYTQSRRASAFSTPHTWPRCFMNIQLISPIDAPILRQWAEIIHRYWPNPSQIRYGAPNVFGSANLFTPPEVLLLPIDHQPANVTTPTLDFTNELTNWRVRVCELMKNLVDNQRYQPGWTQSLVSSMRGTLDKLKLIKSMTPMYLQQLAPVELAVIAPMLPFPPFQVPYVRLDRDRVPTMVGVTRQSRDTITQPALSLSTTNTTVGVPLALDARAITVALLSGKYPPDLVTNVWYADAIYPMYADTEVFSNLQRDMITCEAVQTLVTLVAQISETQYPVDRYLDWIPSLRASAATAATFAEWVNTSMKTAFDLSDMLLEPLLSGDPRMTQLAIQYQQYNGRTFNVIPEMPGSVIADCVQLTAEVFNHEYNLFGIARGDIIIGRVQSTHLWSPLAPPPDLVFDRDTPGVHIFGRDCRISFGMNGAAPMIRDETGMMVPFEGNWIFPLALWQMNTRYFNQQFDAWIKTGELRIRIEMGAYPYMLHYYDPRQYANAWNLTSAWLEEITPTSIPSVPFMVPISSDHDISSAPAVQYIISTEYNDRSLFCTNSSSPQTIAGPDKHIPVERYNILTNPDAPPTQIQLPEVIDLYNVVTRYAYETPPITAVVMGVP

SEQ ID NO: 13 - DNA, 2304 bp, Orthoreovirus
gctattcgcggtcatggcttacatcgcagttcctgcggtggtggattcacgttcgagtgaggctattggactactagaatcgtttggagtagacgctggggctgatgtgaatgatgtttcatatcaagatcatgactatgtgttggatcagttacagtatatgttagatgggtatgaggctggtgacgtcatcgatgcactcgtccacaagaattggttacatcattctgtctattgcttgttgccacccaaaagtcaactactagagtattggaaaagtaacccttcagcgataccggacaacgttgatcgtcggcttcgtaaacggctaatgctaaagaaagatctcagaaaagatgatgagtacaatcaattggcgcgtgctttcaagatatcggatgtctacgcaccactcatctcatctacgacgtcaccgatgacaatgatccagaacttgaatcagggcgagatcgtgtacaccacgacggacagagtaattggggctagaatcttgttatatgctccaagaaagtactatgcatcaactctatcatttactatgactaagtgcatcattccgtttggcaaagaggtgggccgtgctcctcactctagatttaatgttggcacattcccatcaattgctactccgaagtgttttgttatgagtggggttgatattgagtccatcccaaatgaatttatcaaattgttttaccagcgcgtcaagagtgttcacgctaatatactaaatgacatatcacctcagatactctctgacatgataaacagaaagcgtttgcgtgttcatactccatcagatcgtcgagccgcgcaactgatgcatttgccctatcatgttaagcgaggggcgtctcacgtcgacgtttataaggtagatgttgtggatgtattgtttgaggtagtagatgtggccgatgggttgcgcaatgtatctaggaagctaactatgcacactgttccggtctgtattcttgaaatgttgggtattgagattgcggactattgcgttcgtcgagaggatggaatgttcacagattggttcttgcttttaaccatgctatctgatggcttaactgatagaaggacgcgttgtcaatacctgattaatccgtcaagcgtgcctcctgatgtaatacttaacatctctattactggatttataaacaggcatacaatcgacgtcatgcctgacacatacgacttcattaaacccattggtgctgtgctgcctaagggatcattcaaatcgacaattatgagagttcttgactcaatatcaatattaggagttcagatcatgccgcgcacgcatgtagtcgactcggatgaggtgggcgagcaaatggagcctacgtttgagcatgcggtcatggagatatacagaggaattgctggcgttgactctctggatgatctcattaggtgggtgctgaactcggatctcattccatatgatgacaggcttggccaattatttcaagcgtttctgcctctcgcaaaagatttgttagcgccaatggccagaaagttttatgataactcaatgagtgagggtagattgctgacattcgctcatgctgatagtgagttgctgaacgcaaattactttggtcatttactgcgactaaaaataccatatattacagaggttaatttgatgattcgcaagaatcgtgagggtggggagctatttcagcttgtgttatcacatctatataaaatgtatgctactagcgcgcagcctaaatggtttggatcattattgcgattgttaatatgtccctggttacatatggagaaattgataggagaagcagacccagcatctacgtcggctgaaattggatggtatatctctcgtgaacagctgatgcaagatggatggtgtggatgtgaagatggattcattccctatattagcatacgtgcgccaaagctggttatagaggagttaatggagaagaattggggccaatatcatgcacaagttattatcactgatcggcttgtcgtaggcgaaccgcgtagggtatctgccaaggctgtggtcaaaggtaaccacttaccagttaagttagtctcacgatttgcatgtttcacactgacgacgaagtatgagatgaggctttcatgtggccatagcactggacggggggctgcatacaatgcgagactagttttccgatctgacttggcgtgatccgtgacatgcgtagtgtgacacctgcccctaggtcaatgggggtagggggcgggctaggactacgtacgcgcttcatc

SEQ ID NO: 14 - protein, 736 aa, Orthoreovirus
MAYIAVPAVVDSRSSEAIGLLESFGVDAGADVNDVSYQDHDYVLDQLQYMLDGYEAGDVIDALVHKNWLHHSVYCLLPPKSQLLEYWKSNPSAIPDNVDRRLRKRLMLKKDLRKDDEYNQLARAFKISDVYAPLISSTTSPMTMIQNLNQGEIVYTTTDRVIGARILLYAPRKYYASTLSFTMTKCIIPFGKEVGRAPHSRFNVGTFPSIATPKCFVMSGVDIESIPNEFIKLFYQRVKSVHANILNDISPQILSDMINRKRLRVHTPSDRRAAQLMHLPYHVKRGASHVDVYKVDVVDVLFEVVDVADGLRNVSRKLTMHTVPVCILEMLGIEIADYCVRREDGMFTDWFLLLTMLSDGLTDRRTRCQYLINPSSVPPDVILNISITGFINRHTIDVMPDTYDFIKPIGAVLPKGSFKSTIMRVLDSISILGVQIMPRTHVVDSDEVGEQMEPTFEHAVMEIYRGIAGVDSLDDLIRWVLNSDLIPYDDRLGQLFQAFLPLAKDLLAPMARKFYDNSMSEGRLLTFAHADSELLNANYFGHLLRLKIPYITEVNLMIRKNREGGELFQLVLSHLYKMYATSAQPKWFGSLLRLLICPWLHMEKLIGEADPASTSAEIGWYISREQLMQDGWCGCEDGFIPYISIRAPKLVIEELMEKNWGQYHAQVIITDRLVVGEPRRVSAKAVVKGNHLPVKLVSRFACFTLTTKYEMRLSCGHSTGRGAAYNARLVFRSDLA

SEQ ID NO: 15 - DNA, 2205 bp, Orthoreovirus
tgctaatctgctgaccgttactctgcaaagatggggaacgcttcctctattgttcagacgatcaacgtcactggagatggcaatgtgttcaaaccctcagctgagacttcatccaccgctgtaccgtcactaagtctatcacctggaatgctaaatcctggaggagtaccatggatcgcgattggggatgagacatctgttacttcaccgggtgcgttgcggcgaatgacttcgaaggatattccagaaacagcgataatcaacacagataattcatcaggcgcggtgccaagtgaatcagcgttggtgccttacaatgatgagccattggtggtggtgacggagcatgctatcgcaaactttactaaagctgagatggcacttgaattcaatcgtgagtttcttgataaattgcgcgtactgtcagtgtcaccgaaatattctgaccttctaacgtatgttgattgctacgttggtgtgtcggctcgtcaagccctaaacaatttccagaaacaggtacctgtgattacacctactagacaaacaatgtatgttgactccatacaggcggccttgaaagcccttgagaaatgggaaattgatttgagagtggctcagacgctgttgcctacaaatgtcccaattggggaggtttcttgtccaatgcagtcagtagtgaaactattagatgatcagctgcccgacgatagccttatacgaaggtatcctaaggaggctgctgttgctttggccaaaaggaacgggggaatacagtggatggatgtgtcagaaggtactgtgatgaacgaggccgtaaatgctgttgcagcaagtgccctggcaccttccgcctcatccccgcccctggaagagaaatcaaaattgactgagcaagcgatggatcttgtaaccgcagctgaacctgagatagtcgcctctctcgtgccagttccagcgcccgtgtttgccattccacctaagccagccgattataacgtgcgtaccctgaagatcgatgaggccacatggttgcgaatgattccaaaaactatgagtacgcctttccaaattcaagtgactgataatacaggaactaaatggcatcttaacttgagaggagggacacgcgtagtgaatctggaccagattgctccgatgaggttcgttctggatctagggggaaagagttacaaggagacgagttgggatccaaacggtaagaaggttgggtttatcgtattccagtctaagattccttttgagctttggaccgctgcatcacagattggtcaagccacagtggtcaactatgttcagctatatgctgaagacagctcatttaccgcccagtctattatcgctactacatcgttggcttataattatgaaccagagcaattgaataagactgaccctgaggtgaactattaccttctagcgacttttatagattcagctgctataacaccgacgaacatgacacagcctgatgtttgggatgctatgttgacgatgtctccattgtccgctggggaggtgactgtgaagggtgcggtggtaagcgaggtggtgccagcggaattgatcggcagctatactccagagtcattaaatgcctcacttccgaatgacgctgctagatgtatgattgatagagcctcgaaaatagccgaagctataaagattgatgatgacgctgggccagatgaatactctcccaactctgtaccaattcaaggtcagttggctatttctcaacttgagactgggtatggtgtacggatattcaattctaagggaattctttcgaaaatcgcgtccagagctatgcaggcttttatcggtgatccaagcacaattatcacgcaggcggcaccagtgctgtcagataagaacaattggattgcattggcacaaggagtcaagactagtttgcgtaccaaaagtctatcagcgggggtgaagacggcggtgagtaaactgagctcgtccgagtctattcagagttggactcaaggattcttggataaagtatcgatgcattttccagcgcctaagtcggactgtccgaccagcggagatagcagtgaatcgtccgctcggcgagtgaagcgcgactcatacgcaggagtggttaagcgtgggtatacacgttaagccgctcgccctggtgacgcggggttaagggatgcaggcacatcatca

SEQ ID NO: 16 - protein, 708 aa, Orthoreovirus
MGNASSIVQTINVTGDGNVFKPSAETSSTAVPSLSLSPGMLNPGGVPWIAIGDETSVTSPGALRRMTSKDIPETAIINTDNSSGAVPSESALVPYNDEPLVVVTEHAIANFTKAEMALEFNREFLDKLRVLSVSPKYSDLLTYVDCYVGVSARQALNNFQKQVPVITPTRQTMYVDSIQAALKALEKWEIDLRVAQTLLPTNVPIGEVSCPMQSVVKLLDDQLPDDSLIRRYPKEAAVALAKRNGGIQWMDVSEGTVMNEAVNAVAASALAPSASSPPLEEKSKLTEQAMDLVTAAEPEIVASLVPVPAPVFAIPPKPADYNVRTLKIDEATWLRMIPKTMSTPFQIQVTDNTGTKWHLNLRGGTRVVNLDQIAPMRFVLDLGGKSYKETSWDPNGKKVGFIVFQSKIPFELWTAASQIGQATVVNYVQLYAEDSSFTAQSIIATTSLAYNYEPEQLNKTDPEVNYYLLATFIDSAAITPTNMTQPDVWDAMLTMSPLSAGEVTVKGAVVSEVVPAELIGSYTPESLNASLPNDAARCMIDRASKIAEAIKIDDDAGPDEYSPNSVPIQGQLAISQLETGYGVRIFNSKGILSKIASRAMQAFIGDPSTIITQAAPVLSDKNNWIALAQGVKTSLRTKSLSAGVKTAVSKLSSSESIQSWTQGFLDKVSMHFPAPKSDCPTSGDSSESSARRVKRDSYAGVVKRGYTR

SEQ ID NO: 17 - DNA, 2241 bp, Orthoreovirus
gctaaagtgaccgtggtcatggcttcgttcaagggattctccgccaacactgttccagtttccaaggccaaacgtgacatatcatcccttgctgctactcctggatttcattcacaatcctttactccgtctgtggatatgtctcaatcgcgtgaattcctcacaaaagcaatcgagcaggggtccatgtctataccttatcagcatgtgaatgtaccgaaagttgatcgtaaagttgtcagcttggtagtgcggcctttttcttcaggtgctttctctatctctggagtgatttcgccagcccatgcctatctgctagattgtctacctcagcttgagcaggcaatggcttttgttgcttcacccgagtctttccaggcttcagatgttgcaaagcgttttgctataaagccaggtatgagcctccaggacgctatcactgcgtttattaatttcgtgtccgcgatgctgaaaatgacggtgactcgtcagaattttgatgttattgtagctgagatcgagaggcttgcttcaaccagcgtgtctgtcaggactgaggaagcgaaggttgctgatgaggagctgatgttattcgggctagatcacagagggccacagcagttggatatttctgacgctaaagggataacgaaggctgctgacattcagacaactcatgatgttcatctggcacccggcgttggtaatattgaccctgaaatctataacgaagggcggttcatgttcatgcagcacaaaccacttgcggcggatcaatcgtactttaccttagagactgcggattatttcaagatttatccaacatatgacgaacatgatggtaggatggctgaccaaaagcagtcgggattgatactatgtactaaagatgaagtgttggctgagcaaactatatttaaactggacgctcccgacgacaaaactgttcatctgttagatcgtgacgacgaccacgttgttgccagatttaccaaggtatttatagaagacgtagctcccgggcatcacgctgctcagagatcgggacaacgctctgtgcttgatgacctatatgcgaatacgcaagtgatttccattacctccgccgctctgaagtgggtggttaaacatggcgtgtctgatggaattgtgaataggaagaatgtcaaagtgtgtgttggttttgaccctttatacactctgtccacgcataacggaatatctctgtgtgccctgttgatggatgagaagctttcggtgctgaacagtgcgtgtcgtatgacgttgcgctctctcatgaagaccggacgtgatgctgatgcacacagagcttttcagcgagtcctttctcaaggatacgcatcgttaatgtgctattatcacccttcacggaagctggcatatggcgaggtgcttcttccagaacggtccaatgacgtggtagatgggatcaagctacagttggacgcatccagacattgtcatgaatgtcctgtgttgcagcagaaagtggttgaattggaaaaacagatcgtcatgcaaaagtcgattcagtcagaccctaccccaatggcactgcaaccactgttgtctcagttgcgtgagctatccagcgaagttactaggctgcagatggagttgagtagggctcaatctttgaatgcccagttggaggcggatgtcaaatcagctcaatcatgcagcctggatatgtatctgagacaccacacttgcattaatggtcatgctaaagaggatgaattgcttgatgctgtgcgtgtcgcaccggatgtgaggaggcaaatcatggaaaggaggagtgaagtgagaaagggatggtgtgaacgtatttctaaggaagcgtctgccgaatgtcagaatgttattgatgatctgactctgatgaatggaaagcaggcccaagagataagagaattacgtgattcggctgagagttatgagaaacagattgcggagctggtgagtaccatcacccaaaaccagatgacttatcagcaagagttacaagccttagtagcgaaaaacgtggaattggatacattgaatcaacgtcaggctaggtcgttgcggattactccctctcttctatcagtcactcctaccgattcagttgatggcgctgctgacctaatcgatttctctgttccgactgatgagctgtaaatgatccgtgatgcagtgttgtcctaatcccttaagccttcccgacccccattcatc

SEQ ID NO: 18 - protein, 721 aa, Orthoreovirus
MASFKGFSANTVPVSKAKRDISSLAATPGFHSQSFTPSVDMSQSREFLTKAIEQGSMSIPYQHVNVPKVDRKVVSLVVRPFSSGAFSISGVISPAHAYLLDCLPQLEQAMAFVASPESFQASDVAKRFAIKPGMSLQDAITAFINFVSAMLKMTVTRQNFDVIVAEIERLASTSVSVRTEEAKVADEELMLFGLDHRGPQQLDISDAKGITKAADIQTTHDVHLAPGVGNIDPEIYNEGRFMFMQHKPLAADQSYFTLETADYFKIYPTYDEHDGRMADQKQSGLILCTKDEVLAEQTIFKLDAPDDKTVHLLDRDDDHVVARFTKVFIEDVAPGHHAAQRSGQRSVLDDLYANTQVISITSAALKWVVKHGVSDGIVNRKNVKVCVGFDPLYTLSTHNGISLCALLMDEKLSVLNSACRMTLRSLMKTGRDADAHRAFQRVLSQGYASLMCYYHPSRKLAYGEVLLPERSNDVVDGIKLQLDASRHCHECPVLQQKVVELEKQIVMQKSIQSDPTPMALQPLLSQLRELSSEVTRLQMELSRAQSLNAQLEADVKSAQSCSLDMYLRHHTCINGHAKEDELLDAVRVAPDVRRQIMERRSEVRKGWCERISKEASAECQNVIDDLTLMNGKQAQEIRELRDSAESYEKQIAELVSTITQNQMTYQQELQALVAKNVELDTLNQRQARSLRITPSLLSVTPTDSVDGAADLIDFSVPTDEL

SEQ ID NO: 19 - DNA, 1416 bp, Orthoreovirus
atgctattggtcggatggatcctcaactgcgtgaggaagtggtacgtctaataattgcgttgacaagcgataatggagcagtgttgtcaaaagaactcgggtcaagggtcacggcgcttgagaaaacgtcccagatacactctgatacaatccttaggatcactcaaggactcgaggatgcaaataaacgaatcagcgctcttgagcaaagtagggacggtttggttgcatcagttagtgatgcgcaacttgcaatctcccgattggaaggcgctgtcggagtcctccagacaactgtcaatggacttgattcgagtgttacccagttgggtggtagagtgggacagcttgagacaggatttgcaggattacgcaatgactacagcagtctctctacgcgaatgggtaatgtggaacgcgacactggatcattaacgactgaattggcgacgctcacgttacgtgttacttcgatccaatcagacttcgagtctagagtatcgacattagagcgtaccgcagttaccagtgctgccgcccctttggcaatcaataacaatcgtatgacgatggggctaaacgacggattgacactatcagggaataatcttgccatccggttgcctggtaacacgggattaagtattcaaaatggtgggcttcaatttcgatttaacactaatcaatttcagattgtcaataacagattaactcttaaaaccactgtttttgatcccctcaattcgagagtaagcacgatcgagcaaagctatgttgcgtctgcagtggcgcctttaaggttagatggcagcacgaaggtactggacatgttgatagatagctctacactcgagattaatgctaatgggcaactagctgtgaaatcaacttcgccgaacttaagatatccgattgctgatatcagtggtagtattgggatgagccctaactacagatttaggcgaagtatgtggataggacttatctcatactcgggtagtggactaagttggaggatacaggtcaattctgacgtctttatcgttgatgactacatacacatatgcctcccggcgtttaacggtttcacgatagctgacggtggcgatctgtcgttgaactttgttactggattactgccgccattactcactggcgatactgaacctgcatttcataacgacgtggtcacgtatggagcacggaccatttctattggattatcagcaggcggcacacctcaatacatcagcaagaatttgtgggtggagcaatggcaagatggtgtcctgagactgcgtgttgaagggggtgggatgatcacacattcgaatagtaaatggcctgccataacagtctcatatccacgtagcttcacgtgaggatcagaccaccccacggcactggggcacttaa

SEQ ID NO: 20 - protein, 455 aa, Orthoreovirus
MDPQLREEVVRLIIALTSDNGAVLSKELGSRVTALEKTSQIHSDTILRITQGLEDANKRISALEQSRDGLVASVSDAQLAISRLEGAVGVLQTTVNGLDSSVTQLGGRVGQLETGFAGLRNDYSSLSTRMGNVERDTGSLTTELATLTLRVTSIQSDFESRVSTLERTAVTSAAAPLAINNNRMTMGLNDGLTLSGNNLAIRLPGNTGLSIQNGGLQFRFNTNQFQIVNNRLTLKTTVFDPLNSRVSTIEQSYVASAVAPLRLDGSTKVLDMLIDSSTLEINANGQLAVKSTSPNLRYPIADISGSIGMSPNYRFRRSMWIGLISYSGSGLSWRIQVNSDVFIVDDYIHICLPAFNGFTIADGGDLSLNFVTGLLPPLLTGDTEPAFHNDVVTYGARTISIGLSAGGTPQYISKNLWVEQWQDGVLRLRVEGGGMITHSNSKWPAITVSYPRSFT

SEQ ID NO: 21 - DNA, 1331 bp, Orthoreovirus
gctattcgctggtcagttatggctcgcgctgcgttcctattcaagaccgttggatttggtggcctgcaaagtgtgccaattaatgatgagttgtcgtcacatctacttcgagccggtaattcgccatggcagctgacccagttcttagattggataagtcttggaagaggattagctacatcagctcttgttccaaccgctggttcaagatattaccagatgagttgtttactgagtggcactctccaaattccatttcgtcctaatcatcgatggggggatactaggtttctgcgtctagtgtggtcagctcctacgcttgacgggttggttgttgccccaccgcaggtcttagctcagccggcgttacaggctcaggcagatcgagtgtatgattgtgatgactacccattcttggctcgtgacccgagatttaagcatcgagtgtatcaacaattgagtgccgtgactctgctcaatttgacgggattcggtccaatttcctatgttcgagtagacgaagatatgtggagtggagatgtgaaccagcttcttatgaattacttcgggcatacgtttgcagaaattgcatacacattatgccaggcttcagccaatagaccttgggagcacgatggtacgtacgcgaggatgactcaaattatactgtccttattctggttatcgtatgttggtgtaattcatcaacagaatacttaccggacgttctatttccaatgcaatcggcgtggtgatgctgctgaagtatggattctttcctgttcattaaaccactccgcccagattagaccgggtaatcgcagtctatttgtcatgccaacaagtccagactggaatatggacgtcaatctaatcttaagttcaacgttgacagggtgcttgtgttcgagctctcagttaccgctaattgataataactcagtgcctgcggtttcgcggaacattcacggttggactggtagagctggtaaccagctccatggttttcaagtgcgacgaatggtgactgaattctgtgacagattgagacgcgatggggttatgactcaagctcagcaaaatcaagttgaagcgttggcaaatcaaactcaacagtttaagagggataagcttgaggcctgggctagggaagatgatcagtataatcaggctcatccgaattctccaatgttccgtacgaagccatttacgaatgcgcaatggggacgaggaaataccggagcgactagtgccgcaattgcagcccttatctaatcgtcttggagtgagggggtccccccacacccctcgcgactgaccacacattcatc

SEQ ID NO: 22 - protein, 418 aa, Orthoreovirus
MARAAFLFKTVGFGGLQSVPINDELSSHLLRAGNSPWQLTQFLDWISLGRGLATSALVPTAGSRYYQMSCLLSGTLQIPFRPNHRWGDTRFLRLVWSAPTLDGLVVAPPQVLAQPALQAQADRVYDCDDYPFLARDPRFKHRVYQQLSAVTLLNLTGFGPISYVRVDEDMWSGDVNQLLMNYFGHTFAEIAYTLCQASANRPWEHDGTYARMTQIILSLFWLSYVGVIHQQNTYRTFYFQCNRRGDAAEVWILSCSLNHSAQIRPGNRSLFVMPTSPDWNMDVNLILSSTLTGCLCSSSQLPLIDNNSVPAVSRNIHGWTGRAGNQLHGFQVRRMVTEFCDRLRRDGVMTQAQQNQVEALANQTQQFKRDKLEAWAREDDQYNQAHPNSPMFRTKPFTNAQWGRGNTGATSAAIAALI

SEQ ID NO: 23 - DNA, 1198 bp, Orthoreovirus
gctaaagtcacgcctgttgtcgtcactatggcttcctcactcagagctgcgatctctaagattaagagagatgatgctggtcagcaagtttgtcccaattatgtcatgctcaggtcatcggtcacaacgaaagtggtacgaaacgttgttgagtatcaaatccgtacaggtggattcttttcgtgcctagcaatgttgagaccgctccagtatgctaaacgtgaacgtctgcttggacaaaggaatctggaacgtatatcgactagggacattcttcagacacgcgatttgcactcattgtgcatgccaactcctgatgcgccaatgtccaatcatcaggcagccaccatgagagagttgatctgcagctatttcaaggtcgatcatgctgatgggttgaaatatatacccatggatgagagatattctccatcatcgcttgccagactgttcactatgggtatggctggcctacacattaccactgagccttcctacaaacgtgtgcccatcatgcacttggcggcagatttggactgcatgacgttagctttaccctacatgattacacttgatggtgacacggtggtacctgttgccccaacgctttctgcagaacagcttttggatgatggacttaaggggttagcatgcatggatatctcatacggatgtgaggtggacgctaacaaccgatcagctggtgaccagagcatggattcttcacgatgcatcaatgagttatattgcgaggaaacggcagaagctatctgtgtactcaaaacatgtcttgtgctgaactgtatgcaattcaaacttgagatggatgatttagcacacaacgctgctgagctggacaagatacagatgatgataccttttagtgaacgcgttttcagaatggcttctgcatttgctaccattgatgcccagtgtttcaggttttgtgtgatgatgaaggataagaatttgaagatagacatgcgtgaaacgatgagactttggactcgatcggcgctggatgattcagtggctacgtcatctctgagtgtttcgctggatcgaggtcgatgggtggcagctgatgctaatgatgctagattgctggtgtttccaattcgcgtgtaatgggtgagtgagccgatgtggtcgccaagacatgtgccggtgtcttggtggtgggtggcgcctaatcatc

SEQ ID NO: 24 - protein, 366 aa, Orthoreovirus
MASSLRAAISKIKRDDAGQQVCPNYVMLRSSVTTKVVRNVVEYQIRTGGFFSCLAMLRPLQYAKRERLLGQRNLERISTRDILQTRDLHSLCMPTPDAPMSNHQAATMRELICSYFKVDHADGLKYIPMDERYSPSSLARLFTMGMAGLHITTEPSYKRVPIMHLAADLDCMTLALPYMITLDGDTVVPVAPTLSAEQLLDDGLKGLACMDISYGCEVDANNRSAGDQSMDSSRCINELYCEETAEAICVLKTCLVLNCMQFKLEMDDLAHNAAELDKIQMMIPFSERVFRMASAFATIDAQCFRFCVMMKDKNLKIDMRETMRLWTRSALDDSVATSSLSVSLDRGRWVAADANDARLLVFPIRV

SEQ ID NO: 25 - DNA, 1196 bp, Orthoreovirus
gctatttttgcctcttcctagacgttgtcgcaatggaggtgtgtctacctaatggtcatcagatcgtcgactggattaacaatgcatttgaaggacgggtgtcgatttatagtgcacagcaaggatgggataagacaatctcagctcagcctgatatgatggtgtgtggtagcgctgttgtttgcatgcattgcttgggtgtggttggatcattacagcgaaagttgaaccatctgcctcatcataaatgtaatcagcaattgcgtgagcaggattatgttgacctacagtttgctgatcgtgtaaccgctcactggaaacgtggcatgttatcatttgtatctcagatgcatgctatcatgaacgatgtgacacctgaggagcttgaaagagtgagaactgatggtggcatcttggctgagctcaactggcttcaaatagagtctggatcaatgtttcgttcgattcactcaaactggactgacccccttcaggtggtcgaagacctagatactcagctagatcgctattggacagcattgaatttgatgattgattcatcggatctggtgccaaacttcatgatgcgtgacccatcgcatgcctttaatggagtgaagctggagggtgaagcgcgacagactcaattcccgcgcacattcgattccgggtcaaacttgaaatggggtgttatggtatatgattattctgaacttgaaggggattctcagaaaggacgatcttataggagagagatcgttactccagcgaaagactttggtcactttggtttatcccattattctcgcgcaacgacgccaatacttggcaagatgcctgctgtattttctggtatgttaaccgggaactgtaaaatgtatccgtttataaagggcactgctaagctgaaaacggttaagaagctagttgatgctgtgaactacacgtggagttttgagaagatcagatacgctttaggccctggtgggatgacgggatggtataatagaactatgcagcaagcgccaattgtgttgactcctgcggcactgactatgtttccggatatgaccagatttggtgatctacagtatccaatcacgattggcgatccggctgtccttgggtaaacgcctccatcttctcagcgccgggcctgaccaacctggtgtgacgtgggacaggctccattcatc

SEQ ID NO: 26 - protein, 365 aa, Orthoreovirus
MEVCLPNGHQIVDWINNAFEGRVSIYSAQQGWDKTISAQPDMMVCGSAVVCMHCLGVVGSLQRKLNHLPHHKCNQQLREQDYVDLQFADRVTAHWKRGMLSFVSQMHAIMNDVTPEELERVRTDGGILAELNWLQIESGSMFRSIHSNWTDPLQVVEDLDTQLDRYWTALNLMIDSSDLVPNFMMRDPSHAFNGVKLEGEARQTQFPRTFDSGSNLKWGVMVYDYSELEGDSQKGRSYRREIVTPAKDFGHFGLSHYSRATTPILGKMPAVFSGMLTGNCKMYPFIKGTAKLKTVKKLVDAVNYTWSFEKIRYALGPGGMTGWYNRTMQQAPIVLTPAALTMFPDMTRFGDLQYPITIGDPAVLG

SEQ ID NO: 27 - DNA, 3854 bp, Orthoreovirus
gctacacgttccacgacaatgtcatccatgatactgactcagtttggaccgttcattgaaagcatctcaggaatcactgaccaatcgaacgacgtgtttgaagatgcggcaaaagcgttctctacgtttactcgcagcgacgtctataaggcactggatgagatacctttctctgatgatgcaatgcttcccatccccccaactatatataccaaaccatctcacgattcatattattacatagatgctctaaaccgcgtacgtcgtaaaacatatcagggccctgatgacgtgtacgtacctaattgttccatcgttgaattgctagagccgcatgagactctgacatcttatgggcgtttgtctgaagcgattgagaatcgtgccaaggatggagacagccaagccagaattgcgacaacatacggtagaatcgctgagtctcaggctagacagattaaggctccattggagaagtttgtgttggcactattggtgtccgaagcggggggttctctatatgacccagttttgcagaagtatgatgagattccagatctatcgcataattgccctttatggtgttttagagaaatctgtcgtcacatatctggtccattaccagatcgagcaccttatctttacttatcggcaggggttttctggttaatgtcaccacggatgacgtctgcgatccctccgttattatctgatcttgttaatttagctatcttacaacagactgcgggtttagatccatcattagtgaaactgggagtgcagatatgccttcacgcggcagctagctcaagttatgcatggtttatcctaaagactaagtctatttttcctcaaaacacgttacatagtatgtatgagtctctagaaggagggtactgtcctaacctagaatggttagagcctagatcggactataaatttatgtacatgggagtcatgccattgtccactaaatatgctaggtcggcaccatccaacgaaaagaaagcgcgggaacttggtgagaaatatggattgagttcagttgtcagtgagcttcgtaaacggacaatggcttatgttaaacatgactttgcttcggtaaggtacattcgtgacgccatggcatgtactagcggcatttttctggtaagaacacccaccgagacggtattgcaagaatatacccaaagtccggagattaaggttcccatcccccacaaagactggacaggcccagtaggtgaaatcagaattctaaaagatacaaccagctccatcgcgcgctacttgtatagaacatggtacttagcagcggcaagaatggcggctcagccacgcacgtgggatccattgttccaggcgattatgagatctcaatacgtgacagctaggggtgggtctggcgcagcactccgcgaatctctgtatgcaattaatgtgtcgttacctgattttaagggcttaccagtgaaggcagcaactaagatatttcaggcggcacaattagcgaacctgccgttctcacacacatcagtggctatactagctgacacttcgatgggattgcgaaaccaggtgcagaggcgaccacgatccatcatgcccttaaatgtgccccaacagcaggtttcggcgccacatacattgaccgctgattatatcaattatcacatgaatctatcgactacgtctggtagcgcggtcattgagaaagtgattcctttaggtgtatacgcttcaagccctcctaaccaatcgattaacattgacatatctgcgtgcgacgcaagtattacttgggacttctttctatccgtgattatggcggctatacacgaaggtgtcgctagtagctccattggaaaaccgtttatgggggttcctgcatccattgtaaatgatgagtctgtcgttggagtgagagctgctaggccgatatcgggaatgcagaacatggttcagcatctatcaaaactgtacaaacgtggattttcatatagagtgaacgactctttttctccaggcaacgattttactcatatgactaccactttcccgtcaggttcaacagccacttctactgagcatactgccaataatagtacgatgatggaaactttcctgacagtatggggacccgaacatactgacgaccccgacgtcttacgtctaatgaagtctttgactattcaaaggaattacgtgtgtcaaggtgatgatggattgatgattatcgatgggaatactgctggtaaggtgaaaagtgaaactattcaaaagatgttagagttaatctcaaaatatggtgaggagtttggatggaaatatgacatagcgtacgatgggactgccgagtacctaaagctgtacttcatatttggctgtcgaattccaaatcttagccgtcatccaattgttggaaaagaacgggcgaattcttcagcagaggagccatggccagcaattttagatcagattatgggtatcttctttaatggcgttcatgacgggttgcagtggcagcggtggatacgttattcatgggctctatgctgtgctttctcacgccaaaggacaatgattggcgagagcgtgggttacattcaatatcctatgtggtcatttgtctactggggattaccattggtaaaagtgttcgggtcagacccatggatattctcttggtacatgccgactggggacttgggaatgtatagttggattagcctaatacgccctctaatgacaagatggatggtagctaatggctatgtcactgacaaatgctcacccgtattcgggaacgcagattatcgtaaatgtttcaatgagattaaattatatcaagggtattatatggcacaattgcccaggaatcccacaaaatctggacggacggcccctcgggaggtaagagagcagttcactcaggcactctctgattatctgatgcagaatccagaactgaagtcacgcgtgctgcgtggtcgtagtgagtgggagaagtatggagcggggataattcacaatcctccatcattattcgatgtcccccataagtggtatcagggtgcgcaagaggcggcgaccgctacgagagaagagctggcagaaatggatgagacgttgatacgcgcccgaaggcacagttattcgagtttctcaaaattgttggaggcatacctgcttgtgaaatggcgaatgtgcgaggcccgcgaaccgtcggttgatttgcgattaccattgtgtgcgggtattgacccactaaactcagatccttttctcaaaatggtaagcgttggaccgatgcttcagagtacgcgaaagtactttgctcagacactattcatggcgaaaacggtgtcgggtctcgacgttaacgcgattgatagcgcgttattacgactgcgaacattgggcgctgataagaaagcattaacagcgcagttattaatggtgggacttcaggagtcagaggcggatgcgttggctgggaagataatgttgcaagatgtaagtactgtgcaattagctagagtggtcaatttagcggtgccagatacttggatgtcgttagattttgattctatgttcaaacaccatgtcaaactgcttcccaaagatggacgccacctaaatactgatattcctcctcgcatgggatggttacgggccattctacgattcttaggtgctggaatggtaatgactgcgactggagttgctgtcgacatatatctggaggatatacacggtggtggtcgatcacttggacagagattcatgacttggatgcggcaggaaggacggtcagcgtgagtctaccatgggtcgtggtgcgtcaactcatc

SEQ ID NO: 28 - protein, 1267 aa, Orthoreovirus
MSSMILTQFGPFIESISGITDQSNDVFEDAAKAFSTFTRSDVYKALDEIPFSDDAMLPIPPTIYTKPSHDSYYYIDALNRVRRKTYQGPDDVYVPNCSIVELLEPHETLTSYGRLSEAIENRAKDGDSQARIATTYGRIAESQARQIKAPLEKFVLALLVSEAGGSLYDPVLQKYDEIPDLSHNCPLWCFREICRHISGPLPDRAPYLYLSAGVFWLMSPRMTSAIPPLLSDLVNLAILQQTAGLDPSLVKLGVQICLHAAASSSYAWFILKTKSIFPQNTLHSMYESLEGGYCPNLEWLEPRSDYKFMYMGVMPLSTKYARSAPSNEKKARELGEKYGLSSVVSELRKRTMAYVKHDFASVRYIRDAMACTSGIFLVRTPTETVLQEYTQSPEIKVPIPHKDWTGPVGEIRILKDTTSSIARYLYRTWYLAAARMAAQPRTWDPLFQAIMRSQYVTARGGSGAALRESLYAINVSLPDFKGLPVKAATKIFQAAQLANLPFSHTSVAILADTSMGLRNQVQRRPRSIMPLNVPQQQVSAPHTLTADYINYHMNLSTTSGSAVIEKVIPLGVYASSPPNQSINIDISACDASITWDFFLSVIMAAIHEGVASSSIGKPFMGVPASIVNDESVVGVRAARPISGMQNMVQHLSKLYKRGFSYRVNDSFSPGNDFTHMTTTFPSGSTATSTEHTANNSTMMETFLTVWGPEHTDDPDVLRLMKSLTIQRNYVCQGDDGLMIIDGNTAGKVKSETIQKMLELISKYGEEFGWKYDIAYDGTAEYLKLYFIFGCRIPNLSRHPIVGKERANSSAEEPWPAILDQIMGIFFNGVHDGLQWQRWIRYSWALCCAFSRQRTMIGESVGYIQYPMWSFVYWGLPLVKVFGSDPWIFSWYMPTGDLGMYSWISLIRPLMTRWMVANGYVTDKCSPVFGNADYRKCFNEIKLYQGYYMAQLPRNPTKSGRTAPREVREQFTQALSDYLMQNPELKSRVLRGRSEWEKYGAGIIHNPPSLFDVPHKWYQGAQEAATATREELAEMDETLIRARRHSYSSFSKLLEAYLLVKWRMCEAREPSVDLRLPLCAGIDPLNSDPFLKMVSVGPMLQSTRKYFAQTLFMAKTVSGLDVNAIDSALLRLRTLGADKKALTAQLLMVGLQESEADALAGKIMLQDVSTVQLARVVNLAVPDTWMSLDFDSMFKHHVKLLPKDGRHLNTDIPPRMGWLRAILRFLGAGMVMTATGVAVDIYLEDIHGGGRSLGQRFMTWMRQEGRSA

SEQ ID NO: 29 - DNA, 3915 bp, Orthoreovirus
gctattggcgcaatggcgaacgtttggggagtgagacttgcagactctttatcgtcacccactattgagacaagaactcgtcattacacactccgcgatttctgttccgacctggatgctgtagttggcaaggaaccctggagacccttacgcaatcagagaacgaatgatattgtcgccgttcaattgtttcggccactgcagggattggtgcttgacacgcagttttatggattccctggcattttctcagaatgggaacagtttataagagagaaactacgcgtgttgaaatatgaagttttgcggatttacccgatcagtaattataatcatgagcgtgtcaatgtcttcgtggcaaatgctcttgtcggtgcatttctatccaaccaagccttctatgacctgttgcctctactattaatacgtgataccatgataaatgacttacttgggacaggtgctgctctttctcagtttttccaatctcatggtgaggttttagaggttgccgcaggaaggaagtacctgcaaatgaagaactactcgaacgatgatgatgatccacctttattcgctaaggatctgtcggattatgcgaaggcgttttacagtgatacgtttgagactttagaccgattcttctggacacatgactcatctgcgggcgtcctagtgcattatgataagcctaccaatgggaatcattacatcttgggtactctgacgcagatggttagtgcgcctccgcatatcattaacgctactgacgcattgttgctcgaatcgtgtttagaacaatttgcggagaatgtgagagccaggccagcgcagcctgttccaagattggatcagtgttaccatttacggtggggtgctcaatatgttggcgaggactcattgacgtaccgtttgggggtactttcactactggctaccaacggatatcaattagctagaccgatccctaagcagttaacgaatcgatggctttctagttttgtcagtcagataatgtcggatggtgtgaatgagacgccattatggcctcaagagagatatgtccaaatagcctacgattcaccgtctgtagtcgacggagctacgcactatggttatgttaggagaaatcagttgcggttgggcatgagggtgtccgctcttcagtcattgagtgatactccggctccgatacagtggttaccgcagtatactattgatcaggcacctgttgatgagggagatctaatggtttcgcggttgactcaactaccgttacgccctgattatggtagcatatgggtcggtgacgctctatcgtattatgttgattacaaccgcagccatagagttgtactatcatccgagctaccacaactaccagatacatactttgacggagacgagcaatacggtcgcagtctgttctctttagcacgaaaaatcggtgatcgatctctcatcaaagatacagcagtgctcaagcatgcgtaccaggccatcgatccaaacactggaaaggaataccttcgcgcaggacagtctgttgcatatttcggagcatcagctggtcattcaggggcggatcaacctctagtaattgagccatggacgcagggtaaaattagtggtgtaccgcagccttcttcagtcagacagtttgggtatgatgttgctaaaggtgcgattgtggacttagcaagaccgttcccgtcgggtgactaccaatttgtatattctgacgtcgatcaggtcgttgacggccacgatgatctcagcatatcttcagggctggtggagagtctattagattcctgcatgcatgccacatccccaggtgggtcgttcgtgatgaagataaatttcccgacacgtgatgtctggcactatatagagcaaaagattctcccaaatattacctcgtacatgttgatcaaaccattcgtgactaacaatgtagagttattctttgtggctttcggtgtgcatcaacaatcagcattgacatggacgtccggggtgtatttcttcctggtcgatcacttctatcgatacgagacattgtctacgatttcacgtcagttgccatcgttcggatacgttgatgacgggtcgtctgtgacaggtattgagatgatcagtcttgaaaatccaggcttttcaaacatgacccaagctgcacgtgtcgggatatcagggctgtgtgcgaatgtcggtaatgcgcgcaaattaatatctatccatgaatctcacggagcacgcgtgctcaccatcatatcgagaagatctccggcttcggctaggcggaaagctcgcttacgctatttgccactcatagacccacgatctttggaagtgcaggcacgtacgatattaccatctaacccagtgctgtttgacaacgtaaaaggagcatcgcctcacgtatgtttgacgatgatgtataactttgaagtatctagtgcggtgtatgatggtgatgtagtgcttgaccttggtaccggtcctgaagcgaagattctggagctgattcctccaacgtccccagtaacatgcgtggacattagaccgacggcacagcctagtggctgttggaacgtacgtacgacatttctggagcttgattacctaagtgatggctggataacgggtgtacgtggcgacatcgtgacctgcatgctgtccctgggtgctgctgctgctggaaaatccatgacgttcgacgcggcatttcaacagttagtgaaagtgcttactaaaagtacagctaacgtactgctgatccaagtcaactgcccaacggatgtaatccgaacaattaagggatatttggagatagatcaaactaataagcggtatagatttcccaaatttggccgtgatgaaccatactctgacatggattccttagagcgcatatgtcgtgctgcgtggccaaattgttccatcacgtgggtgcctttatcctatgatctacgttggactaaacttgctttgcttgaatcgactacactgagcagtgcatcagtgagaattgctgagttgatgtacaaatacatgccagttatgaggatagatattcatgggttacccatggaaaagcaaggcaatttcgtagtgggtcagaactgttctctaactataccgggcttcaacgcacaggacgtgttcaactgctacttcaattccgcgctcgctttctctactgaggatgttaattcggcaatgataccacaagtgacggctcagtttaacactagtaaaggtgagtggtcattggacatggtgttctcagacgctggtatctacacaatgcaggcattagtaggttccaacgcaaatcctgtgtctttgggttcgtttgtagtggattctccggatgtcgacataacagatgcgtggcctgctcagttagattttaccatagctggcactgatgtcaacatcacagttaatccttattaccgcttgatggcctttgtaaagattgatggacaatggcagattgcgaaccctgataaattccaatttttctcatcaggtacagggacgttagtgatgaatgtaaagttagatatagctgataggtatttgctatattacattcgcgacgttcaatctagggatgtgggattttacatacagcacccattacagttattaaatacaattacgttgcctacaaacgaggatttattcttgagcgctcctgacatgcgcgagtgggcggtaaaggaaagtggcaataccatatgcatacttaatagccagggttttgtgccacctcaggattgggatgttcttaccgacactattagctggtctccttcgctcccaacttatgtggtacctccgggtgattatactctgacacctctgtaactcattacccctcgtaagcgtgcctaattcatc

SEQ ID NO: 30 - protein, 1289 aa, Orthoreovirus; identical to SEQ ID NO: 10

SEQ ID NO: 31 - DNA, 3901 bp, Orthoreovirus
gctaatcgtcaggatgaagcggattccaaggaagacaaagggcaaatccagcggaaagggcaatgactcaatagatagagcggacgatggctcaagccaattacgagacaagcaaaataataagaccggccccgccactacagagcctgggacatccaaccgagagcagtacaaagctcgaccaagtattgcatctgtgcagagggccactgaaagtgcagaactacctatgaagaacaatgacgaaggaacgccagataagaagggaaatactaagggcgacttagtcagtgaacatggtgaggctaaagacgaggcggatgaagcgacgaagaagcaggcaaaagatactgatagaagtaaggcgcaagttacatattcagacactggtatcaataatgctaatgaactgtcaagatctgggaatgtggataatgagggtggaagtaatcagaagccgatgtccaccagaatagctgaagcaacgtcggctatagtgtcgaaacatcctgcgcgtgttgggttaccacctaccgctagcagtggtcatgggtatcagtgtcatgtctgttctgcagtcctgtttagtcctttagacctagacgcccacgtcgcctcacatggtttgcatggtaatatgacattgacatcgagtgagatccagcgacatatcactgagtttatcagttcatggcaaaatcatcctattgttcaagtttcggctgacgtcgaaaataagaagactgctcaattgctgcacgctgacactcctcgacttgtcacttgggatgctggtctgtgtacctcgtttaaaatcgtcccgattgtgccagctcaggtaccgcaggatgtattggcctatacgttctttacctcttcatacgctattcaatcaccgtttccagaggcggcagtgtctaggattgtggtgcatacaagatgggcatctaatgttgacttcgaccgagattcgtctgtcatcatggcaccacctacagaaaataatatccatttgtttaagcagttgctaaacactgataccctgtctgtgagaggggccaacccgctaatgtttagagcgaatgtattgcatatgttgctggagttcgtattggataacttgtatttgaacagacatacgggattctctcaagatcacacaccatttactgagggcgctaatctgcgttcacttcctggccccgatgctgagagatggtattcgattatgtatccaacgcgtatgggaacgccgaacgtatcgaagatatgtaatttcgtcgcctcttgtgtgcgaaatcgagtcggaaggtttgatcgagcacagatgatgaacggagccatgtcagagtgggtggatgtcttcgagacttcagacgcgcttaccgtttccattcgaggccgatggatggctagattagctcgcatgaacataaatccaacagagatcgagtgggcgttgactgaatgtgcacaaggatatgtgactgttacaagtccttacgctcctagcgtaaatagattgatgccctatcgtgtctctaacgctgagcggcagatatcacagataatcaggatcatgaacatcggcaataacgcgacggtgatacagcctgttctgcaagatatttcagtgctccttcaacgcatatcaccactccaaatagatccaaccattatttccaacactatgtcaacagtttcggagtctactactcagacactcagccccgcgtcctcaattttgggtaaattacgaccgagtaactcagatttctctagttttagagtcgcgttggctggatggctttataatggagttgtgacgacggtgattgatgatagttcatatccaaaggacgggggcagcgtgacctcacttgaaaatctgtgggattttttcatccttgcgcttgctttaccactgacaactgacccatgtgcacctgtgaaagcgtttatgactttagccaacatgatggttggtttcgagacaatccccatggataatcagatctatactcaatcgagacgtgcgagtgctttctcaacgcctcatacgtggccacgatgcttcatgaacatccagttaatttctcccatcgacgctcccatcttacgacagtgggctgaaattattcatagatactggcctaatccttcacagatccgttatggtgcaccgaacgtttttggttcggcaaatctgttcactccacctgaggtgctgttattgccaatcgatcatcaaccagctaatgtgacaacgccgacgctggacttcaccaacgagttgaccaattggcgcgctcgtgtctgtgagcttatgaagaatcttgttgataatcaaagatatcaacctggatggacacaaagtctagtttcgtcaatgcgcggaacgctggacaaattgaaattgatcaaatcgatgacaccaatgtatctgcaacagctggctccggtagagttagcggtaatagctcccatgttgccttttccacctttccaggtgccttacgttcgacttgatcgtgacagagttccaacaatggtcggagtaacacgacagtcacgagatactattactcagccagcgctgtcattgtcgacaaccaataccactgttggtgtgcctttagctctggacgcaagggctattactgttgcgctgttgtcagggaaatatccgccggatctagtgacaaatgtatggtacgctgacgccatttatccaatgtatgcagatactgaggtgttctctaatcttcagagagacatgattacttgcgaagccgtgcagacgttagtgactctggtggcgcagatatcagagacccagtatcctgtagataggtatcttgattggatcccatcgctgagagcatcggcagcgacggcagcaacgtttgctgagtgggttaatacttcaatgaagacggcgtttgatttgtctgacatgctgttagagcctctactgagcggggatccgaggatgactcaactagcgattcagtatcagcaatacaatggcagaacgtttaatgtcatacctgaaatgccaggctcggtcatagctgactgtgttcaactaacagcagaagtcttcaatcacgaatataacctgtttggaattgcgaggggtgatatcatcattggccgtgtccagtcgacacacttgtggtcaccactggctcctccacctgatctggtgtttgatcgtgatactcctggcgttcacatcttcggacgagattgccgtatatcgtttggaatgaatggcgccgcgccaatgattagagatgagactggaatgatggtgcctttcgaaggaaattggattttcccactggcgctttggcaaatgaatacgcgatattttaatcaacagttcgatgcgtggattaagacaggagagttgcgaatccgtattgagatgggtgcgtacccatatatgttgcattactatgatccacgtcagtacgctaatgcatggaatttgacatccgcctggcttgaggaaattacaccgacgagcattccatccgtgcctttcatggtgccaatctcaagtgatcatgatatttcctctgccccagctgtccaatatatcatttcgactgaatataatgatcggtctctattctgcactaattcatcatctccccaaaccatcgctggaccagacaaacacattccagttgaacgatataacattctgaccaaccccgatgctccacccacgcagatacaactgcctgaagttattgatttgtataatgtcgtcacacgctatgcgtatgagactccacctattaccgctgttgttatgggcgttccttgatcctcatcctcccaacaggtgctagagcatcgcgctcgatgctagttgggccgattcatc

SEQ ID NO: 32 - protein, 1275 aa, Orthoreovirus
MKRIPRKTKGKSSGKGNDSIDRADDGSSQLRDKQNNKTGPATTEPGTSNREQYKARPSIASVQRATESAELPMKNNDEGTPDKKGNTKGDLVSEHGEAKDEADEATKKQAKDTDRSKAQVTYSDTGINNANELSRSGNVDNEGGSNQKPMSTRIAEATSAIVSKHPARVGLPPTASSGHGYQCHVCSAVLFSPLDLDAHVASHGLHGNMTLTSSEIQRHITEFISSWQNHPIVQVSADVENKKTAQLLHADTPRLVTWDAGLCTSFKIVPIVPAQVPQDVLAYTFFTSSYAIQSPFPEAAVSRIVVHTRWASNVDFDRDSSVIMAPPTENNIHLFKQLLNTDTLSVRGANPLMFRANVLHMLLEFVLDNLYLNRHTGFSQDHTPFTEGANLRSLPGPDAERWYSIMYPTRMGTPNVSKICNFVASCVRNRVGRFDRAQMMNGAMSEWVDVFETSDALTVSIRGRWMARLARMNINPTEIEWALTECAQGYVTVTSPYAPSVNRLMPYRVSNAERQISQIIRIMNIGNNATVIQPVLQDISVLLQRISPLQIDPTIISNTMSTVSESTTQTLSPASSILGKLRPSNSDFSSFRVALAGWLYNGVVTTVIDDSSYPKDGGSVTSLENLWDFFILALALPLTTDPCAPVKAFMTLANMMVGFETIPMDNQIYTQSRRASAFSTPHTWPRCFMNIQLISPIDAPILRQWAEIIHRYWPNPSQIRYGAPNVFGSANLFTPPEVLLLPIDHQPANVTTPTLDFTNELTNWRARVCELMKNLVDNQRYQPGWTQSLVSSMRGTLDKLKLIKSMTPMYLQQLAPVELAVIAPMLPFPPFQVPYVRLDRDRVPTMVGVTRQSRDTITQPALSLSTTNTTVGVPLALDARAITVALLSGKYPPDLVTNVWYADAIYPMYADTEVFSNLQRDMITCEAVQTLVTLVAQISETQYPVDRYLDWIPSLRASAATAATFAEWVNTSMKTAFDLSDMLLEPLLSGDPRMTQLAIQYQQYNGRTFNVIPEMPGSVIADCVQLTAEVFNHEYNLFGIARGDIIIGRVQSTHLWSPLAPPPDLVFDRDTPGVHIFGRDCRISFGMNGAAPMIRDETGMMVPFEGNWIFPLALWQMNTRYFNQQFDAWIKTGELRIRIEMGAYPYMLHYYDPRQYANAWNLTSAWLEEITPTSIPSVPFMVPISSDHDISSAPAVQYIISTEYNDRSLFCTNSSSPQTIAGPDKHIPVERYNILTNPDAPPTQIQLPEVIDLYNVVTRYAYETPPITAVVMGVP

SEQ ID NO: 33 - DNA, 2304 bp, Orthoreovirus
gctcttcgcggtcatggcttacatcgcagttcctgcggtggtggattcacgttcgagtgaggctattggactactagaatcgtttggagtagacgctggggctgatgtgaatgatgtttcatatcaagatcatgactatgtgttggatcagttacagtatatgttagatgggtatgaggctggtgacgtcatcgatgcactcgtccacaagaattggttacatcattctgtctattgcttgttgccacccaaaagtcaactactagagtattggaaaagtaacccttcagcgataccggacaacgttgatcgtcggcttcgtaaacggctaatgctaaagaaagatctcagaaaagatgatgagtacaatcaattggcgcgtgctttcaagatatcggatgtctacgcaccactcatctcatctacgacgtcaccgatgacaatgatccagaacttgaatcagggcgagatcgtgtacaccacgacggacagagtaattggggctagaatcttgttatatgctccaagaaagtactatgcatcaactctatcatttactatgactaagtgcatcattccgtttggcaaagaggtgggccgtgctcctcactctagatttaatgttggcacattcccatcaattgctactccgaagtgttttgttatgagtggggttgatattgagtccatcccaaatgaatttatcaaattgttttaccagcgcgtcaagagtgttcacgctaatatactaaatgacatatcacctcagatactctctgacatgataaacagaaagcgtttgcgtgttcatactccatcagatcgtcgagccgcgcaactgatgcatttgccctatcatgttaagcgaggggcgtctcacgtcgacgtttataaggtagatgttgtggatgtattgtttgaggtagtagatgtggccgatgggttgcgcaatgtatctaggaagctaactatgcacactgttccggtctgtattcttgaaatgttgggtattgagattgcggactattgcgttcgtcgagaggatggaatgttcacagattggttcttgcttttaaccatgctatctgatggcttaactgatagaaggacgcgttgtcaatacctgattaatccgtcaagcgtgcctcctgatgtaatacttaacatctctattactggatttataaacaggcatacaatcgacgtcatgcctgacacatacgacttcattaaacccattggtgctgtgctgcctaagggatcattcaaatcgacaattatgagagttcttgactcaatatcaatattaggagttcagatcatgccgcgcacgcatgtagtcgactcggatgaggtgggcgagcaaatggagcctacgtttgagcatgcggtcatggagatatacagaggaattgctggcgttgactctctggatgatctcattaggtgggtgctgaactcggatctcattccatatgatgacaggcttggccaattatttcaagcgtttctgcctctcgcaaaagatttgttagcgccaatggccagaaagttttatgataactcaatgagtgagggtagattgctgacattcgctcatgctgatagtgagttgctgaacgcaaattactttggtcatttactgcgactaaaaataccatatattacagaggttaatttgatgattcgcaagaatcgtgagggtggggagctatttcagcttgtgttatcacatctatataaaatgtatgctactagcgcgcagcctaaatggtttggatcattattgcgattgttaatatgtccctggttacatatggagaaattgataggagaagcagacccagcatctacgtcggctgaaattggatggtatatctctcgtgaacagctgatgcaagatggatggtgtggatgtgaagatggattcattccctatattagcatacgtgcgccaaagctggttatagaggagttaatggagaagaattggggccaatatcatgcacaagttattatcactgatcggcttgtcgtaggcgaaccgcgtagggtatctgccaaggctgtggtcaaaggtaaccacttaccagttaagttagtctcacgatttgcatgtttcacactgacgacgaagtatgagatgaggctttcatgtggccatagcactggacggggggctgcatacaatgcgagactagttttccgatctgacttggcgtgatccgtgacatgcgtagtgtgacacctgcccctaggtcaatgggggtagggggcgggctaggactacgtacgcgcttcatc

SEQ ID NO: 34 - protein, 736 aa, Orthoreovirus; identical to SEQ ID NO: 14

SEQ ID NO: 35 - DNA, 2205 bp, Orthoreovirus; identical to SEQ ID NO: 15

SEQ ID NO: 36 - protein, 708 aa, Orthoreovirus; identical to SEQ ID NO: 16

SEQ ID NO: 37 - DNA, 2241 bp, Orthoreovirus; identical to SEQ ID NO: 17

SEQ ID NO: 38 - protein, 721 aa, Orthoreovirus; identical to SEQ ID NO: 18

SEQ ID NO: 39 - DNA, 1416 bp, Orthoreovirus
atgctattggtcggatggatcctcaactgcgtgaggaagtggtacgtctaataattgcgttgacaagcgataatggagcagtgttgtcaaaagaactcgggtcaagggtcacggcgcttgagaaaacgtcccagatacactctgatacaatccttaggatcactcaaggactcgaggatgcaaataaacgaatcagcgctcttgagcaaagtagggacggtttggttgcatcagttagtgatgcgcaacttgcaatctcccgattggaaggcgctgtcggagtcctccagacaactgtcaatggacttgattcgagtgttacccagttgggtggtagagtgggacagcttgagacaggatttgcaggattacgcaatgactacagcagtctctctacgcgaatgggtaatgtggaacgcgacactggatcattaacgactgaattggcgacgctcacgttacgtgttacttcgatccaatcagacttcgagtctagagtatcgacattagagcgtaccgcagttaccagtgctgccgcccctttggcaatcaataacaatcgtatgacgatggggctaaacgacggattgacactatcagggaataatcttgccatccggttgcctggtaacacgggattaagtattcaaaatggtgggcttcaatttcgatttaacactaatcaatttcagattgtcaataacggattaactcttaaaaccactgtttttgatcccctcaattcgagagtaagcacgatcgagcaaagctatgttgcgtctgcagtggcgcctttaaggttagatggcagcacgaaggtactggacatgttgatagatagctctacactcgagattaatgctaatgggcaactagctgtgaaatcaacttcgccgaacttaagatatccgattgctgatatcagtggtagtattgggatgagccctaactacagatttaggcgaagtatgtggataggacttatctcatactcgggtagtggactaagttggaggatacaggtcaattctgacgtctttatcgttgatgactacatacacatatgcctcccggcgtttaacggtttcacgatagctgacggtggcgatctgtcgttgaactttgttactggattactgccgccattactcactggcgatactgaacctgcatttcataacgacgtggtcacgtatggagcacggaccatttctattggattatcagcaggcggcacacctcaatacatcagcaagaatttgtgggtggagcaatggcaagatggtgtcctgagactgcgtgttgaagggggtgggatgatcacacattcgaatagtaaatggcctgccataacagtctcatatccacgtagcttcacgtgaggatcagaccaccccacggcactggggcacttaa

SEQ ID NO: 40 - protein, 455 aa, Orthoreovirus
MDPQLREEVVRLIIALTSDNGAVLSKELGSRVTALEKTSQIHSDTILRITQGLEDANKRISALEQSRDGLVASVSDAQLAISRLEGAVGVLQTTVNGLDSSVTQLGGRVGQLETGFAGLRNDYSSLSTRMGNVERDTGSLTTELATLTLRVTSIQSDFESRVSTLERTAVTSAAAPLAINNNRMTMGLNDGLTLSGNNLAIRLPGNTGLSIQNGGLQFRFNTNQFQIVNNGLTLKTTVFDPLNSRVSTIEQSYVASAVAPLRLDGSTKVLDMLIDSSTLEINANGQLAVKSTSPNLRYPIADISGSIGMSPNYRFRRSMWIGLISYSGSGLSWRIQVNSDVFIVDDYIHICLPAFNGFTIADGGDLSLNFVTGLLPPLLTGDTEPAFHNDVVTYGARTISIGLSAGGTPQYISKNLWVEQWQDGVLRLRVEGGGMITHSNSKWPAITVSYPRSFT

SEQ ID NO: 41 - DNA, 1331 bp, Orthoreovirus
gctattcgctggtcagttatggctcgcgctgcgttcctattcaagaccgttggatttggtggcctgcaaagtgtgccaattaatgatgagttgtcgtcacatctacttcgagccggtaattcgccatggcagctgacccagttcttagattggataagtcttggaagaggattagctacatcagctcttgttccaaccgctggttcaagatattaccagatgagttgtttactgagtggcactctccaaattccatttcgtcctaatcatcgatggggggatactaggtttctgcgtctagtgtggtcagctcctacgcttgacgggttggttgttgccccaccgcaggtcttagctcagccggcgttacaggctcaggcagatcgagtgtatgattgtgatgactacccattcttggctcgtgacccgagatttaagcatcgagtgtatcaacaattgagtgccgtgactctgctcaatttgacgggattcggtccaatttcctatgttcgagtagacgaagatatgtggagtggagatgtgaaccagcttcttatgaattacttcgggcatacgtttgcagaaattgcatacacattatgccaggcttcagccaatagaccttgggagcacgatggtacgtacgcgaggatgactcaaattatactgtccttattctggttatcgtatgttggtgtaattcatcaacagaatacttaccggacgttctatttccaatgcaatcggcgtggtgatgctgctgaagtatggattctttcctgttcattaaaccactccgcccagattagaccgggtaatcgcagtctatttgtcatgccaacaagtccagactggaatatggacgtcaatctaatcttaagttcaacgttgacagggtgcttgtgttcgagctctcagttaccgctaattgataataactcagtgcctgcggtttcgcggaacattcacggttggactggtagagctggtaaccagctccatggttttcaagtgcgacgaatggtgactgaattctgtgacagattgagacgcgatggggttatgactcaagctcagcaaaatcaagttgaagcgttggcagatcaaactcaacagtttaagagggataagcttgaggcctgggctagggaagatgatcagtataatcaggctcatccgaattctccaatgttccgtacgaagccatttacgaatgcgcaatggggacgaggaaataccggagcgactagtgccgcaattgcagcccttatctaatcgtcttggagtgagggggtccccccacacccctcgcgactgaccacacattcatc

SEQ ID NO: 42 - protein, 418 aa, Orthoreovirus
MARAAFLFKTVGFGGLQSVPINDELSSHLLRAGNSPWQLTQFLDWISLGRGLATSALVPTAGSRYYQMSCLLSGTLQIPFRPNHRWGDTRFLRLVWSAPTLDGLVVAPPQVLAQPALQAQADRVYDCDDYPFLARDPRFKHRVYQQLSAVTLLNLTGFGPISYVRVDEDMWSGDVNQLLMNYFGHTFAEIAYTLCQASANRPWEHDGTYARMTQIILSLFWLSYVGVIHQQNTYRTFYFQCNRRGDAAEVWILSCSLNHSAQIRPGNRSLFVMPTSPDWNMDVNLILSSTLTGCLCSSSQLPLIDNNSVPAVSRNIHGWTGRAGNQLHGFQVRRMVTEFCDRLRRDGVMTQAQQNQVEALADQTQQFKRDKLEAWAREDDQYNQAHPNSPMFRTKPFTNAQWGRGNTGATSAAIAALI

SEQ ID NO: 43 - DNA, 1198 bp, Orthoreovirus
gctaaagtcacgcctgttgtcgtcactatggcttcctcactcagagctgcgatctctaagattaagagagatgatgctggtcagcaagtttgtcccaattatgtcatgctcaggtcatcggtcacaacgaaagtggtacgaaacgttgttgagtatcaaatccgtacaggtggattcttttcgtgcctagcaatgttgagaccgctccagtatgctaaacgtgaacgtctgcttggacaaaggaatctggaacgtatatcgactagggacattcttcagacacgcgatttgcactcattgtgcatgccaactcctgatgcgccaatgtccaatcatcaggcagccaccatgagagagttgatctgcagctatttcaaggtcgatcatgctgatgggttgaaatatatacccatggatgagagatattctccatcatcacttgccagactgtttaccatgggtatggctggcctccacattaccactgagccttcctacaaacgtgtgcccatcatgcacttagcggcagatttggactgcatgacgttggctctaccctatatgattacacttgatggtgacacggtggtacctgttgccccgacgctttctgcagaacagcttttggatgatggacttaaggggttagcctgcatggatatctcatacggatgtgaggtggacgctaataaccgatcagctggtgaccagagcatggattcttcacgatgcatcaatgagttatattgcgaggaaacggcagaagctatctgcgtactcaaaacatgtcttgtgctgaactgtatgcaattcaaacttgagatggatgatttagcacacaatgctgctgagctggacaagatacagatgatgataccttttagtgaacgcgtgttcagaatggcttctgcatttgctaccattgacgcccagtgtttcaggttctgtgtgatgatgaaggataagaatttgaagatagatatgcgtgaaacgatgagactttggactcgatcggcgctggatgattcagtggctacgtcgtctttgagtatttcgctggatcgaggtcgatgggtggcagctgatgctaatgatgctaggttgctggtgtttccaattcgcgtgtaatgggtgagtaagccgatgtggtcgccaaggcatgtgccggtgtcttggtggtgggtggcgcctaatcatc

SEQ ID NO: 44 - protein, 366 aa, Orthoreovirus
MASSLRAAISKIKRDDAGQQVCPNYVMLRSSVTTKVVRNVVEYQIRTGGFFSCLAMLRPLQYAKRERLLGQRNLERISTRDILQTRDLHSLCMPTPDAPMSNHQAATMRELICSYFKVDHADGLKYIPMDERYSPSSLARLFTMGMAGLHITTEPSYKRVPIMHLAADLDCMTLALPYMITLDGDTVVPVAPTLSAEQLLDDGLKGLACMDISYGCEVDANNRSAGDQSMDSSRCINELYCEETAEAICVLKTCLVLNCMQFKLEMDDLAHNAAELDKIQMMIPFSERVFRMASAFATIDAQCFRFCVMMKDKNLKIDMRETMRLWTRSALDDSVATSSLSISLDRGRWVAADANDARLLVFPIRV

SEQ ID NO: 45 - DNA, 1195 bp, Orthoreovirus
gctattttgcctcttcctagacgttgtcgcaatggaggtgtgtctacctaatggtcatcagatcgtcgactggattaacaatgcatttgaaggacgggtgtcgatttatagtgcacagcaaggatgggataagacaatctcagctcagcctgatatgatggtgtgtggtagcgctgttgtttgcatgcattgcttgggtgtggttggatcattacagcgaaagttgaaccatctgcctcatcataaatgtaatcagcaattgcgtgagcaggattatgttgacctacagtttgctgatcgtgtaaccgctcactggaaacgtggcatgttatcatttgtatctcagatgcatgctatcatgaacgatgtgacacctgaggagcttgaaagagtgagaactgatggtggcatcttggctgagctcaactggcttcaaatagagtctggatcaatgtttcgttcgattcactcaaactggactgacccccttcaggtggtcgaagacctagatactcagctagatcgctattggacagcattgaatttgatgattgattcatcggatctggtgccaaacttcatgatgcgtgacccatcgcatgcctttaatggagtgaagctggagggtgaagcgcgacagactcaattcccgcgcacattcgattccgggtcaaacttgaaatggggtgttatggtatatgattattctgaacttgaaggggattctcagaaaggacgatcttataggagagagatcgttactccagcgaaagactttggtcactttggtttatcccattattctcgcgcaacgacgccaatacttggcaagatgcctgctgtattttctggtatgttaaccgggaactgtaaaatgtatccgtttataaagggcactgctaagctgaaaacggttaagaagctagttgatgctgtgaactacacgtggagttttgagaagatcagatacgctttaggccctggtgggatgacgggatggtataatagaactatgcagcaagcgccaattgtgttgactcctgcggcactgactatgtttccggatatgaccagatttggtgatctacagtatccaatcacgattggcgatccggctgtccttgggtaaacgcctccatcttctcagcgccgggcctgaccaacctggtgtgacgtgggacaggctccattcatc

SEQ ID NO: 46 - protein, 365 aa, Orthoreovirus; identical to SEQ ID NO: 26

SEQ ID NO: 47 - protein, 455 aa, Orthoreovirus
MDPRLREEVVRLIIALTSDNGVSLSKGLESRVSALEKTSQIHSDTILRITQGLDDANKRIIALEQSRDDLVASVSDAQLAISRLESSIGALQTVVNGLDSSVTQLGARVGQLETGLAELRVDHDNLVARVDTAERNIGSLTTELSTLTLRVTSIQADFESRISTLERTAVTSAGAPLSIRNNRMTMGLNDGLTLSGNNLAIRLPGNTGLNIQNGGLQFRFNTDQFQIVNNNLTLKTTVFDSINSRIGATEQSYVASAVTPLRLNSSTKVLDMLIDSSTLEINSSGQLTVRSTSPNLRYPIADVSGGIGMSPNYRFRQSMWIGIVSYSGSGLNWRVQVNSDIFIVDDYIHICLPAFDGFSIADGGDLSLNFVTGLLPPLLTGDTEPAFHNDVVTYGAQTVAIGLSSGGTPQYMSKNLWVEQWQDGVLRLRVEGGGSITHSNSKWPAMTVSYPRSFT

SEQ ID NO: 48 - protein, 709 aa, Orthoreovirus
MGNASSIVQTINVTGDGNVFKPSAETSSTAVPSLSLSPGMLNPGGVPWIAVGDETSVTSPGALRRMTSKDIPETAIINTDNSSGAVPSESALVPYIDEPLVVVTEHAITNFTKAEMALEFNREFLDKMRVLSVSPKYSDLLIYVDCYVGVSARQALNNFQKQVPVITPTRQTMYVDSIQAALKALEKWEIDLRVAQTLLPTNVPIGEVSCPMQSVVKLLDDQLPDDSLIRRYPKEAAVALAKRNGGIQWMDVSEGTVMNEAVNAVAASALAPSASAPPLEEKSKLTEQAMDLVTAAEPEIIASLAPVPAPVFAIPPKPADYNVRTLRIDEATWLRMIPKSMNTPFQIQVTDNTGTNWHLNLRGGTRVVNLDQIAPMRFVLDLGGKSYKETSWDPNGKKVGFIVFQSKIPFELWTAASQIGQATVVNYVQLYAEDSSFTRVMSIIATTSLAYNYEPEQLNKTDPEMNYYLLATFIDSAAITPTNMTQPDVWDALLTMSPLSAGEVTVKGAVVSEVVPADLIGSYTPESLNASLPNDAARCMIDRASKIAEAIKIDDDAGPDEYSPNSVPIQGQLAISQLETGYGVRIFNPKGILSKIASRAMQAFIGDPSTIITQAAPVLSDKNNWIALAQGVKTSLRTKSLSAGVKTAVSKLSSSESIQNWTQGFLDKVSAHFPAPKPDCPTSGDSGESSNRRVKRDSYAGVVKRGYTR

SEQ ID NO: 49 - protein, 736 aa, Orthoreovirus
MAYIAVPAVVDSRSSEAIGLLESFGVDAGADANDVSYQDHDYVLDQLQYMLDGYEAGDVIDALVHKNWLHHSVYCLLPPKSQLLEYWKSNPSAIPDNVDRRLRKRLMLKKDLRKDDEYNQLVRAFKISDVYAPLISSTTSPMTMIQNLNQGEIVYTTTDRVIGARILLYAPRKYYASTLSFTMTKCIIPFGKEVGRVPHSRFNVGTFSSIATPKCFVMSGVDIESIPNEFIKLFYQRVKSVHANILNDISPQIVSDMINRKRLRVHTPSDRRAAQLMHLPYHVKRGASHVDVYKVDVVDMLFEVVDVADGLRNVSRKLTMHTVPVCILEMLGIEIADYCIRQEDGMLTDWFLLLTMLSDGLTDRRTHCQYLINPSSVPPDVILNISITGFINRHTIDVMPDIYDFVKPIGAVLPKGSFKSTIMRVLDSISILGIQIMPRAHVVDSDEVGEQMEPTFEQAVMEIYKGIAGVDSLDDLIKWVLNSDLIPHDDRLGQLFQAFLPLAKDLLAPMARKFYDNSMSEGRLLTFAHADSELLNANYFGHLLRLKIPYITEVNLMIRKNREGGELFQLVLSYLYKMYATSAQPKWFGSLLRLLICPWLHMEKLIGEADPASTSAEIGWHIPREQLMQDGWCGCEDGFIPYVSIRAPRLVIEELMEKNWGQYHAQVIVTDQLVVGEPRRVSAKAVIKGNHLPVKLVSRFACFTLTAKYEMRLSCGHSTGRGAAYSARLAFRSDLA

SEQ ID NO: 50 - DNA, 22 bp, Orthoreovirus
ggattacgcaatgactacagca

SEQ ID NO: 51 - DNA, 21 bp, Orthoreovirus
cctatccacatacttcgccta

SEQ ID NO: 52 - DNA, 23 bp, Orthoreovirus
gcgacactggatcattaacgact

SEQ ID NO: 53 - DNA, 22 bp, Orthoreovirus
ggctcatcccaatactaccact

SEQ ID NO: 54 - DNA, 22 bp, Orthoreovirus
cttgattcgagtgttacccagt

SEQ ID NO: 55 - DNA, 21 bp, Orthoreovirus
taatgatccagtgtcgcgttc

SEQ ID NO: 56 - DNA, 23 bp, Orthoreovirus
cctgcaaatcctgtctcaagctg